(19) United States
(12) Patent Application Publication
(10) Pub. No.: US 2019/0165278 A1
(43) Pub. Date: May 30, 2019

(54) THIOPHENE-CONTAINING TRIARYLAMINE COMPOUNDS

(71) Applicant: Chuanjun Xia, Lawrenceville, NJ (US)

(72) Inventor: Chuanjun Xia, Lawrenceville, NJ (US)

(21) Appl. No.: 16/199,291

(22) Filed: Nov. 26, 2018

Related U.S. Application Data
(60) Provisional application No. 62/591,163, filed on Nov. 27, 2017.

(51) Int. Cl. H01L 51/00; C07D 495/04

(52) U.S. Cl. CPC H01L 51/0061; H01L 51/5056; H01L 51/006; C07D 495/04

(57) ABSTRACT

Thiophene-containing triarylamine compounds are disclosed. The said compounds have a structure of triarylamine, and connect specific thiophene-containing group. The compounds may be used as hole transporting materials, hole injection materials, or the like in an electroluminescent device. Compared to existing hole transporting materials, hole injection materials, the novel compounds can also offer excellent device performance. An electroluminescent device and a formulation comprising the compounds are also disclosed.

This application claims the benefit of U.S. Provisional Application No. 62/591,163, filed Nov. 27, 2017, the entire content of which is incorporated herein by reference.

### 1 FIELD OF THE INVENTION

The present invention relates to compounds for organic electronic devices, such as organic light emitting devices. More specifically, the present invention relates to a thiophene triarylamine structural compound, an organic electroluminescent device and a formulation comprising the compound.

### 2 BACKGROUND ART

An organic electronic device is preferably selected from the group consisting of organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This invention laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since OLED is a self-emitting solid state device, it offers tremendous potential for display and lighting applications. In addition, the inherent properties of organic materials, such as their flexibility, may make them well suited for particular applications such as fabrication on flexible substrates.

OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of a fluorescent OLED is only 25%. This limitation hindered the commercialization of OLED. In 1997, Forrest and Thompson reported phosphorescent OLED, which uses triplet emission from heave metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. Small molecule refers to any organic or organometallic material that is not a polymer. The molecular weight of a small molecule can be large as long as it has well defined structure. Dendrimers with well-defined structures are considered as small molecules. Polymer OLEDs include conjugated polymers and non-conjugated polymers with pendant emitting groups. Small molecule OLED can become a polymer OLED if post polymerization occurred during the fabrication process.

There are various methods for OLED fabrication. Small molecule OLEDs are generally fabricated by vacuum thermal evaporation. Polymer OLEDs are fabricated by solution process, such as spin-coating, ink-jet printing, and nozzle printing. Small molecule OLEDs can also be fabricated by solution process if the materials can be dissolved or dispersed in solvents.

The emitting color of an OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent emitters still suffer from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

In an OLED device, a hole injection layer (HIL) and a hole transporting layer (HTL) are usually provided between the anode and the emissive layer to achieve hole transporting from the anode to the emissive layer. To achieve low device driving voltage and high efficiency, it is critical to have minimum charge injection barrier and appropriate hole-transporting ability from the anode. Various HIL and HTL materials have been developed such as triarylamine compounds with shallow HOMO energy levels, very electron deficient heterocycles, and triarylamine compounds doped with p-type conductivity dopants. In the present invention, a novel thiophene-containing triarylamine compound is disclosed, which can also offer excellent OLED performance for the use as HIL, HTL materials.

### 3 SUMMARY OF THE INVENTION

The present invention aims to provide a series of thiophene-containing triarylamine compounds to solve at least part of above problems. The compound can be used as hole transporting materials, hole injection materials, or the like in an organic electroluminescent device. Compared to existing HIL and HTL materials, these novel compounds can also offer excellent device performance.

According to an embodiment of the present invention, a compound of Formula 1 is disclosed:

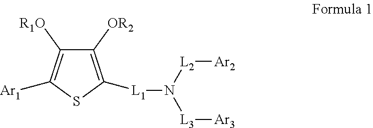

Wherein

R₁ and R₂ are each independently selected from the group consisting of a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, and combinations thereof;

R₁ and R₂ are optionally joined to form a ring;

Ar₁, Ar₂, and Ar₃ are each independently selected from the group consisting of a substituted or unsubstituted aryl group having 6 to 50 ring carbon atoms, a substituted or unsubstituted heteroaryl group having 5 to 50 ring atoms, and combinations thereof;

L₁, L₂, and L₃ are each independently selected from the group consisting of a single bond, an arylene group having 6 to 30 ring carbon atoms, a heteroarylene group having 5 to 30 ring atoms, and combinations thereof.

According to another embodiment, an organic electroluminescent device is disclosed, which comprising:

an anode,

a cathode,

and a series of organic layers, disposed between the anode and the cathode, wherein at least one of the organic layers comprises a compound having Formula 1:

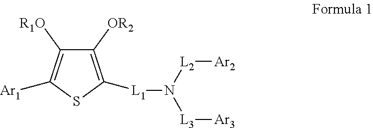

Wherein

R₁ and R₂ are each independently selected from the group consisting of a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, and combinations thereof;

R₁ and R₂ are optionally joined to form a ring;

Ar₁, Ar₂, and Ar₃ are each independently selected from the group consisting of a substituted or unsubstituted aryl group having 6 to 50 ring carbon atoms, a substituted or unsubstituted heteroaryl group having 5 to 50 ring atoms, and combinations thereof;

L₁, L₂, and L₃ are each independently selected from the group consisting of a single bond, an arylene group having 6 to 30 ring carbon atoms, a heteroarylene group having 5 to 30 ring atoms, and combinations thereof.

According to yet another embodiment, a formulation comprising the compound having Formula 1 is also disclosed.

The novel thiophene-containing triarylamine compounds disclosed in the present invention can be used as hole transporting materials, hole injection materials, or the like in an organic electroluminescent device. Compared to existing HIL and HTL materials, these novel compounds can also offer excellent device performance.

### 4 BRIEF DESCRIPTION OF THE DRAWINGS

FIG. 1 schematically shows an organic light emitting device that can incorporate the compound or formulation disclosed herein.

FIG. 2 schematically shows another organic light emitting device that can incorporate the compound or formulation disclosed herein.

FIG. 3 shows the structural Formula 1 of compound disclosed herein.

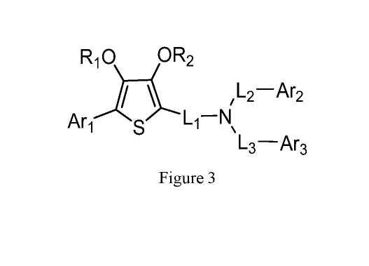

### 5 DETAILED DESCRIPTION

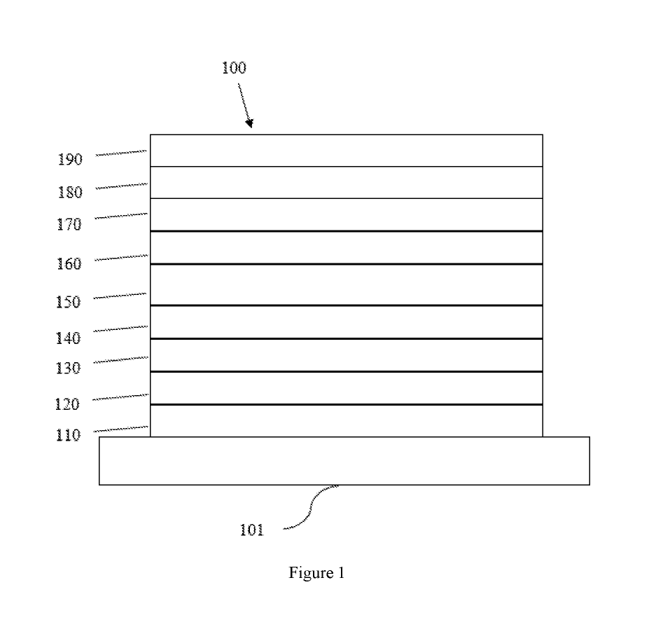

OLEDs can be fabricated on various types of substrates such as glass, plastic, and metal foil. FIG. 1 schematically shows the organic light emitting device 100 without limitation. The figures are not necessarily drawn to scale. Some of the layer in the figure can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, which are incorporated by reference in its entirety.

More examples for each of these layers are available. For example, a flexible and transparent substrate-anode combination is disclosed in U.S. Pat. No. 5,844,363, which is incorporated by reference in its entirety. An example of a p-doped hole transport layer is m-MTDATA doped with F4-TCNQ at a molar ratio of 50:1, as disclosed in U.S. Patent Application Publication No. 2003/0230980, which is incorporated by reference in its entirety. Examples of host materials are disclosed in U.S. Pat. No. 6,303,238 to Thompson et al., which is incorporated by reference in its entirety. An example of an n-doped electron transport layer is BPhen doped with Li at a molar ratio of 1:1, as disclosed in U.S. Patent Application Publication No. 2003/0230980, which is incorporated by reference in its entirety. U.S. Pat. Nos. 5,703,436 and 5,707,745, which are incorporated by reference in their entireties, disclose examples of cathodes including compound cathodes having a thin layer of metal such as Mg:Ag with an overlying transparent, electrically-conductive, sputter-deposited ITO layer. The theory and use of blocking layers is described in more detail in U.S. Pat. No. 6,097,147 and U.S. Patent Application Publication No. 2003/0230980, which are incorporated by reference in their entireties. Examples of injection layers are provided in U.S. Patent Application Publication No. 2004/0174116, which is incorporated by reference in its entirety. A description of protective layers may be found in U.S. Patent Application Publication No. 2004/0174116, which is incorporated by reference in its entirety.

The layered structure described above is provided by way of non-limiting example. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have a two layers of different emitting materials to achieve desired emission spectrum. For another example, the hole transporting layer may comprise a first hole transporting layer and a second hole transporting layer.

In one embodiment, an OLED may be described as having an “organic layer” disposed between a cathode and an anode. This organic layer may comprise a single layer or multiple layers.

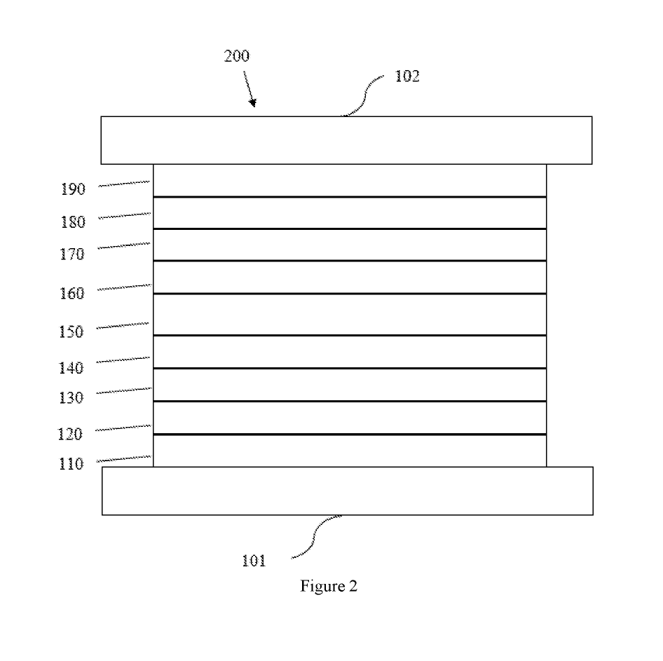

An OLED can be encapsulated by a barrier layer to protect it from harmful species from the environment such as moisture and oxygen. FIG. 2 schematically shows the organic light emitting device 200 without limitation. FIG. 2 differs from FIG. 1 in that the organic light emitting device 200 include a barrier layer 102, which is above the cathode 190. Any material that can provide the barrier function can be used as the barrier layer such as glass and organic-inorganic hybrid layers. The barrier layer should be placed directly or indirectly outside of the OLED device. Multilayer thin film encapsulation was described in U.S. Pat. No. 7,968,146, which is herein incorporated by reference in its entirety.

Devices fabricated in accordance with embodiments of the invention can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (AES-T). Organic, non-metal containing, donor-acceptor luminescent materials may be able to achieve this. The emission in these materials is often characterized as a donor-acceptor charge-transfer (CT) type emission. The spatial separation of the HOMO and LUMO in these donor-acceptor type compounds often results in small ΔES-T. These states may involve CT states. Often, donor-acceptor luminescent materials are constructed by connecting an electron donor moiety such as amino- or carbazole-derivatives and an electron acceptor moiety such as N-containing six-membered aromatic rings.

### Definition of Terms of Substituents

halogen or halide—as used herein includes fluorine, chlorine, bromine, and iodine.

Alkyl—contemplates both straight and branched chain alkyl groups. Examples of the alkyl group include methyl group, ethyl group, propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, 1-methylpentyl group, 2-methylpentyl group, 1-pentylhexyl group, 1-butylpentyl group, 1-heptyloctyl group, 3-methylpentyl group. Additionally, the alkyl group may be optionally substituted. The carbons in the alkyl chain can be replaced by other hetero atoms. Of the above, preferred are methyl group, ethyl group, propyl group, isopropyl group, n-butyl group, s-butyl group, isobutyl group, t-butyl group, n-pentyl group, and neopentyl group.

Cycloalkyl—as used herein contemplates cyclic alkyl groups. Preferred cycloalkyl groups are those containing 4 to 10 ring carbon atoms and includes cyclobutyl, cyclopentyl, cyclohexyl, 4-methylcyclohexyl, 4,4-dimethylcylcohexyl, 1-adamantyl, 2-adamantyl, 1-norbornyl, 2-norbornyl and the like. Additionally, the cycloalkyl group may be optionally substituted. The carbons in the ring can be replaced by other hetero atoms.

Alkenyl—as used herein contemplates both straight and branched chain alkene groups. Preferred alkenyl groups are those containing two to fifteen carbon atoms. Examples of the alkenyl group include vinyl group, allyl group, 1-butenyl group, 2-butenyl group, 3-butenyl group, 1,3-butandienyl group, 1-methylvinyl group, styryl group, 2,2-diphenylvinyl group, 1,2-diphenylvinyl group, 1-methylallyl group, 1,1-dimethylallyl group, 2-methylallyl group, 1-phenylallyl group, 2-phenylallyl group, 3-phenylallyl group, 3,3-diphenylallyl group, 1,2-dimethylallyl group, 1-phenyl1-butenyl group, and 3-phenyl-1-butenyl group. Additionally, the alkenyl group may be optionally substituted.

Alkynyl—as used herein contemplates both straight and branched chain alkyne groups. Preferred alkynyl groups are those containing two to fifteen carbon atoms. Additionally, the alkynyl group may be optionally substituted.

Aryl or aromatic group—as used herein contemplates noncondensed and condensed systems. Preferred aryl groups are those containing six to sixty carbon atoms, preferably six to twenty carbon atoms, more preferably six to twelve carbon atoms. Examples of the aryl group include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Additionally, the aryl group may be optionally substituted. Examples of the non-condensed aryl group include phenyl group, biphenyl-2-yl group, biphenyl-3-yl group, biphenyl-4-yl group, p-terphenyl-4-yl group, p-terphenyl-3-yl group, p-terphenyl-2-yl group, m-terphenyl-4-yl group, m-terphenyl-3-yl group, m-terphenyl-2-yl group, o-tolyl group, m-tolyl group, p-tolyl group, p-t-butylphenyl group, p-(2-phenylpropyl)phenyl group, 4′-methylbiphenylyl group, 4″-t-butyl p-terphenyl-4-yl group, o-cumenyl group, m-cumenyl group, p-cumenyl group, 2,3-xylyl group, 3,4-xylyl group, 2,5-xylyl group, mesityl group, and m-quarterphenyl group.

Heterocyclic group or heterocycle—as used herein contemplates aromatic and non-aromatic cyclic groups. Hetero-aromatic also means heteroaryl. Preferred non-aromatic heterocyclic groups are those containing 3 to 7 ring atoms which includes at least one hetero atom such as nitrogen, oxygen, and sulfur. The heterocyclic group can also be an aromatic heterocyclic group having at least one heteroatom selected from nitrogen atom, oxygen atom, sulfur atom, and selenium atom.

Heteroaryl—as used herein contemplates noncondensed and condensed hetero-aromatic groups that may include from one to five heteroatoms. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—it is represented by —O-Alkyl. Examples and preferred examples thereof are the same as those described above. Examples of the alkoxy group having 1 to 20 carbon atoms, preferably 1 to 6 carbon atoms include methoxy group, ethoxy group, propoxy group, butoxy group, pentyloxy group, and hexyloxy group. The alkoxy group having 3 or more carbon atoms may be linear, cyclic or branched.

Aryloxy—it is represented by —O-Aryl or —O-heteroaryl. Examples and preferred examples thereof are the same as those described above. Examples of the aryloxy group having 6 to 40 carbon atoms include phenoxy group and biphenyloxy group.

Arylalkyl—as used herein contemplates an alkyl group that has an aryl substituent. Additionally, the arylalkyl group may be optionally substituted. Examples of the arylalkyl group include benzyl group, 1-phenylethyl group, 2-phenylethyl group, 1-phenylisopropyl group, 2-phenylisopropyl group, phenyl-t-butyl group, alpha.-naphthylmethyl group, 1-alpha.-naphthylethyl group, 2-alpha-naphthylethyl group, 1-alpha-naphthylisopropyl group, 2-alpha-naphthylisopropyl group, beta-naphthylmethyl group, 1-beta-naphthylethyl group, 2-beta-naphthylethyl group, 1-beta-naphthylisopropyl group, 2-beta-naphthylisopropyl group, p-methylbenzyl group, m-methylbenzyl group, o-methylbenzyl group, p-chlorobenzyl group, m-chlorobenzyl group, o-chlorobenzyl group, p-bromobenzyl group, m-bromobenzyl group, o-bromobenzyl group, p-iodobenzyl group, m-iodobenzyl group, o-iodobenzyl group, p-hydroxybenzyl group, m-hydroxybenzyl group, o-hydroxybenzyl group, p-aminobenzyl group, m-aminobenzyl group, o-aminobenzyl group, p-nitrobenzyl group, m-nitrobenzyl group, o-nitrobenzyl group, p-cyanobenzyl group, m-cyanobenzyl group, o-cyanobenzyl group, 1-hydroxy-2-phenylisopropyl group, and 1-chloro2-phenylisopropyl group. Of the above, preferred are benzyl group, p-cyanobenzyl group, m-cyanobenzyl group, o-cyanobenzyl group, 1-phenylethyl group, 2-phenylethyl group, 1-phenylisopropyl group, and 2-phenylisopropyl group.

The term “aza” in azadibenzofuran, aza-dibenzothiophene, etc. means that one or more of the C—H groups in the respective aromatic fragment are replaced by a nitrogen atom. For example, azatriphenylene encompasses dibenzo[f,h]quinoxaline,dibenzo[f,h]quinoline and other analogues with two or more nitrogens in the ring system. One of ordinary skill in the art can readily envision other nitrogen analogs of the aza-derivatives described above, and all such analogs are intended to be encompassed by the terms as set forth herein.

The alkyl, cycloalkyl, alkenyl, alkynyl, aralkyl, heterocyclic group, aryl, and heteroaryl may be unsubstituted or may be substituted with one or more substituents selected from the group consisting of deuterium, halogen, alkyl, cycloalkyl, arylalkyl, alkoxy, aryloxy, amino, cyclic amino, silyl, alkenyl, cycloalkenyl, heteroalkenyl, alkynyl, aryl, heteroaryl, an acyl group, a carbonyl group, a carboxylic acid group, an ether group, an ester group, a nitrile group, an isonitrile group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In the compounds mentioned in this disclosure, the hydrogen atoms can be partially or fully replaced by deuterium. Other atoms such as carbon and nitrogen, can also be replaced by their other stable isotopes. The replacement by other stable isotopes in the compounds may be preferred due to its enhancements of device efficiency and stability.

In the compounds mentioned in this disclosure, multiple substitutions refer to a range that includes a double substitution, up to the maximum available substitutions.

In the compounds mentioned in this disclosure, the expression that adjacent substituents are optionally joined to form a ring is intended to be taken to mean that two radicals are linked to each other by a chemical bond. This is illustrated by the following scheme:

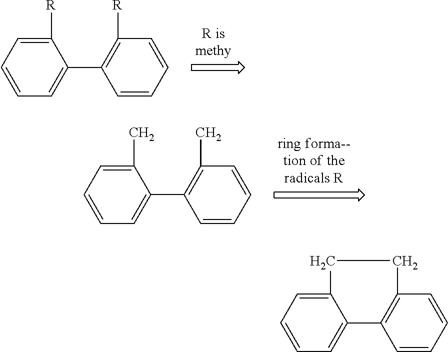

Furthermore, the expression that adjacent substituents are optionally joined to form a ring is also intended to be taken to mean that in the case where one of the two radicals represents hydrogen, the second radical is bonded at a position to which the hydrogen atom was bonded, with formation of a ring. This is illustrated by the following scheme:

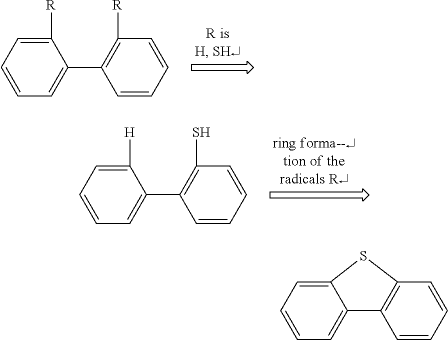

According to an embodiment of the present invention, a compound having Formula 1 is disclosed:

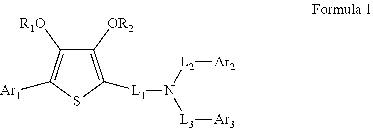

Wherein

R₁ and R₂ are each independently selected from the group consisting of a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, and combinations thereof;

R₁ and R₂ are optionally joined to form a ring;

Each of Ar₁, Ar₂, and Ar₃ are independently selected from the group consisting of a substituted or unsubstituted aryl group having 6 to 50 ring carbon atoms, a substituted or unsubstituted heteroaryl group having 5 to 50 ring atoms, and combinations thereof;

Each of L₁, L₂, and L₃ are independently selected from the group consisting of a single bond, an arylene group having 6 to 30 ring carbon atoms, a heteroarylene group having 5 to 30 ring atoms, and combinations thereof.

In one embodiment, wherein Each of Ar₁, Ar₂, and Ar₃ are independently selected from the group consisting of:

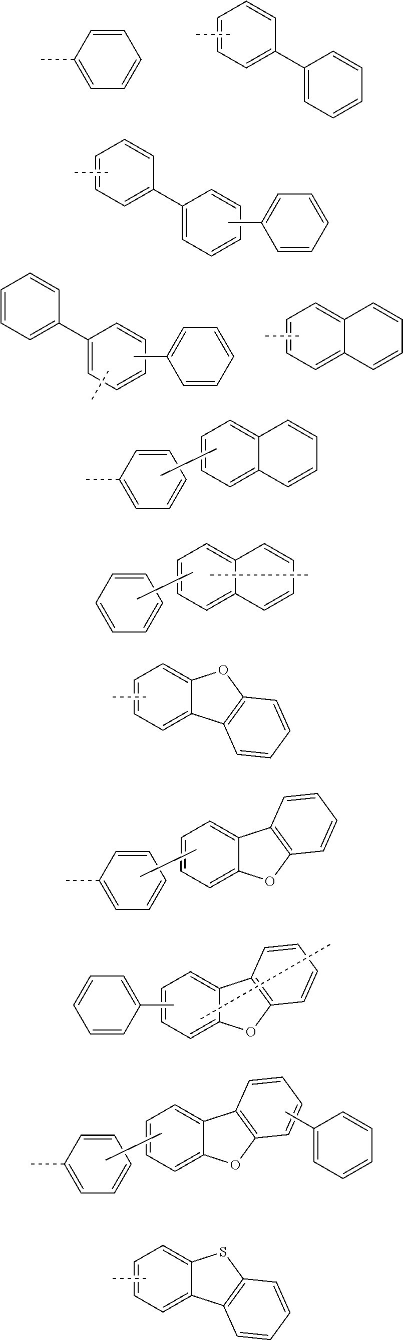

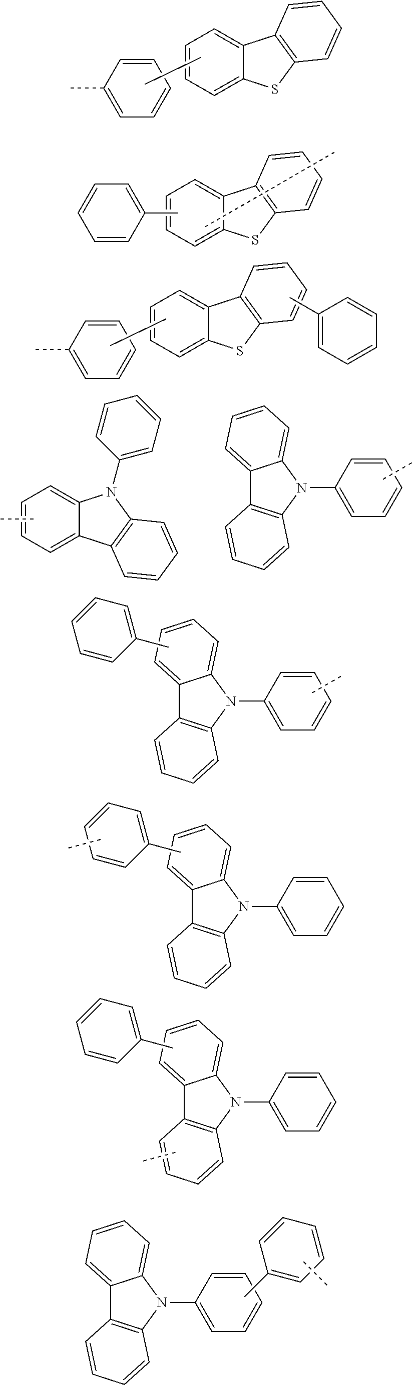

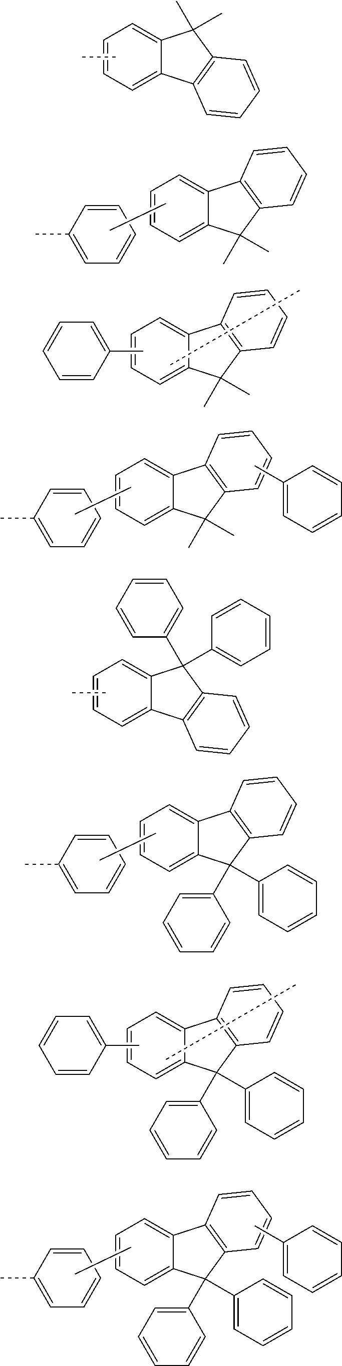

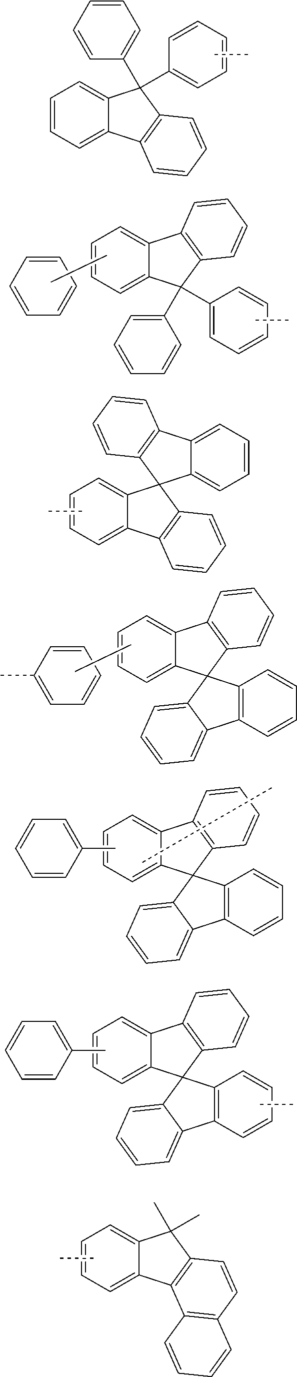

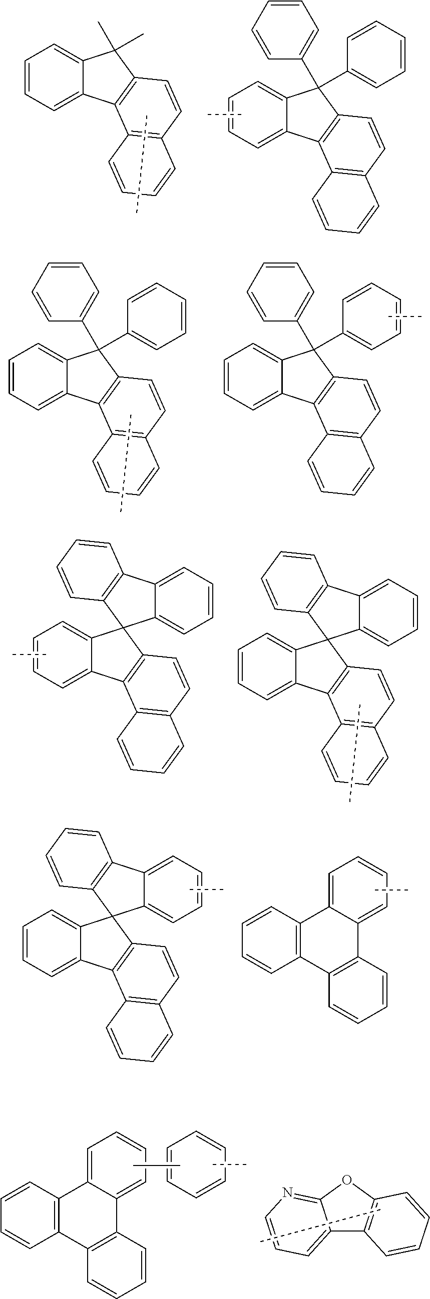

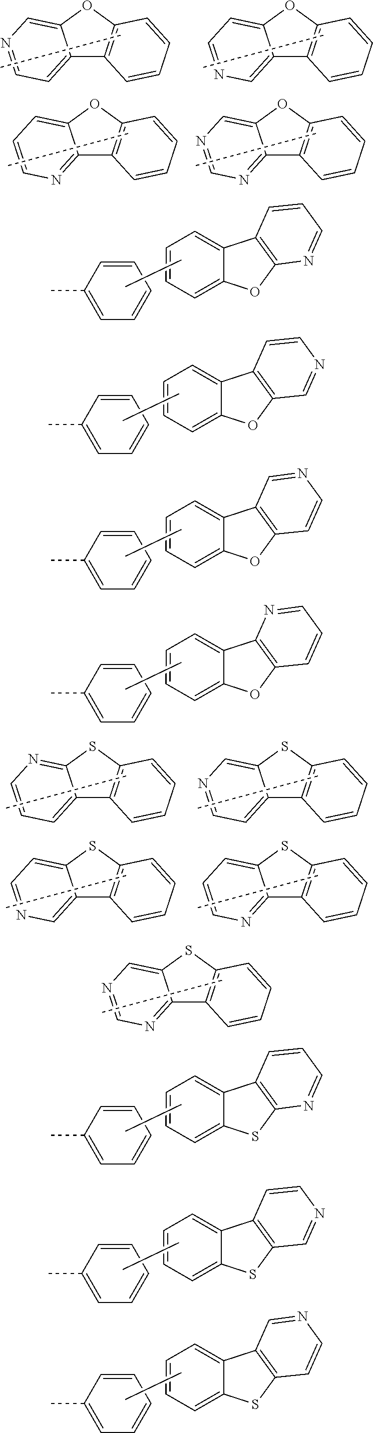

In one embodiment, wherein each of L₁, L₂, and L₃ are independently selected from the group consisting of:

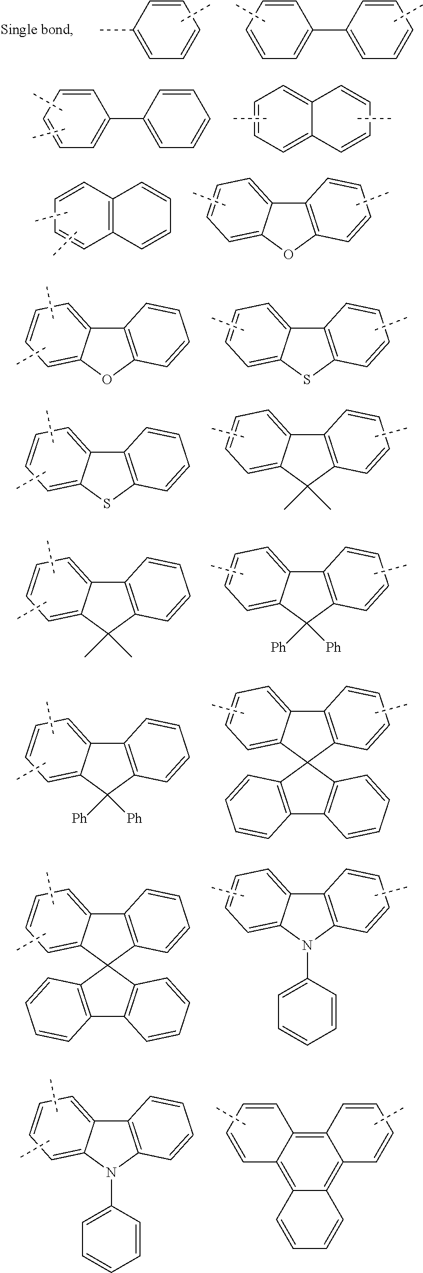

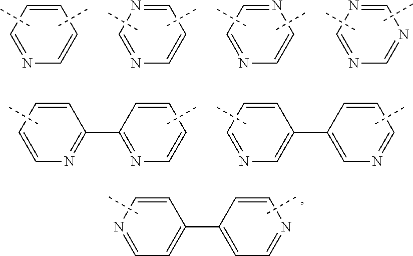

and combinations thereof.

In an embodiment of the present invention, the compound has Formula 2 or Formula 3:

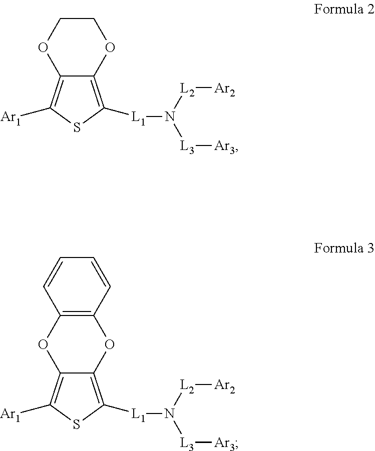

Wherein,

Each of Ar₁, Ar₂, and Ar₃ are independently selected from the group consisting of a substituted or unsubstituted aryl group having 6 to 50 ring carbon atoms, a substituted or unsubstituted heteroaryl group having 5 to 50 ring atoms, and combinations thereof;

Each of L₁, L₂, and L₃ are independently selected from the group consisting of a single bond, an arylene group having 6 to 30 ring carbon atoms, a heteroarylene group having 5 to 30 ring atoms, and combinations thereof.

In an embodiment of the present invention, wherein each of Ar₁ and Ar₂ are independently selected from phenyl, biphenyl, or fluorene; and/or Ar₃ are selected from phenyl, biphenyl, fluorene, dibenzofuran, triphenylene, carbazole, terphenyl, dibenzothiophene, azadibenzofuran, aza-dibenzothiophene, or spirobifluorene.

In an embodiment of the present invention, wherein each of L₁ to L₃ are independently selected from single bond, or phenylene.

In an embodiment, wherein the compound is selected from the group consisting of:

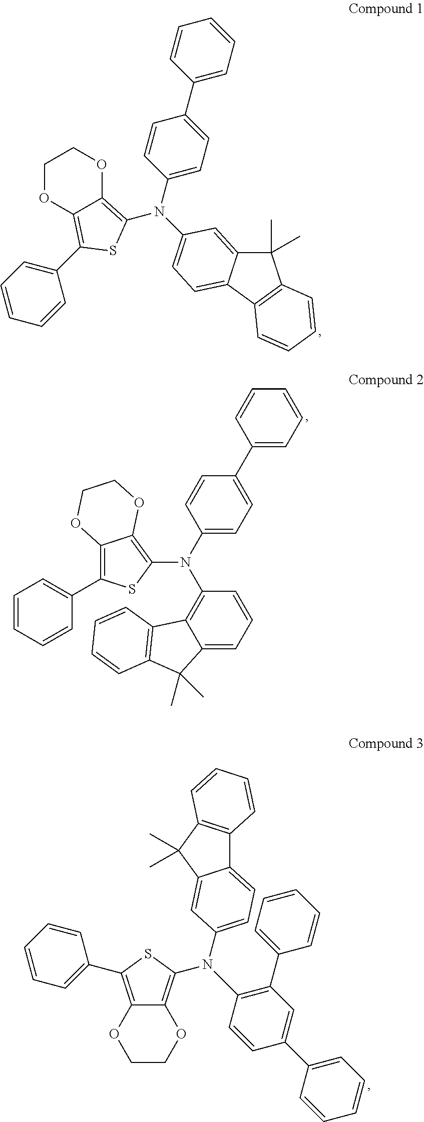

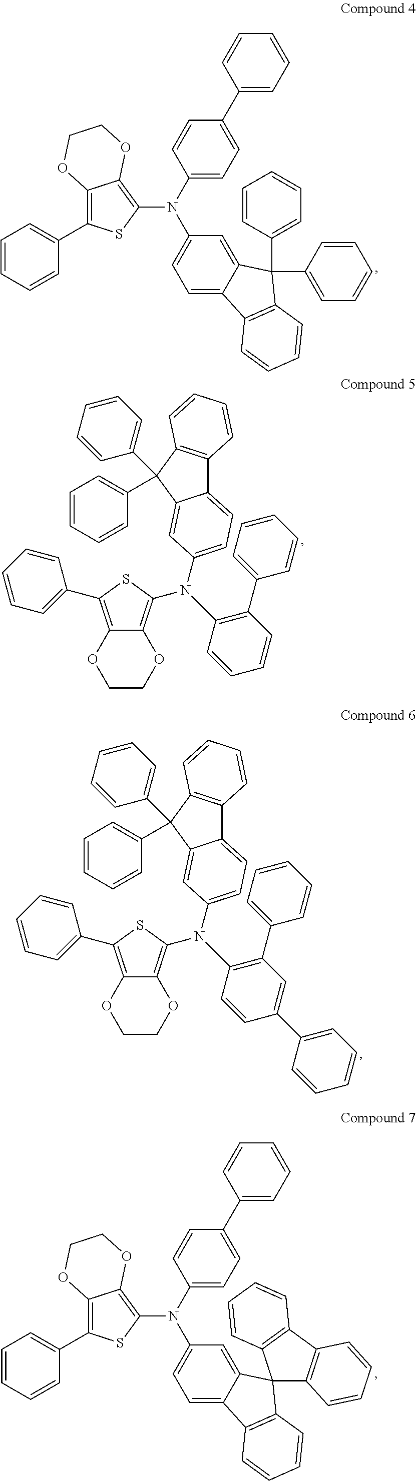 [SMILES: c1ccc(-c2ccc(N(c3ccc4c(c3)C(c3ccccc3)(c3ccccc3)c3ccccc3-4)c3sc(-c4ccccc4)c4c3OCCO4)cc2)cc1 ; c1ccc(-c2ccccc2N(c2ccc3c(c2)C(c2ccccc2)(c2ccccc2)c2ccccc2-3)c2sc(-c3ccccc3)c3c2OCCO3)cc1 ; c1ccc(-c2ccc(N(c3ccc4c(c3)C(c3ccccc3)(c3ccccc3)c3ccccc3-4)c3sc(-c4ccccc4)c4c3OCCO4)c(-c3ccccc3)c2)cc1 ; c1ccc(-c2ccc(N(c3ccc4c(c3)C3(c5ccccc5-c5ccccc53)c3ccccc3-4)c3sc(-c4ccccc4)c4c3OCCO4)cc2)cc1]

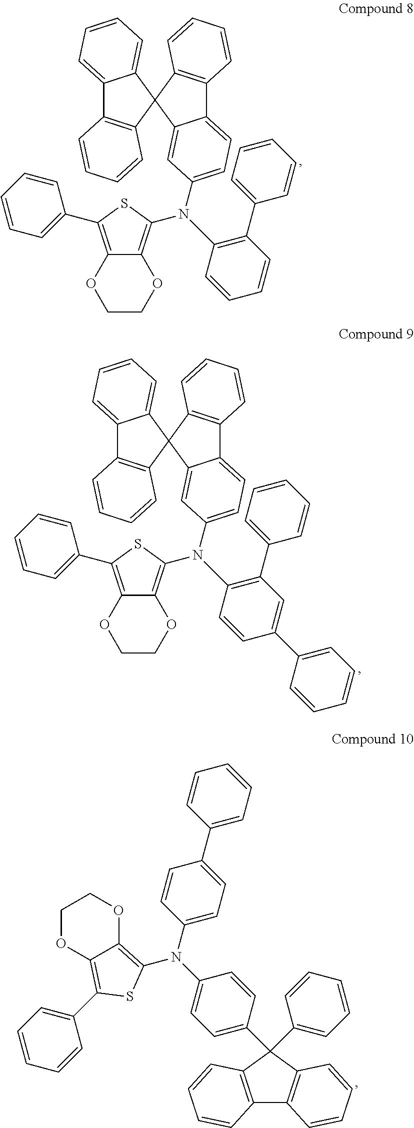 [SMILES: c1ccc(-c2ccccc2N(c2ccc3c(c2)C2(c4ccccc4-c4ccccc42)c2ccccc2-3)c2sc(-c3ccccc3)c3c2OCCO3)cc1 ; c1ccc(-c2ccc(N(c3ccc4c(c3)C3(c5ccccc5-c5ccccc53)c3ccccc3-4)c3sc(-c4ccccc4)c4c3OCCO4)c(-c3ccccc3)c2)cc1 ; c1ccc(-c2ccc(N(c3ccc(C4(c5ccccc5)c5ccccc5-c5ccccc54)cc3)c3sc(-c4ccccc4)c4c3OCCO4)cc2)cc1]

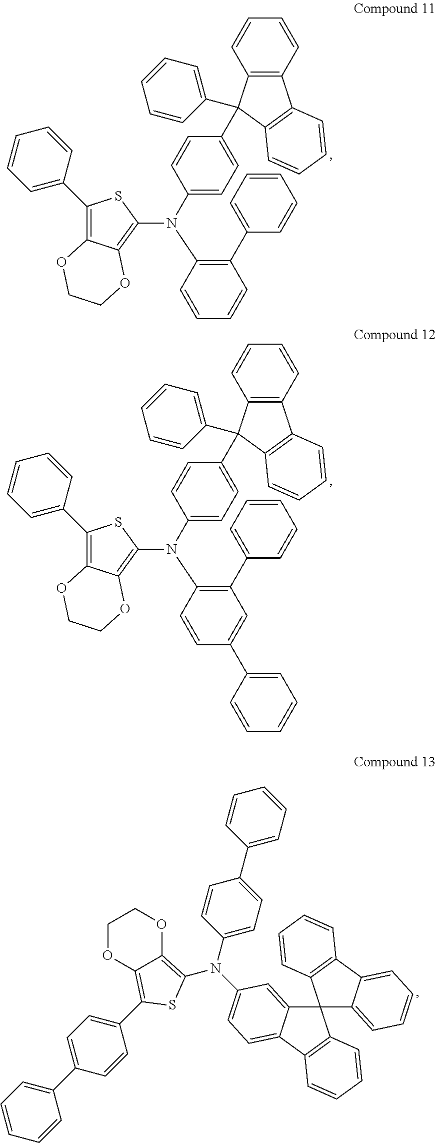

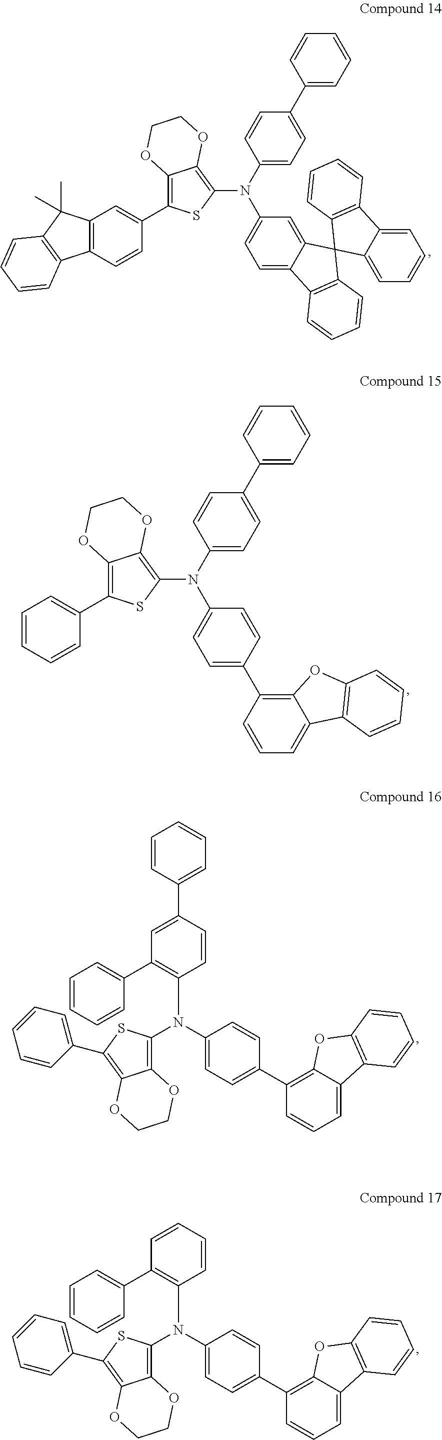 [SMILES: CC1(C)c2ccccc2-c2ccc(-c3sc(N(c4ccc(-c5ccccc5)cc4)c4ccc5c(c4)C4(c6ccccc6-c6ccccc64)c4ccccc4-5)c4c3OCCO4)cc21 ; c1ccc(-c2ccc(N(c3ccc(-c4cccc5c4oc4ccccc45)cc3)c3sc(-c4ccccc4)c4c3OCCO4)cc2)cc1 ; c1ccc(-c2ccc(N(c3ccc(-c4cccc5c4oc4ccccc45)cc3)c3sc(-c4ccccc4)c4c3OCCO4)c(-c3ccccc3)c2)cc1 ; c1ccc(-c2ccccc2N(c2ccc(-c3cccc4c3oc3ccccc34)cc2)c2sc(-c3ccccc3)c3c2OCCO3)cc1]

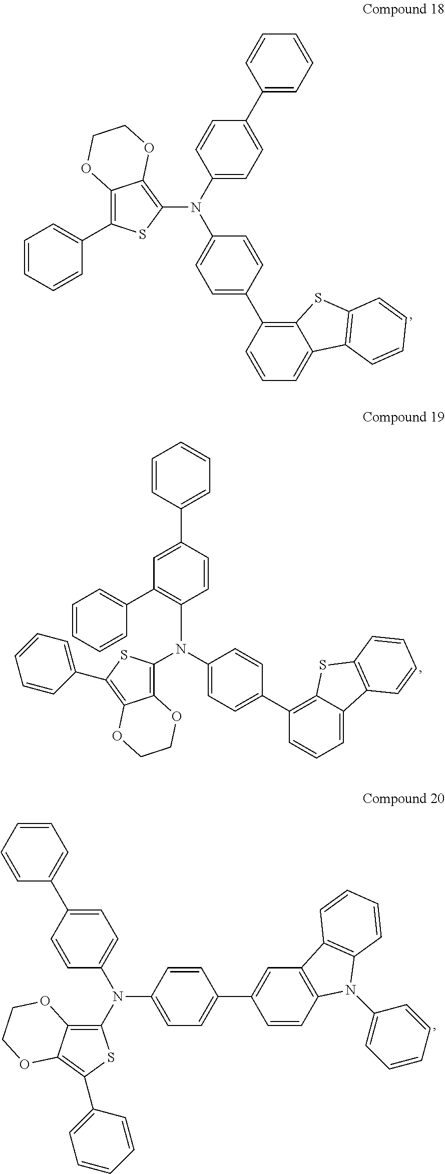 [SMILES: c1ccc(-c2ccc(N(c3ccc(-c4cccc5c4sc4ccccc45)cc3)c3sc(-c4ccccc4)c4c3OCCO4)cc2)cc1 ; c1ccc(-c2ccc(N(c3ccc(-c4cccc5c4sc4ccccc45)cc3)c3sc(-c4ccccc4)c4c3OCCO4)c(-c3ccccc3)c2)cc1 ; c1ccc(-c2ccc(N(c3ccc(-c4ccc5c(c4)c4ccccc4n5-c4ccccc4)cc3)c3sc(-c4ccccc4)c4c3OCCO4)cc2)cc1]

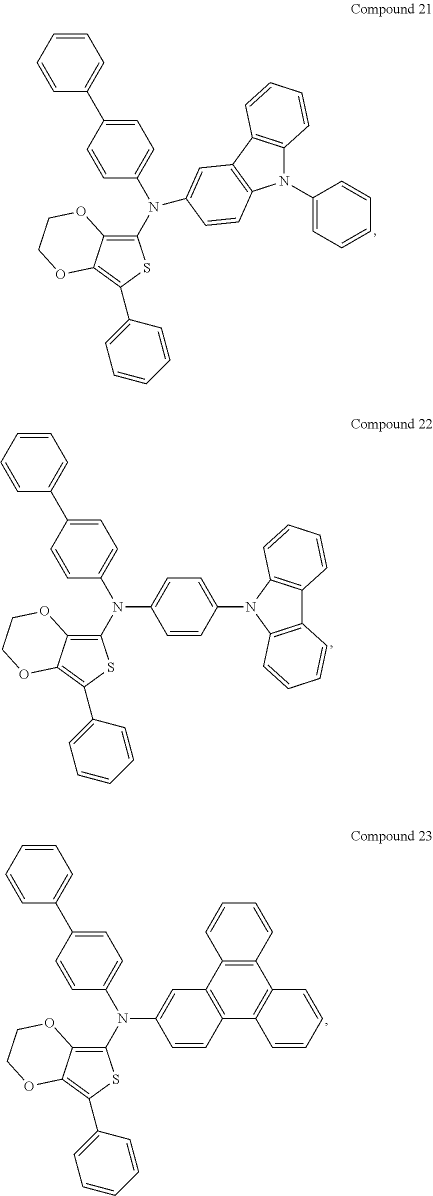 [SMILES: c1ccc(-c2ccc(N(c3ccc4c(c3)c3ccccc3n4-c3ccccc3)c3sc(-c4ccccc4)c4c3OCCO4)cc2)cc1 ; c1ccc(-c2ccc(N(c3ccc(-n4c5ccccc5c5ccccc54)cc3)c3sc(-c4ccccc4)c4c3OCCO4)cc2)cc1 ; c1ccc(-c2ccc(N(c3ccc4c5ccccc5c5ccccc5c4c3)c3sc(-c4ccccc4)c4c3OCCO4)cc2)cc1]

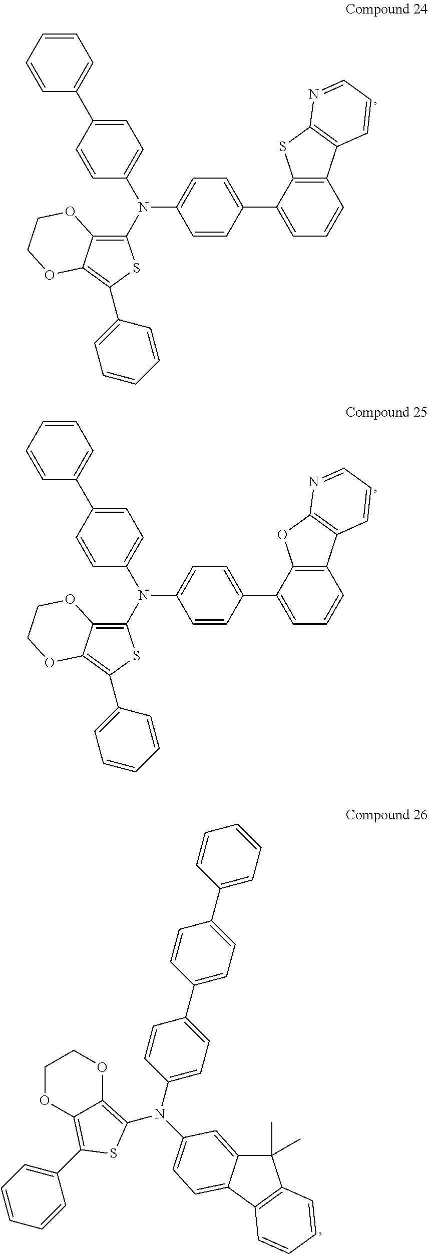 [SMILES: c1ccc(-c2ccc(N(c3ccc(-c4cccc5c4sc4ncccc45)cc3)c3sc(-c4ccccc4)c4c3OCCO4)cc2)cc1 ; c1ccc(-c2ccc(N(c3ccc(-c4cccc5c4oc4ncccc45)cc3)c3sc(-c4ccccc4)c4c3OCCO4)cc2)cc1 ; CC1(C)c2ccccc2-c2ccc(N(c3ccc(-c4ccc(-c5ccccc5)cc4)cc3)c3sc(-c4ccccc4)c4c3OCCO4)cc21]

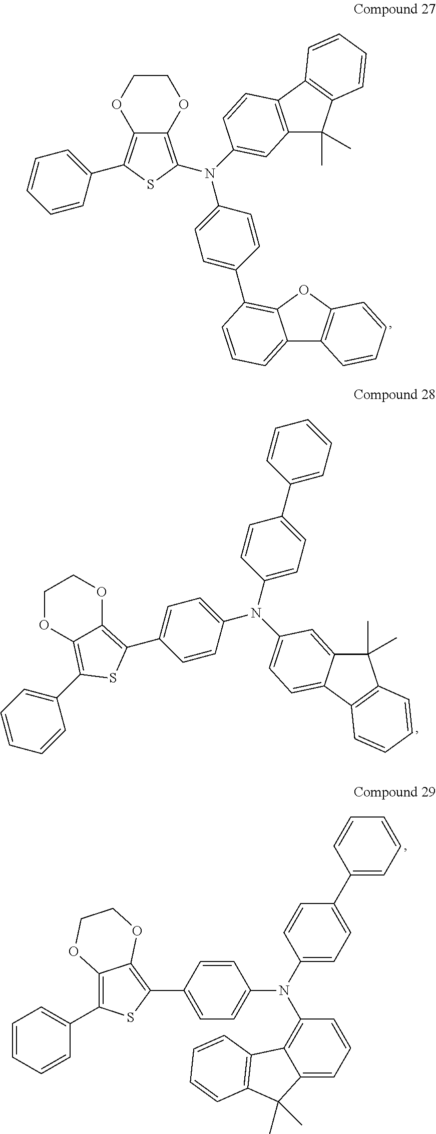 [SMILES: CC1(C)c2ccccc2-c2ccc(N(c3ccc(-c4cccc5c4oc4ccccc45)cc3)c3sc(-c4ccccc4)c4c3OCCO4)cc21 ; CC1(C)c2ccccc2-c2ccc(N(c3ccc(-c4ccccc4)cc3)c3ccc(-c4sc(-c5ccccc5)c5c4OCCO5)cc3)cc21 ; CC1(C)c2ccccc2-c2c(N(c3ccc(-c4ccccc4)cc3)c3ccc(-c4sc(-c5ccccc5)c5c4OCCO5)cc3)cccc21]

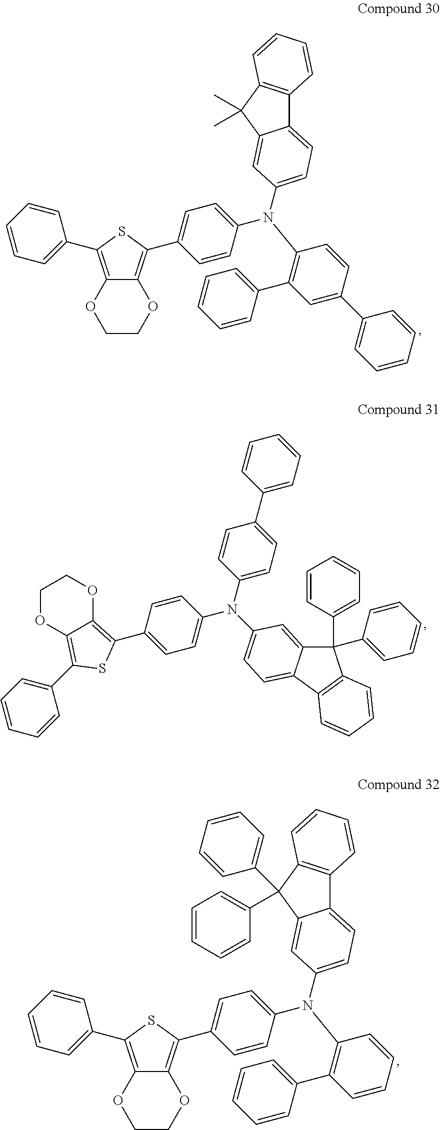 [SMILES: CC1(C)c2ccccc2-c2ccc(N(c3ccc(-c4sc(-c5ccccc5)c5c4OCCO5)cc3)c3ccc(-c4ccccc4)cc3-c3ccccc3)cc21 ; c1ccc(-c2ccc(N(c3ccc(-c4sc(-c5ccccc5)c5c4OCCO5)cc3)c3ccc4c(c3)C(c3ccccc3)(c3ccccc3)c3ccccc3-4)cc2)cc1 ; c1ccc(-c2ccccc2N(c2ccc(-c3sc(-c4ccccc4)c4c3OCCO4)cc2)c2ccc3c(c2)C(c2ccccc2)(c2ccccc2)c2ccccc2-3)cc1]

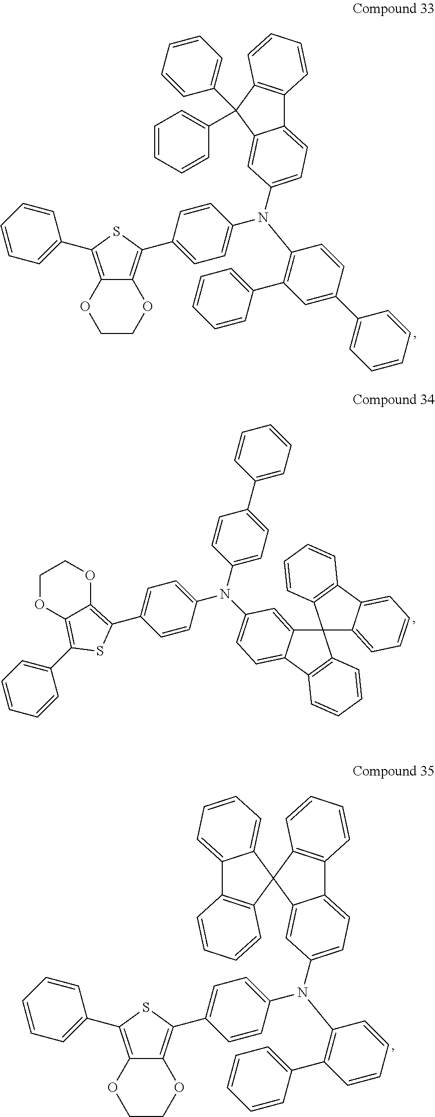 [SMILES: c1ccc(-c2ccc(N(c3ccc(-c4sc(-c5ccccc5)c5c4OCCO5)cc3)c3ccc4c(c3)C(c3ccccc3)(c3ccccc3)c3ccccc3-4)c(-c3ccccc3)c2)cc1 ; c1ccc(-c2ccc(N(c3ccc(-c4sc(-c5ccccc5)c5c4OCCO5)cc3)c3ccc4c(c3)C3(c5ccccc5-c5ccccc53)c3ccccc3-4)cc2)cc1 ; c1ccc(-c2ccccc2N(c2ccc(-c3sc(-c4ccccc4)c4c3OCCO4)cc2)c2ccc3c(c2)C2(c4ccccc4-c4ccccc42)c2ccccc2-3)cc1]

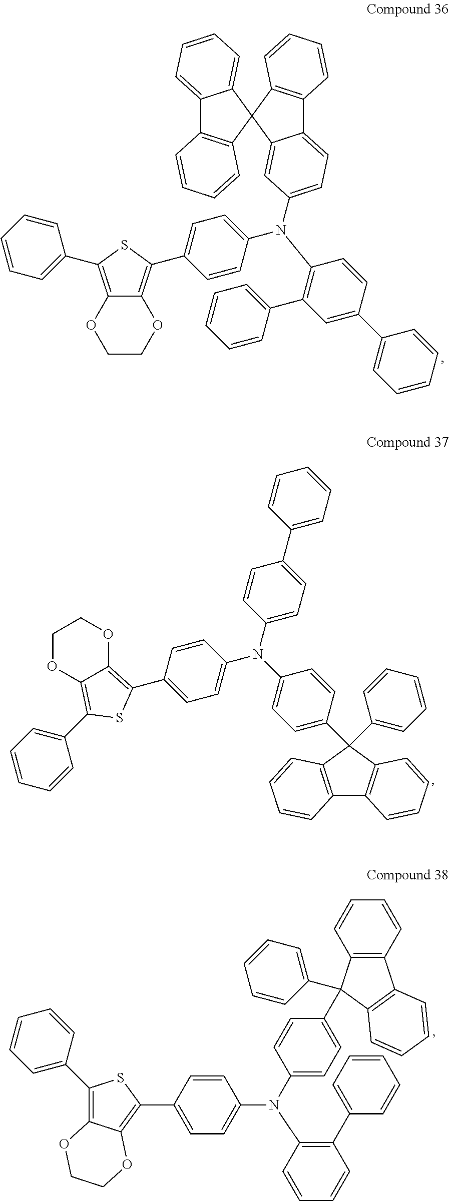 [SMILES: c1ccc(-c2ccc(N(c3ccc(-c4sc(-c5ccccc5)c5c4OCCO5)cc3)c3ccc4c(c3)C3(c5ccccc5-c5ccccc53)c3ccccc3-4)c(-c3ccccc3)c2)cc1 ; c1ccc(-c2ccc(N(c3ccc(-c4sc(-c5ccccc5)c5c4OCCO5)cc3)c3ccc(C4(c5ccccc5)c5ccccc5-c5ccccc54)cc3)cc2)cc1 ; c1ccc(-c2ccccc2N(c2ccc(-c3sc(-c4ccccc4)c4c3OCCO4)cc2)c2ccc(C3(c4ccccc4)c4ccccc4-c4ccccc43)cc2)cc1]

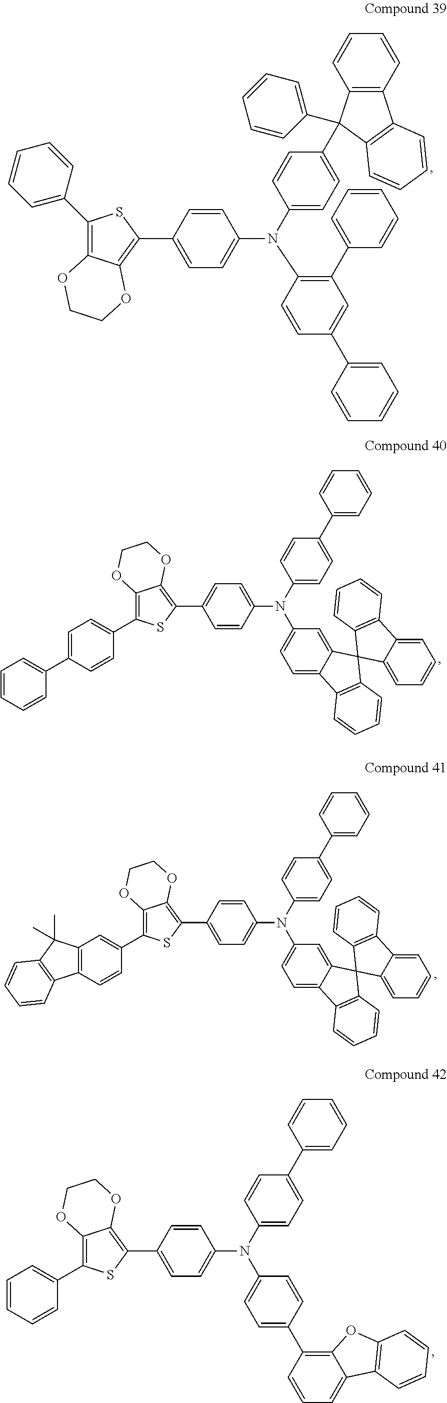 [SMILES: c1ccc(-c2ccc(N(c3ccc(-c4sc(-c5ccccc5)c5c4OCCO5)cc3)c3ccc(C4(c5ccccc5)c5ccccc5-c5ccccc54)cc3)c(-c3ccccc3)c2)cc1 ; c1ccc(-c2ccc(-c3sc(-c4ccc(N(c5ccc(-c6ccccc6)cc5)c5ccc6c(c5)C5(c7ccccc7-c7ccccc75)c5ccccc5-6)cc4)c4c3OCCO4)cc2)cc1 ; CC1(C)c2ccccc2-c2ccc(-c3sc(-c4ccc(N(c5ccc(-c6ccccc6)cc5)c5ccc6c(c5)C5(c7ccccc7-c7ccccc75)c5ccccc5-6)cc4)c4c3OCCO4)cc21 ; c1ccc(-c2ccc(N(c3ccc(-c4sc(-c5ccccc5)c5c4OCCO5)cc3)c3ccc(-c4cccc5c4oc4ccccc45)cc3)cc2)cc1]

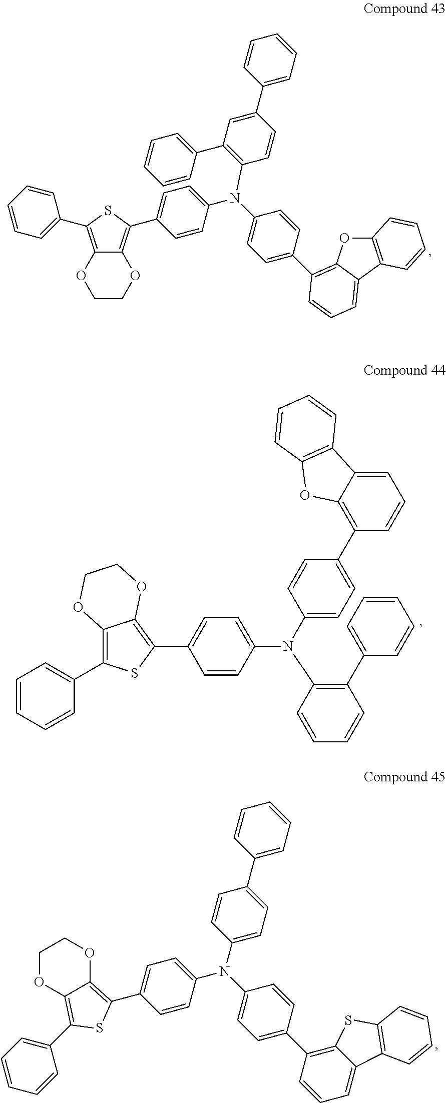 [SMILES: c1ccc(-c2ccc(N(c3ccc(-c4sc(-c5ccccc5)c5c4OCCO5)cc3)c3ccc(-c4cccc5c4oc4ccccc45)cc3)c(-c3ccccc3)c2)cc1 ; c1ccc(-c2ccccc2N(c2ccc(-c3sc(-c4ccccc4)c4c3OCCO4)cc2)c2ccc(-c3cccc4c3oc3ccccc34)cc2)cc1 ; c1ccc(-c2ccc(N(c3ccc(-c4sc(-c5ccccc5)c5c4OCCO5)cc3)c3ccc(-c4cccc5c4sc4ccccc45)cc3)cc2)cc1]

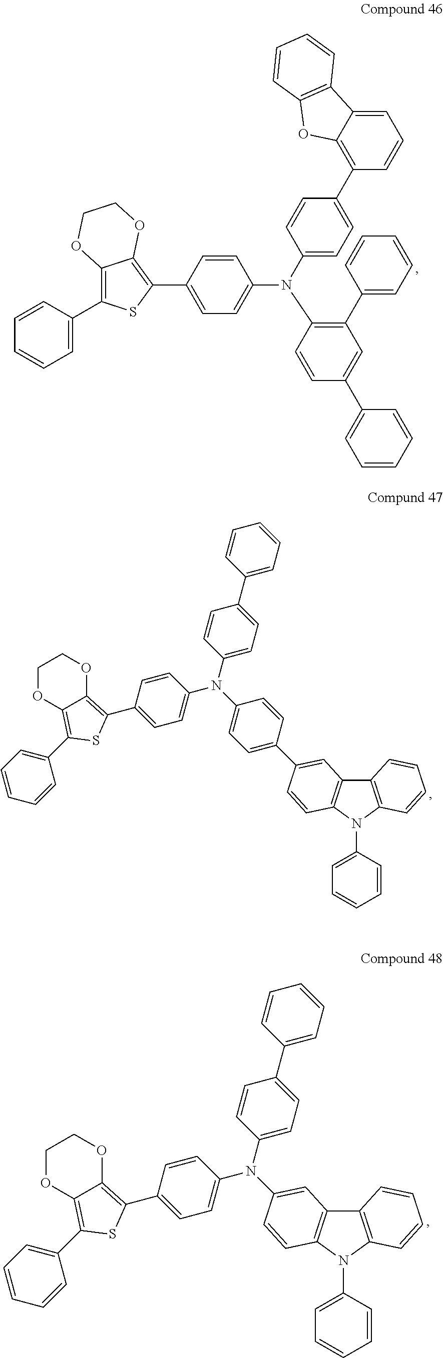 [SMILES: c1ccc(-c2ccc(N(c3ccc(-c4sc(-c5ccccc5)c5c4OCCO5)cc3)c3ccc(-c4cccc5c4oc4ccccc45)cc3)c(-c3ccccc3)c2)cc1 ; c1ccc(-c2ccc(N(c3ccc(-c4ccc5c(c4)c4ccccc4n5-c4ccccc4)cc3)c3ccc(-c4sc(-c5ccccc5)c5c4OCCO5)cc3)cc2)cc1 ; c1ccc(-c2ccc(N(c3ccc(-c4sc(-c5ccccc5)c5c4OCCO5)cc3)c3ccc4c(c3)c3ccccc3n4-c3ccccc3)cc2)cc1]

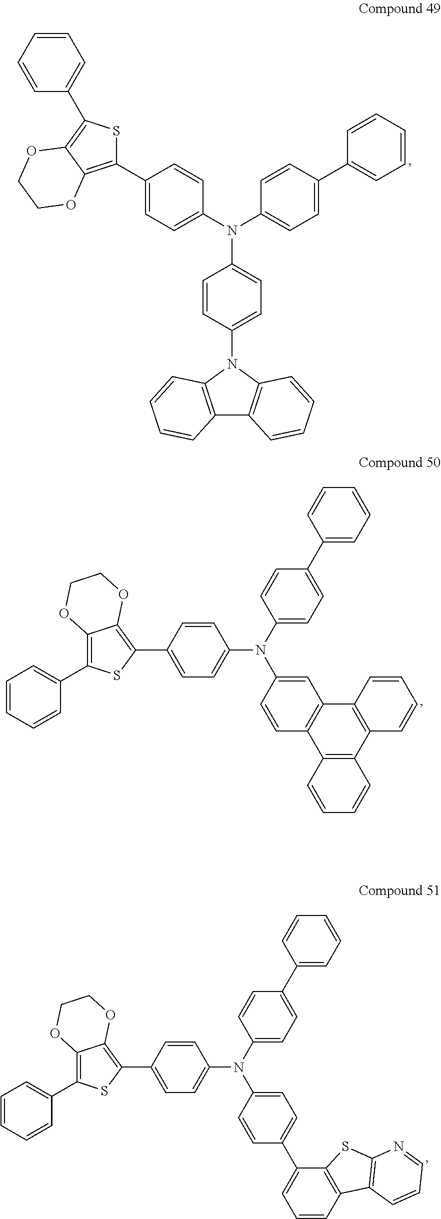 [SMILES: c1ccc(-c2ccc(N(c3ccc(-c4sc(-c5ccccc5)c5c4OCCO5)cc3)c3ccc(-n4c5ccccc5c5ccccc54)cc3)cc2)cc1 ; c1ccc(-c2ccc(N(c3ccc(-c4sc(-c5ccccc5)c5c4OCCO5)cc3)c3ccc4c5ccccc5c5ccccc5c4c3)cc2)cc1 ; c1ccc(-c2ccc(N(c3ccc(-c4sc(-c5ccccc5)c5c4OCCO5)cc3)c3ccc(-c4cccc5c4sc4ncccc45)cc3)cc2)cc1]

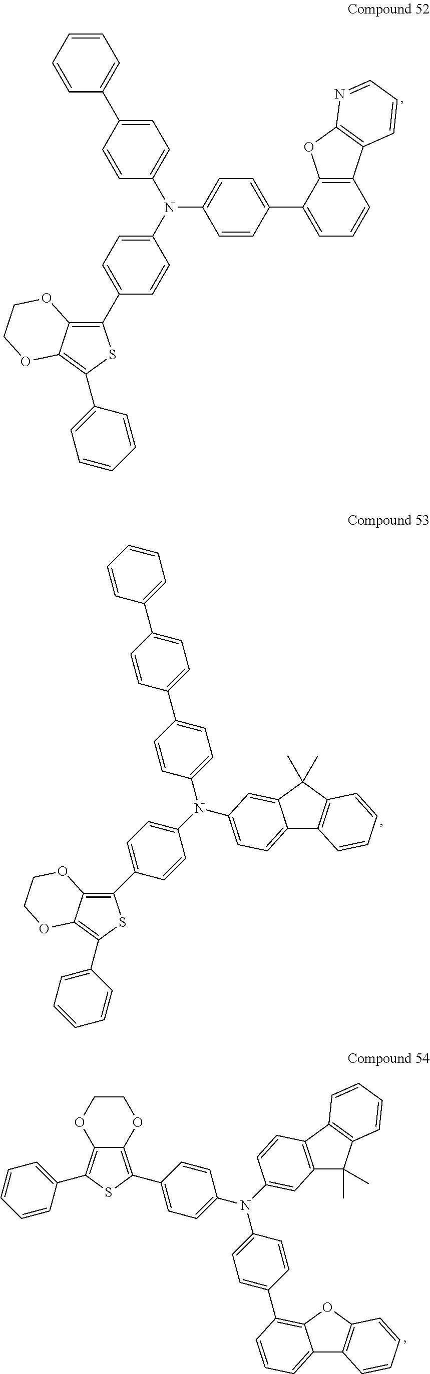 [SMILES: c1ccc(-c2ccc(N(c3ccc(-c4sc(-c5ccccc5)c5c4OCCO5)cc3)c3ccc(-c4cccc5c4oc4ncccc45)cc3)cc2)cc1 ; CC1(C)c2ccccc2-c2ccc(N(c3ccc(-c4ccc(-c5ccccc5)cc4)cc3)c3ccc(-c4sc(-c5ccccc5)c5c4OCCO5)cc3)cc21 ; CC1(C)c2ccccc2-c2ccc(N(c3ccc(-c4sc(-c5ccccc5)c5c4OCCO5)cc3)c3ccc(-c4cccc5c4oc4ccccc45)cc3)cc21]

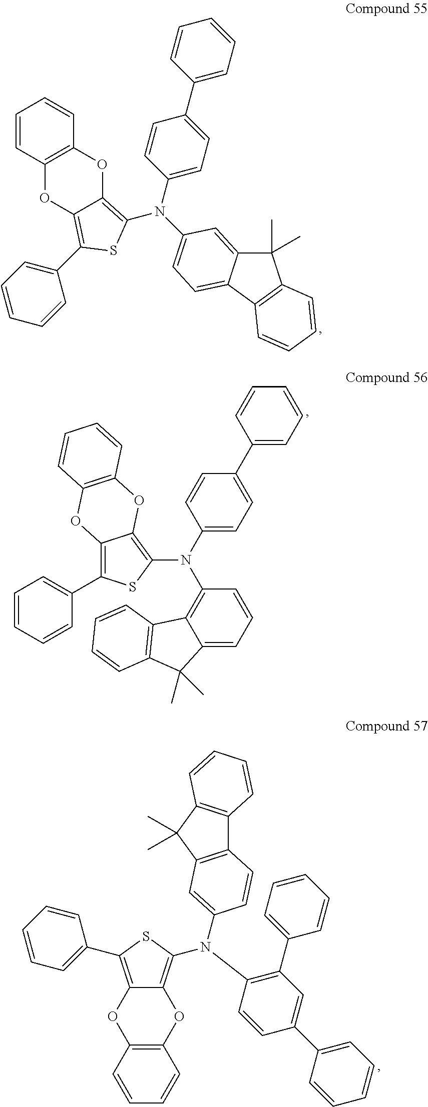

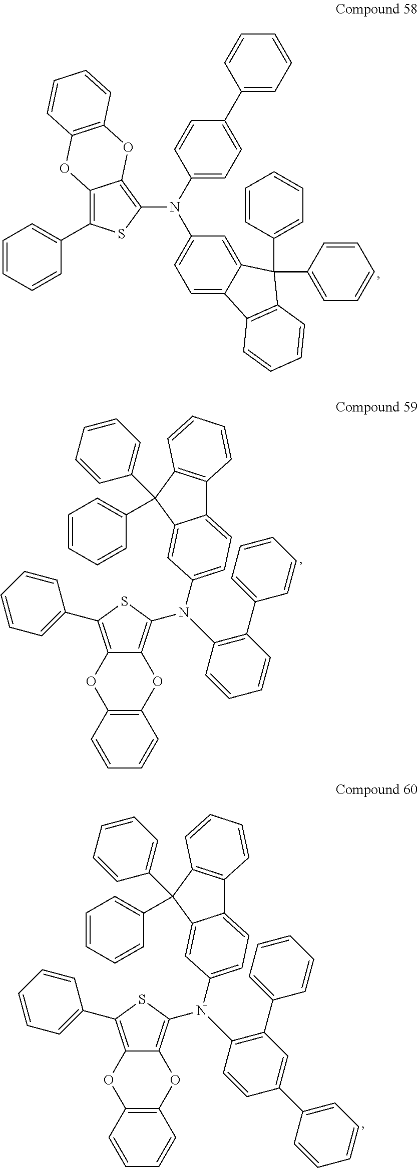 [SMILES: c1ccc(-c2ccc(N(c3ccc4c(c3)C(c3ccccc3)(c3ccccc3)c3ccccc3-4)c3sc(-c4ccccc4)c4c3Oc3ccccc3O4)cc2)cc1 ; c1ccc(-c2ccccc2N(c2ccc3c(c2)C(c2ccccc2)(c2ccccc2)c2ccccc2-3)c2sc(-c3ccccc3)c3c2Oc2ccccc2O3)cc1 ; c1ccc(-c2ccc(N(c3ccc4c(c3)C(c3ccccc3)(c3ccccc3)c3ccccc3-4)c3sc(-c4ccccc4)c4c3Oc3ccccc3O4)c(-c3ccccc3)c2)cc1]

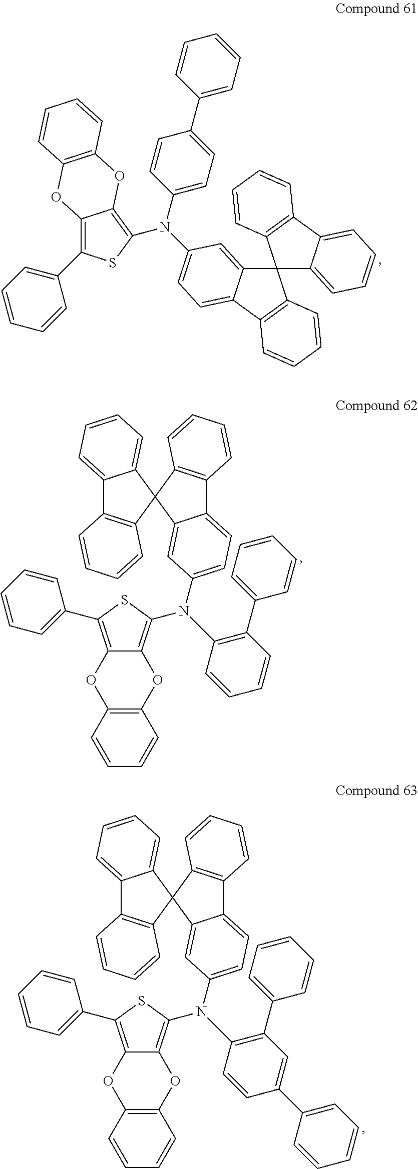 [SMILES: c1ccc(-c2ccc(N(c3ccc4c(c3)C3(c5ccccc5-c5ccccc53)c3ccccc3-4)c3sc(-c4ccccc4)c4c3Oc3ccccc3O4)cc2)cc1 ; c1ccc(-c2ccccc2N(c2ccc3c(c2)C2(c4ccccc4-c4ccccc42)c2ccccc2-3)c2sc(-c3ccccc3)c3c2Oc2ccccc2O3)cc1 ; c1ccc(-c2ccc(N(c3ccc4c(c3)C3(c5ccccc5-c5ccccc53)c3ccccc3-4)c3sc(-c4ccccc4)c4c3Oc3ccccc3O4)c(-c3ccccc3)c2)cc1]

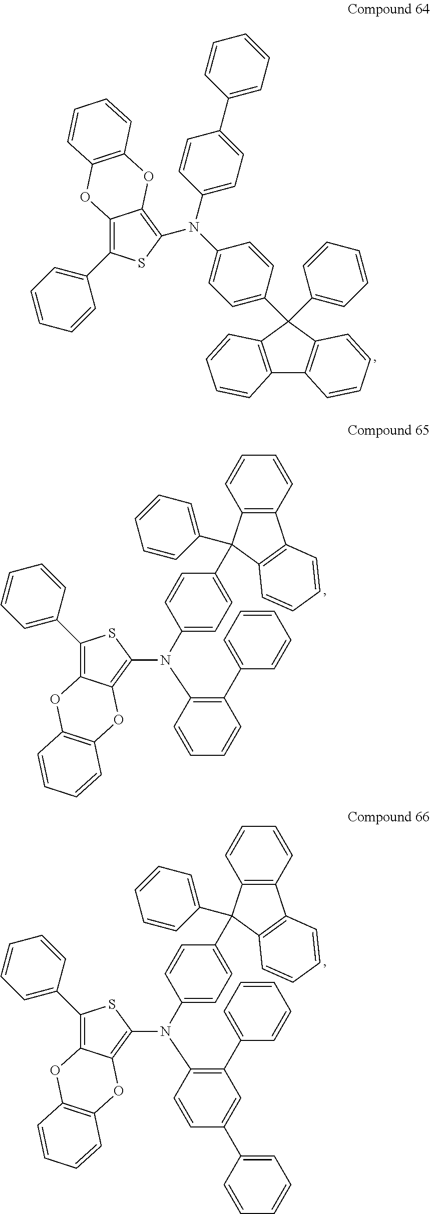 [SMILES: c1ccc(-c2ccc(N(c3ccc(C4(c5ccccc5)c5ccccc5-c5ccccc54)cc3)c3sc(-c4ccccc4)c4c3Oc3ccccc3O4)cc2)cc1 ; c1ccc(-c2ccccc2N(c2ccc(C3(c4ccccc4)c4ccccc4-c4ccccc43)cc2)c2sc(-c3ccccc3)c3c2Oc2ccccc2O3)cc1 ; c1ccc(-c2ccc(N(c3ccc(C4(c5ccccc5)c5ccccc5-c5ccccc54)cc3)c3sc(-c4ccccc4)c4c3Oc3ccccc3O4)c(-c3ccccc3)c2)cc1]

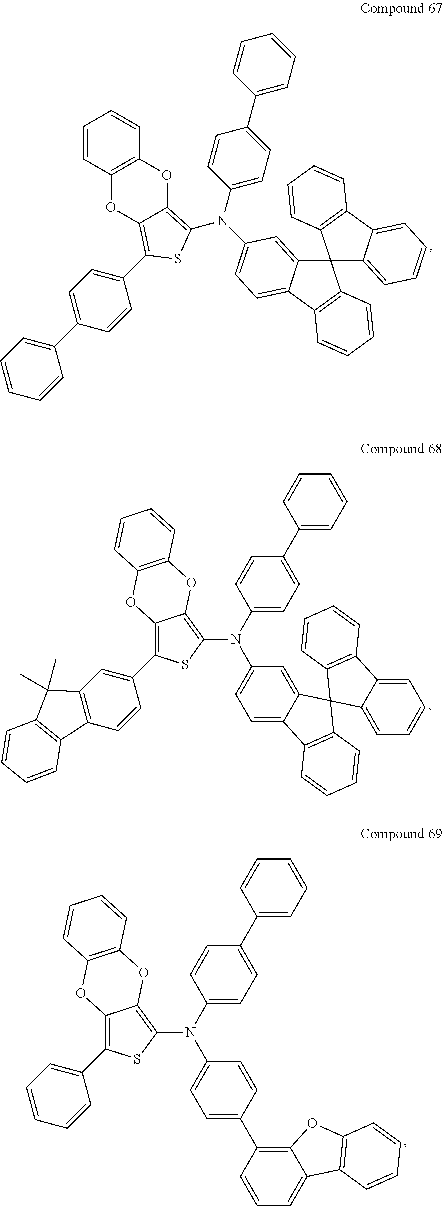 [SMILES: c1ccc(-c2ccc(-c3sc(N(c4ccc(-c5ccccc5)cc4)c4ccc5c(c4)C4(c6ccccc6-c6ccccc64)c4ccccc4-5)c4c3Oc3ccccc3O4)cc2)cc1 ; CC1(C)c2ccccc2-c2ccc(-c3sc(N(c4ccc(-c5ccccc5)cc4)c4ccc5c(c4)C4(c6ccccc6-c6ccccc64)c4ccccc4-5)c4c3Oc3ccccc3O4)cc21 ; c1ccc(-c2ccc(N(c3ccc(-c4cccc5c4oc4ccccc45)cc3)c3sc(-c4ccccc4)c4c3Oc3ccccc3O4)cc2)cc1]

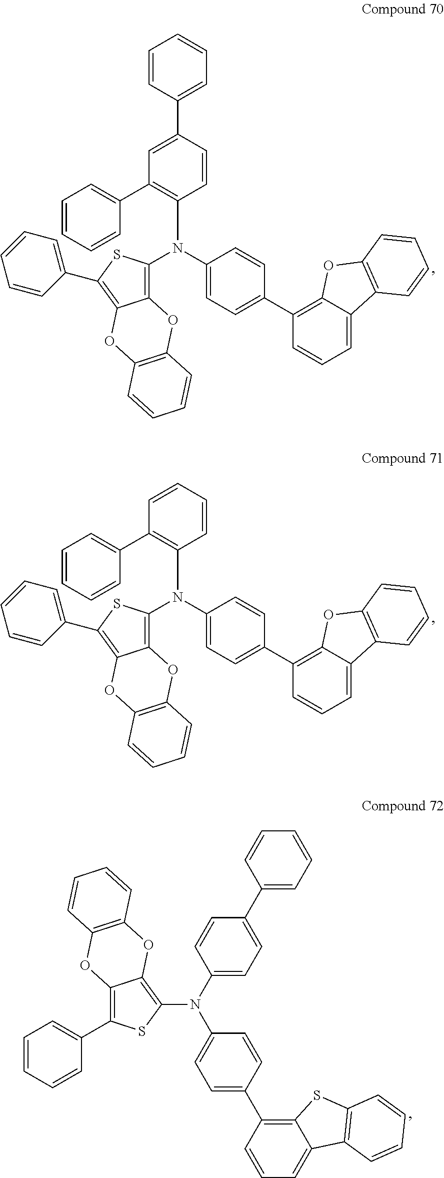 [SMILES: c1ccc(-c2ccc(N(c3ccc(-c4cccc5c4oc4ccccc45)cc3)c3sc(-c4ccccc4)c4c3Oc3ccccc3O4)c(-c3ccccc3)c2)cc1 ; c1ccc(-c2ccccc2N(c2ccc(-c3cccc4c3oc3ccccc34)cc2)c2sc(-c3ccccc3)c3c2Oc2ccccc2O3)cc1 ; c1ccc(-c2ccc(N(c3ccc(-c4cccc5c4sc4ccccc45)cc3)c3sc(-c4ccccc4)c4c3Oc3ccccc3O4)cc2)cc1]

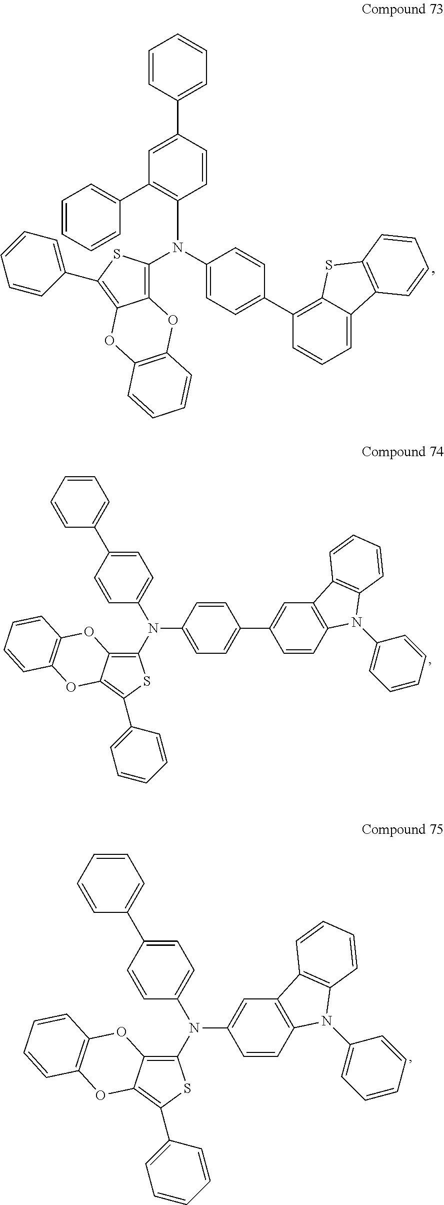 [SMILES: c1ccc(-c2ccc(N(c3ccc(-c4cccc5c4sc4ccccc45)cc3)c3sc(-c4ccccc4)c4c3Oc3ccccc3O4)c(-c3ccccc3)c2)cc1 ; c1ccc(-c2ccc(N(c3ccc(-c4ccc5c(c4)c4ccccc4n5-c4ccccc4)cc3)c3sc(-c4ccccc4)c4c3Oc3ccccc3O4)cc2)cc1 ; c1ccc(-c2ccc(N(c3ccc4c(c3)c3ccccc3n4-c3ccccc3)c3sc(-c4ccccc4)c4c3Oc3ccccc3O4)cc2)cc1]

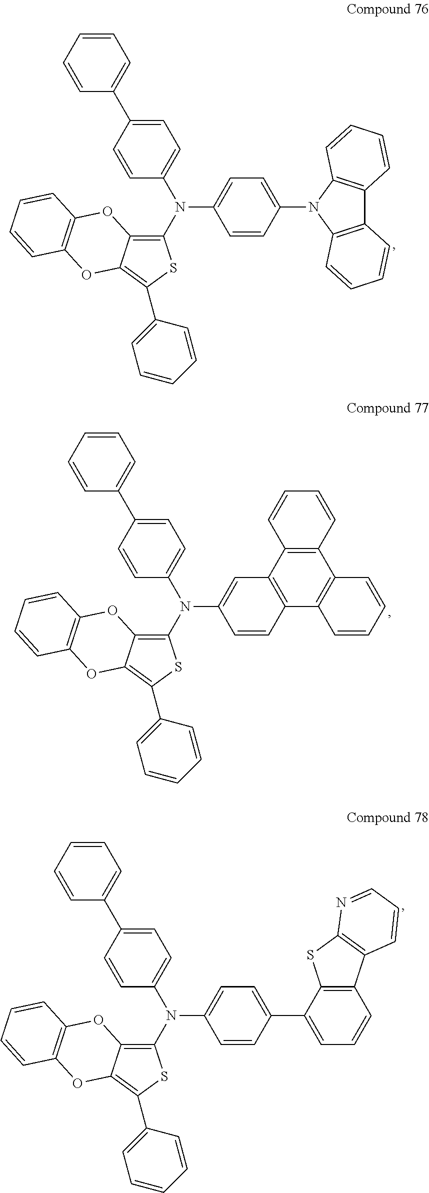 [SMILES: c1ccc(-c2ccc(N(c3ccc(-n4c5ccccc5c5ccccc54)cc3)c3sc(-c4ccccc4)c4c3Oc3ccccc3O4)cc2)cc1 ; c1ccc(-c2ccc(N(c3ccc4c5ccccc5c5ccccc5c4c3)c3sc(-c4ccccc4)c4c3Oc3ccccc3O4)cc2)cc1 ; c1ccc(-c2ccc(N(c3ccc(-c4cccc5c4sc4ncccc45)cc3)c3sc(-c4ccccc4)c4c3Oc3ccccc3O4)cc2)cc1]

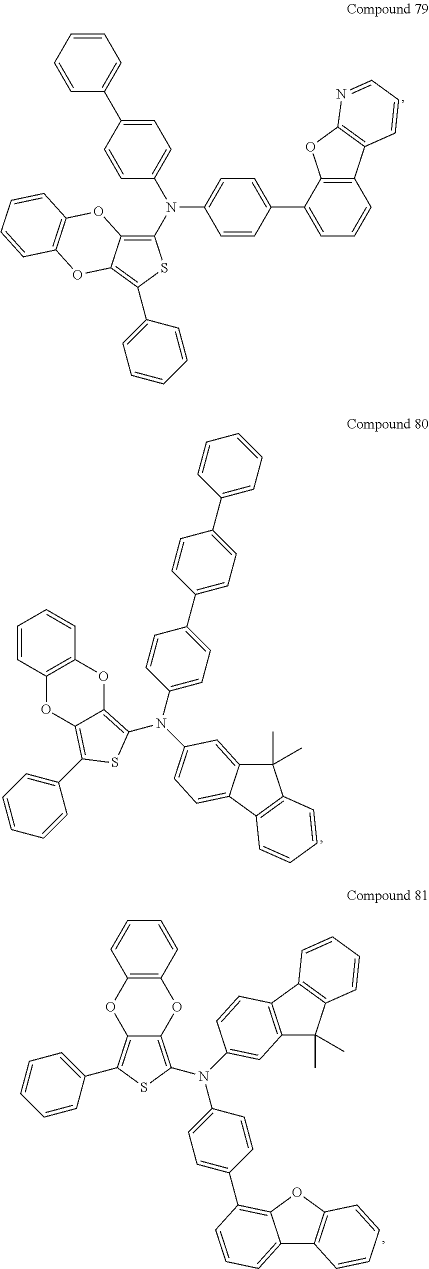 [SMILES: c1ccc(-c2ccc(N(c3ccc(-c4cccc5c4oc4ncccc45)cc3)c3sc(-c4ccccc4)c4c3Oc3ccccc3O4)cc2)cc1 ; CC1(C)c2ccccc2-c2ccc(N(c3ccc(-c4ccc(-c5ccccc5)cc4)cc3)c3sc(-c4ccccc4)c4c3Oc3ccccc3O4)cc21 ; CC1(C)c2ccccc2-c2ccc(N(c3ccc(-c4cccc5c4oc4ccccc45)cc3)c3sc(-c4ccccc4)c4c3Oc3ccccc3O4)cc21]

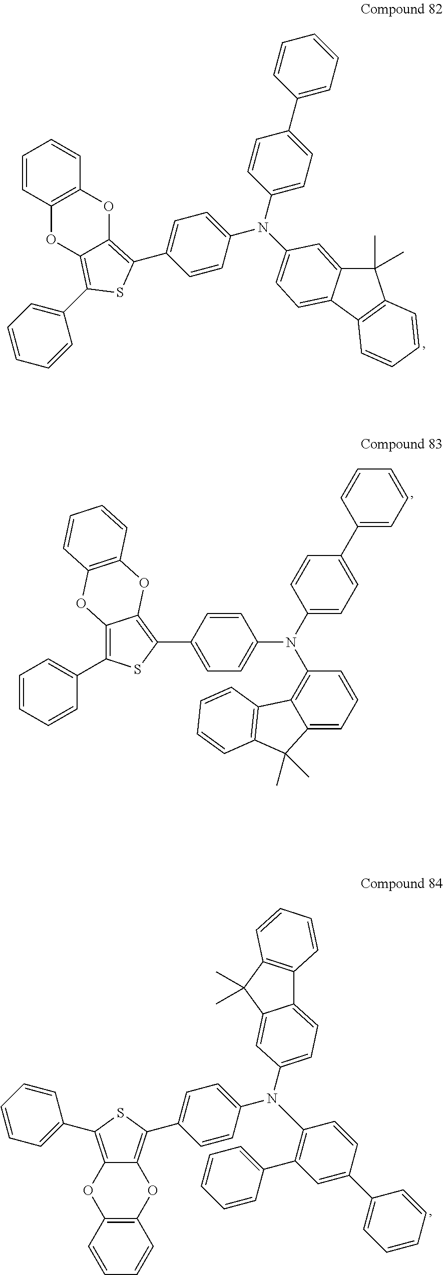 [SMILES: CC1(C)c2ccccc2-c2ccc(N(c3ccc(-c4ccccc4)cc3)c3ccc(-c4sc(-c5ccccc5)c5c4Oc4ccccc4O5)cc3)cc21 ; CC1(C)c2ccccc2-c2c(N(c3ccc(-c4ccccc4)cc3)c3ccc(-c4sc(-c5ccccc5)c5c4Oc4ccccc4O5)cc3)cccc21 ; CC1(C)c2ccccc2-c2ccc(N(c3ccc(-c4sc(-c5ccccc5)c5c4Oc4ccccc4O5)cc3)c3ccc(-c4ccccc4)cc3-c3ccccc3)cc21]

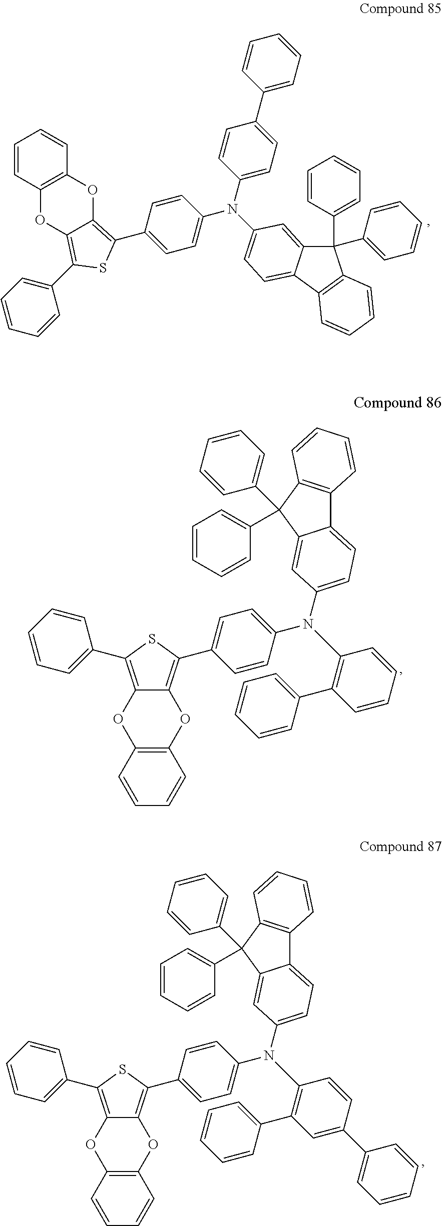 [SMILES: c1ccc(-c2ccc(N(c3ccc(-c4sc(-c5ccccc5)c5c4Oc4ccccc4O5)cc3)c3ccc4c(c3)C(c3ccccc3)(c3ccccc3)c3ccccc3-4)cc2)cc1 ; c1ccc(-c2ccccc2N(c2ccc(-c3sc(-c4ccccc4)c4c3Oc3ccccc3O4)cc2)c2ccc3c(c2)C(c2ccccc2)(c2ccccc2)c2ccccc2-3)cc1 ; c1ccc(-c2ccc(N(c3ccc(-c4sc(-c5ccccc5)c5c4Oc4ccccc4O5)cc3)c3ccc4c(c3)C(c3ccccc3)(c3ccccc3)c3ccccc3-4)c(-c3ccccc3)c2)cc1]

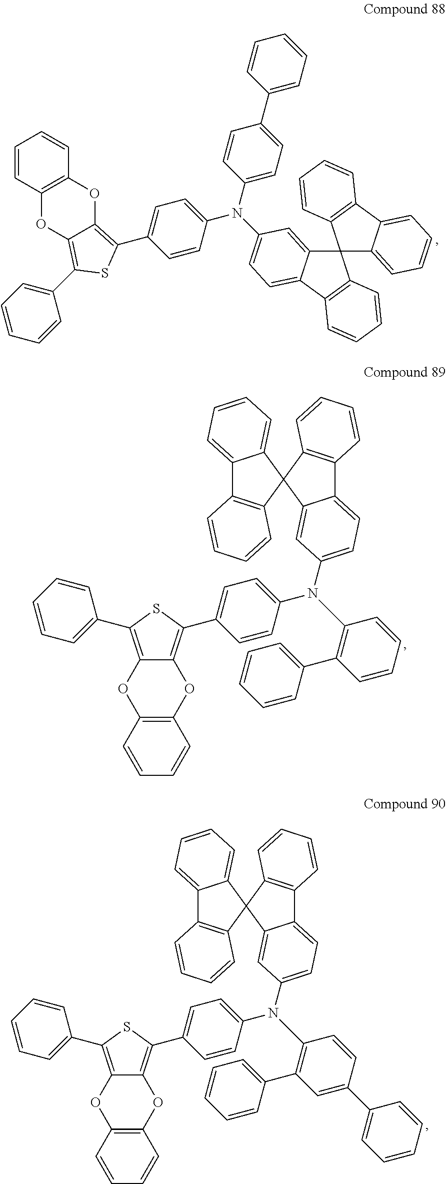 [SMILES: c1ccc(-c2ccc(N(c3ccc(-c4sc(-c5ccccc5)c5c4Oc4ccccc4O5)cc3)c3ccc4c(c3)C3(c5ccccc5-c5ccccc53)c3ccccc3-4)cc2)cc1 ; c1ccc(-c2ccccc2N(c2ccc(-c3sc(-c4ccccc4)c4c3Oc3ccccc3O4)cc2)c2ccc3c(c2)C2(c4ccccc4-c4ccccc42)c2ccccc2-3)cc1 ; c1ccc(-c2ccc(N(c3ccc(-c4sc(-c5ccccc5)c5c4Oc4ccccc4O5)cc3)c3ccc4c(c3)C3(c5ccccc5-c5ccccc53)c3ccccc3-4)c(-c3ccccc3)c2)cc1]

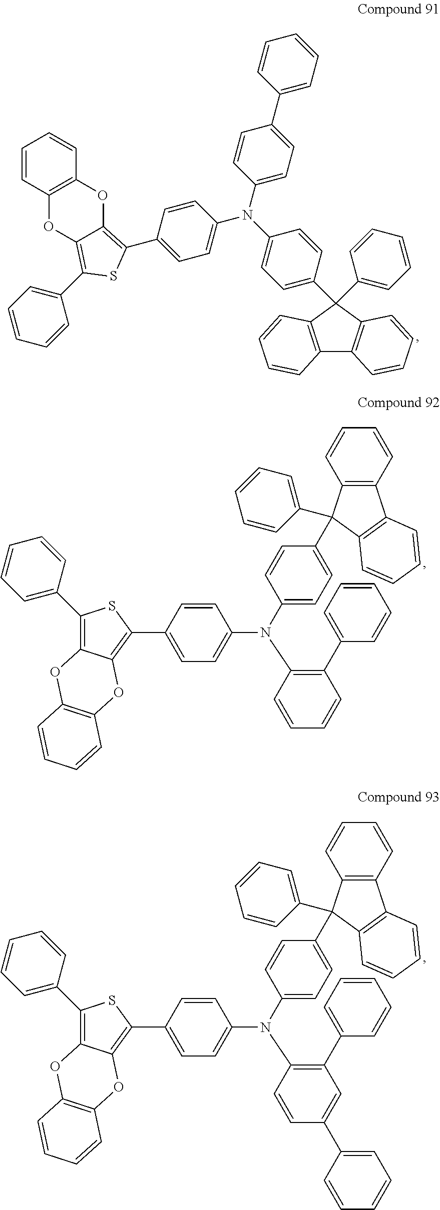 [SMILES: c1ccc(-c2ccc(N(c3ccc(-c4sc(-c5ccccc5)c5c4Oc4ccccc4O5)cc3)c3ccc(C4(c5ccccc5)c5ccccc5-c5ccccc54)cc3)cc2)cc1 ; c1ccc(-c2ccccc2N(c2ccc(-c3sc(-c4ccccc4)c4c3Oc3ccccc3O4)cc2)c2ccc(C3(c4ccccc4)c4ccccc4-c4ccccc43)cc2)cc1 ; c1ccc(-c2ccc(N(c3ccc(-c4sc(-c5ccccc5)c5c4Oc4ccccc4O5)cc3)c3ccc(C4(c5ccccc5)c5ccccc5-c5ccccc54)cc3)c(-c3ccccc3)c2)cc1]

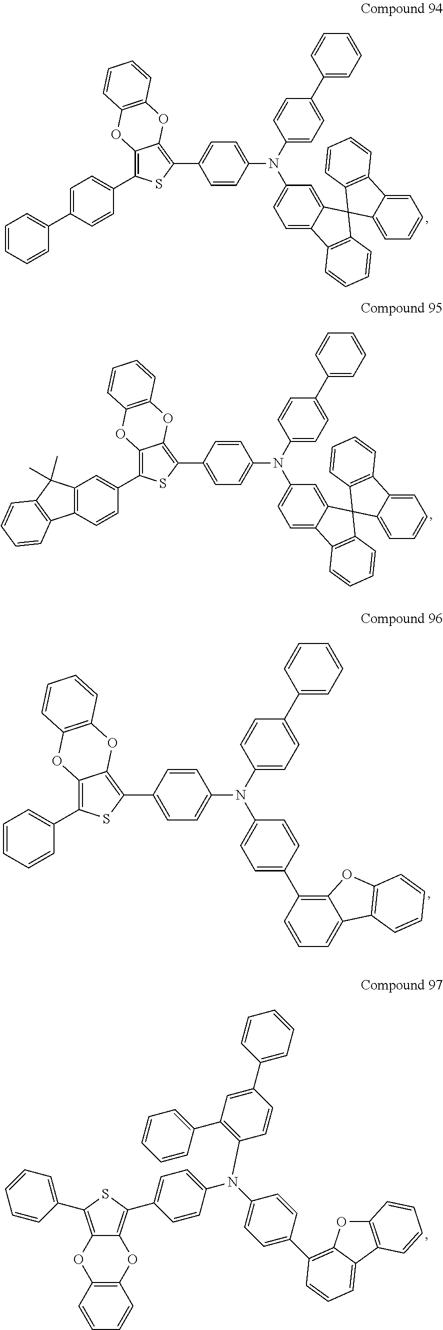 [SMILES: c1ccc(-c2ccc(-c3sc(-c4ccc(N(c5ccc(-c6ccccc6)cc5)c5ccc6c(c5)C5(c7ccccc7-c7ccccc75)c5ccccc5-6)cc4)c4c3Oc3ccccc3O4)cc2)cc1 ; CC1(C)c2ccccc2-c2ccc(-c3sc(-c4ccc(N(c5ccc(-c6ccccc6)cc5)c5ccc6c(c5)C5(c7ccccc7-c7ccccc75)c5ccccc5-6)cc4)c4c3Oc3ccccc3O4)cc21 ; c1ccc(-c2ccc(N(c3ccc(-c4sc(-c5ccccc5)c5c4Oc4ccccc4O5)cc3)c3ccc(-c4cccc5c4oc4ccccc45)cc3)cc2)cc1 ; c1ccc(-c2ccc(N(c3ccc(-c4sc(-c5ccccc5)c5c4Oc4ccccc4O5)cc3)c3ccc(-c4cccc5c4oc4ccccc45)cc3)c(-c3ccccc3)c2)cc1]

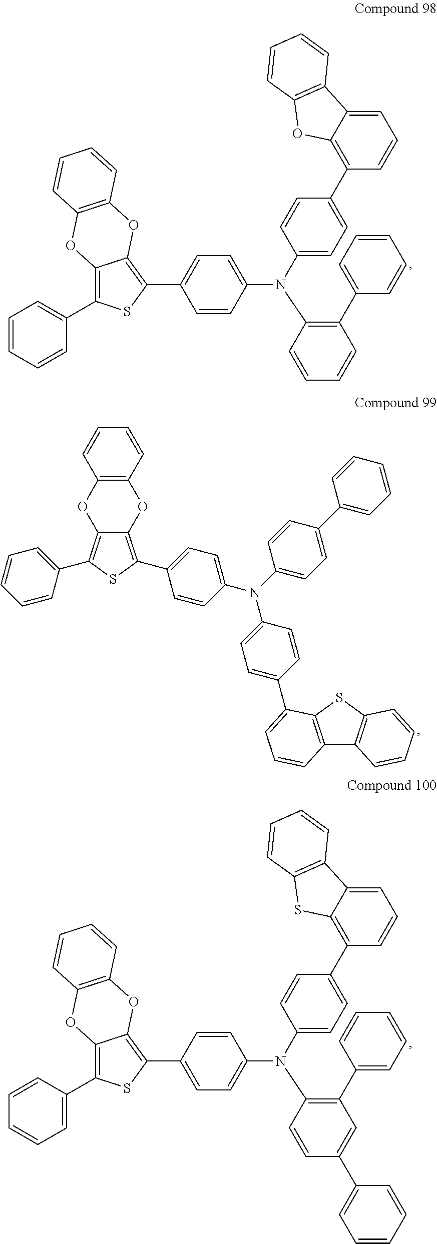

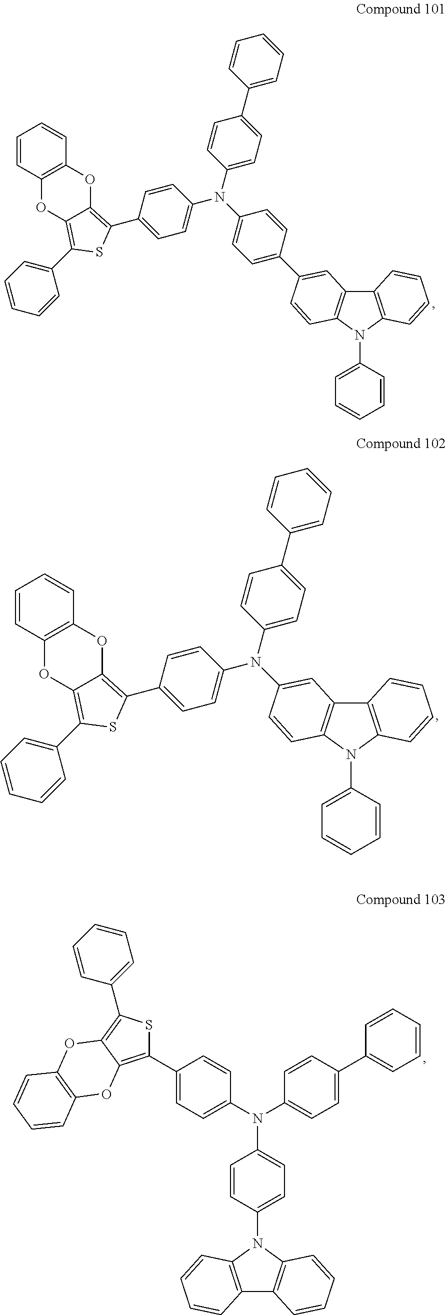 [SMILES: c1ccc(-c2ccc(N(c3ccc(-c4ccc5c(c4)c4ccccc4n5-c4ccccc4)cc3)c3ccc(-c4sc(-c5ccccc5)c5c4Oc4ccccc4O5)cc3)cc2)cc1 ; c1ccc(-c2ccc(N(c3ccc(-c4sc(-c5ccccc5)c5c4Oc4ccccc4O5)cc3)c3ccc4c(c3)c3ccccc3n4-c3ccccc3)cc2)cc1 ; c1ccc(-c2ccc(N(c3ccc(-c4sc(-c5ccccc5)c5c4Oc4ccccc4O5)cc3)c3ccc(-n4c5ccccc5c5ccccc54)cc3)cc2)cc1]

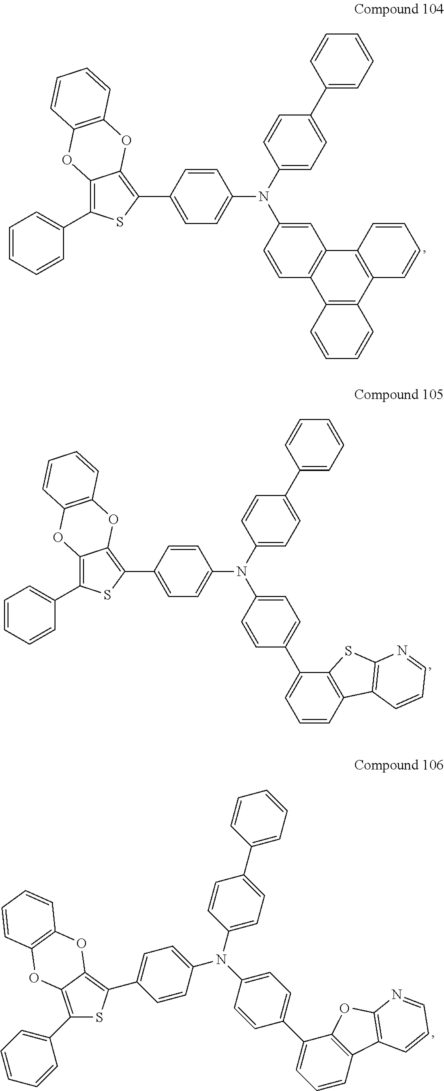 [SMILES: c1ccc(-c2ccc(N(c3ccc(-c4sc(-c5ccccc5)c5c4Oc4ccccc4O5)cc3)c3ccc4c5ccccc5c5ccccc5c4c3)cc2)cc1 ; c1ccc(-c2ccc(N(c3ccc(-c4sc(-c5ccccc5)c5c4Oc4ccccc4O5)cc3)c3ccc(-c4cccc5c4sc4ncccc45)cc3)cc2)cc1 ; c1ccc(-c2ccc(N(c3ccc(-c4sc(-c5ccccc5)c5c4Oc4ccccc4O5)cc3)c3ccc(-c4cccc5c4oc4ncccc45)cc3)cc2)cc1]

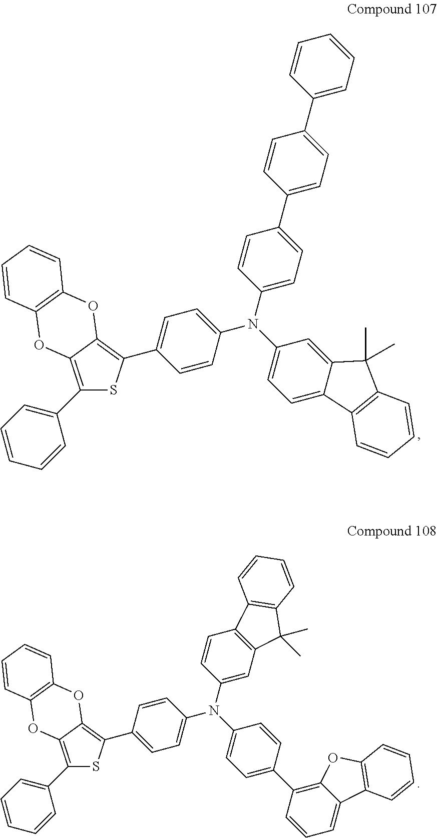 [SMILES: CC1(C)c2ccccc2-c2ccc(N(c3ccc(-c4ccc(-c5ccccc5)cc4)cc3)c3ccc(-c4sc(-c5ccccc5)c5c4Oc4ccccc4O5)cc3)cc21 ; CC1(C)c2ccccc2-c2ccc(N(c3ccc(-c4sc(-c5ccccc5)c5c4Oc4ccccc4O5)cc3)c3ccc(-c4cccc5c4oc4ccccc45)cc3)cc21]

According to another embodiment, an electroluminescent device is disclosed. The electroluminescent device comprises:

an anode,

a cathode,

a series of organic layers disposed between the anode and cathode, wherein at least one of the organic layers comprises a compound having formula 1:

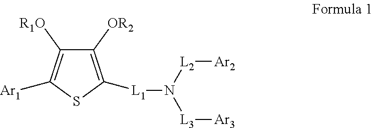

Wherein

R₁ and R₂ are each independently selected from the group consisting of a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, and combinations thereof;

R₁ and R₂ are optionally joined to form a ring;

Each of Ar₁, Ar₂, and Ar₃ are independently selected from the group consisting of a substituted or unsubstituted aryl group having 6 to 50 ring carbon atoms, a substituted or unsubstituted heteroaryl group having 5 to 50 ring atoms, and combinations thereof;

Each of L₁, L₂, and L₃ are independently selected from the group consisting of a single bond, an arylene group having 6 to 30 ring carbon atoms, a heteroarylene group having 5 to 30 ring atoms, and combinations thereof.

In one embodiment, wherein the device comprises a hole transporting layer, wherein the hole transporting layer comprises the compound having Formula 1.

In one embodiment, wherein the device comprises a hole injection layer, wherein the hole injection layer comprises the compound having Formula 1.

In one embodiment, wherein the device comprises a hole injection layer and the hole injection layer comprises the compound having Formula 1, wherein the hole injection layer further comprises a p-type conductivity dopant.

In one embodiment, wherein the device comprises a hole transporting layer and the hole transporting layer comprises the compound having Formula 1, wherein the hole transporting layer further comprises a p-type conductivity dopant.

According to yet another embodiment, a formulation comprising a compound of Formula 1 is also disclosed. The specific structure of the compound having Formula 1 is described in any of the above embodiments.

Combination with Other Materials

The materials described herein as useful for a particular layer in an organic light emitting device may be used in combination with a wide variety of other materials present in the device. The combinations of these materials are described in more detail in U.S. Pat. App. No. 20160359122 at paragraphs 0132-0161, which are incorporated by reference in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, HIL, HTL materials disclosed herein may be used in combination with a wide variety of emissive dopants, hosts, transport layers, blocking layers, injection layers, electrodes and other layers that may be present. The combination of these materials is described in detail in paragraphs 0080-0101 of U.S. Pat. App. No. 20150349273, which are incorporated by reference in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatography-mass spectrometer produced by SHIMADZU, gas chromatography-mass spectrometer produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in the present invention.

### Synthesis Example

The method for preparing the compounds of the present invention is not limited. The following compounds are exemplified as typical but non-limiting examples, and their synthesis route and preparation method are as follows:

### Synthesis Example 1: Synthesis of Compound 1

Step 1:

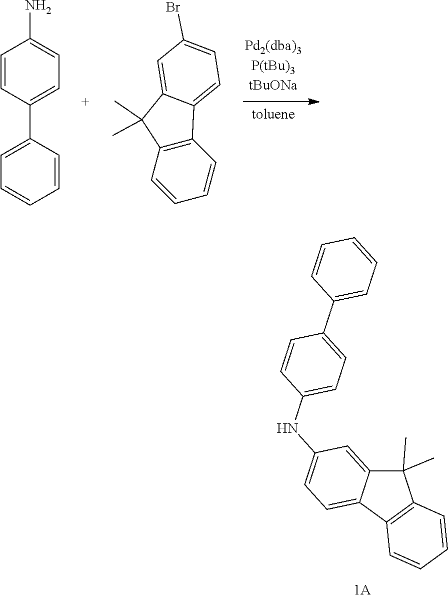 [SMILES: Nc1ccc(-c2ccccc2)cc1 ; CC1(C)c2ccccc2-c2ccc(Br)cc21 ; CC1(C)c2ccccc2-c2ccc(Nc3ccc(-c4ccccc4)cc3)cc21]

A mixture of 4-aminobiphenyl (10 g, 59.1 mmol), 9,9-dimethyl-2-bromofluorene (12.42 g, 45.45 mmol), Pd₂(dba)₃ (2.08 g, 2.3 mmol) and t-BuONa (8.7 g, 90.9 mmol) in toluene (200 mL) was degassed with N₂ for 15 min. P(t-Bu)₃ (9.1 g, 4.5 mmol, 10% in toluene) was added and the mixture was heated at 70° C. for 3 h. The reaction was cooled to room temperature, quenched with water, extracted with DCM and filtered through Celite. The filtrate was collected and separated. The organic phase was filtered through a silica-gel plug, eluting with DCM, to remove the catalyst and inorganic salts. After the solvent was removed in vacuum, a red-brown oil was obtained as crude product (24.2 g). The crude product was dissolved in DCM and purified by silica-gel column chromatography (PE:EA, 10:1) to afford a yellow solid (15 g). Recrystallization from toluene and PE yielded intermediate 1A (11.7 g, 70% yield).

Step 2:

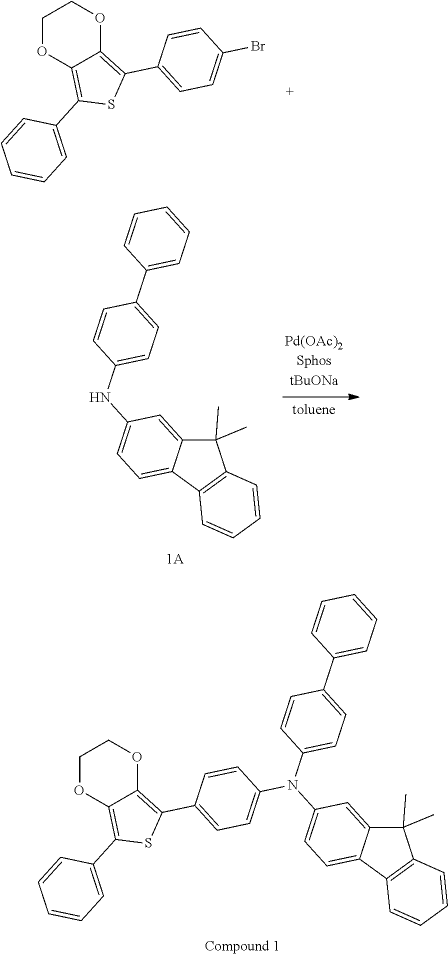 [SMILES: Brc1ccc(-c2sc(-c3ccccc3)c3c2OCCO3)cc1 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc(-c4sc(-c5ccccc5)c5c4OCCO5)cc3)cc21]

A mixture of intermediate 1A (2.68 g, 7.4 mmol), 5(4-bromophenyl)-7-phenyl-2,3-dihydrothieno[3,4-13][1,4]dioxine (2.76 g, 7.4 mmol), Pd(OAc)₂ (0.166 g, 0.74 mmol), Sphos (0.457 g, 1.11 mmol) and t-BuONa (1.42 g, 14.8 mmol) in toluene (100 mL) was degassed with N₂ for 15 min and heated to reflux for 8 h. The reaction mixture was cooled to room temperature and filtered through a silica-gel plug to remove the catalyst and inorganic salts, eluting with DCM. The solvent was removed in vacuum and a yellow oil crude product (6.3 g) was obtained. The crude product was purified by silica-gel column chromatography (PE:DCM, 10:1) to afford a yellow solid (5.5 g). The solid was refluxed in PE and filtered to afford Compound 1 (3 g, 61.8% yield) as the product. The product was confirmed as the target product, with a molecular weight of 654.

Synthesis of Compound 82

Step 1

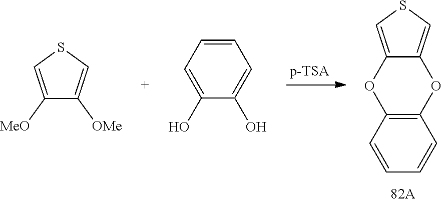 [SMILES: COc1cscc1OC ; Oc1ccccc1O ; c1ccc2c(c1)Oc1cscc1O2]

A 250 mL dry two-neck flask was charged with 3,4-dimethoxythiophene (1.44 g, 10 mmol), catechol (2.20 g, 20 mmol), p-toluenesulfonic acid monohydrate (285 mg, 1.5 mmol) and toluene (40 mL) under nitrogen, and the resulting mixture was refluxed for 12 h. After cooling to room temperature, 50 mL water was added and the mixture was extracted with ethyl acetate and aqueous NaHCO₃ solution. The combined organic layer was dried over MgSO₄, filtered and evaporated. Purification of the residue by column chromatography on silica gel (PE:EA, 2:1) afforded intermediate 82A as a white solid (420 mg, 22% yield).

Step 2

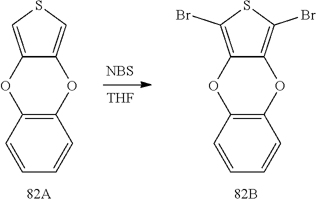

A 250 mL dry flask was charged with intermediate 82A (3.20 g, 16.8 mmol) and THF (50 mL) and was cooled down to 0° C. Then N-bromobutanimide (7.48 g, 42.05 mmol) was slowly added to the solution. The mixture was stirred at 0° C. for 1 hour and at room temperature overnight. After evaporation of the solvent, the residue was extracted with ethyl acetate, and dried over MgSO₄. The crude product was purified by column chromatography (elution with PE:EA, 3:1) to afford intermediate 82B as a white solid (5.30 g, 91% yield).

Step 3:

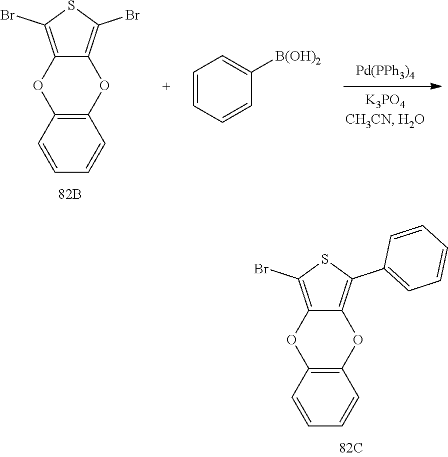

To a 100 mL two-neck flask containing a mixture of intermediate 82B (2.4 g, 6.89 mmol), phenylboronic acid (840 mg, 6.89 mmol), potassium phosphate (1.9 g, 13.78 mmol) and Pd(PPh₃)₄ (398 mg, 0.34 mmol) was added acetonitrile (15 mL) and water (5 mL) under nitrogen. The mixture was refluxed at 70° C. for 8 h. After cooling to room temperature, the mixture was extracted with DCM/water. The combined organic layer was dried over MgSO₄, filtered and evaporated. Purification of the residue by column chromatography on silica gel (elution with PE:EA, 3:1) afforded intermediate 82C as a white solid (1.3 g, 55% yield).

Step 4:

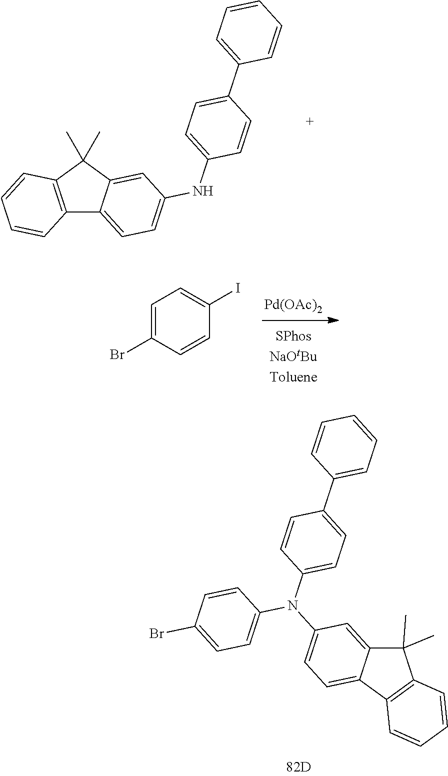

A 500 mL dry two-neck flask was charged with intermediate 1A (11.2 g, 31.11 mmol), 1-bromo-4-iodobenzene(13.2 g, 46.66 mmol), sodium tert-butoxide (7.5 g, 77.78 mmol), Sphos (1276 mg, 3.11 mmol), Pd(OAc)₂ (350 mg, 1.56 mmol) and toluene (150 mL) under nitrogen, and the resulting mixture was heated to 90° C. for 18 h. After cooling to room temperature, 50 mL water was added and the mixture was extracted with DCM/H₂O. The combined organic layer was dried over MgSO₄, filtered and evaporated. Purification of the residue by column chromatography on silica gel (elution with PE:DCM, 5:1) afforded intermediate 82D as a white solid (10.6 g, 64% yield).

Step 5:

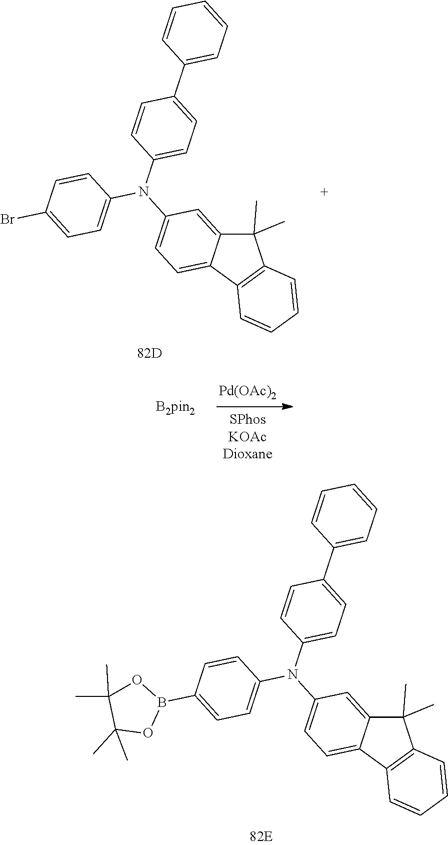

To a 100 mL dried two-neck flask containing a mixture of intermediate 82D (4.55 g, 8.82 mmol), bis(pinacolato)diboron (3.36 g, 13.23 mmol), potassium acetate (2.66 g, 22.05 mmol), Sphos (361 mg, 0.88 mmol) and Pd(OAc)₂ (100 mg, 0.44 mmol) was added 1,4-dioxane (40 mL) under nitrogen. The mixture was refluxed at 105° C. for 18 h. After evaporation of the solvent, the residue was extracted with DCM/water. The combined organic layer was dried over MgSO₄, filtered and evaporated. Purification of the crude product by column chromatography on silica gel (elution with PE:EA, 2:1) followed by recrystallization from toluene afforded intermediate 82E as a white solid (2.83 g, 57% yield).

Step 6:

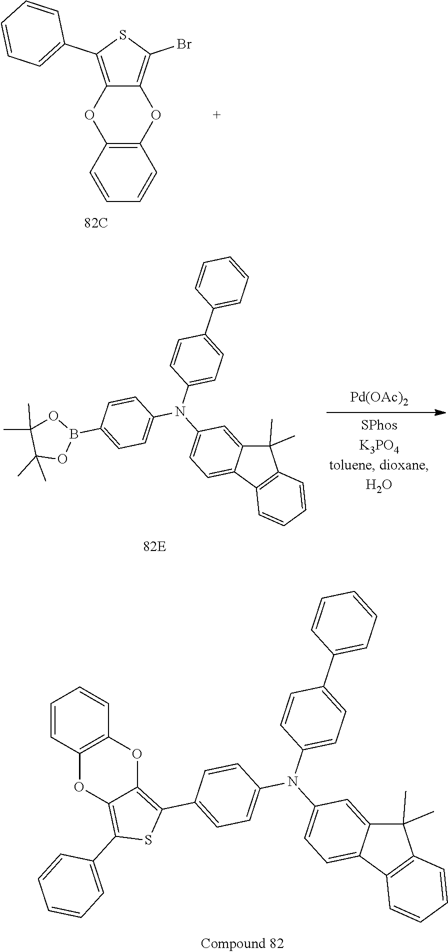 [SMILES: Brc1sc(-c2ccccc2)c2c1Oc1ccccc1O2 ; CC1(C)c2ccccc2-c2ccc(N(c3ccc(B4OC(C)(C)C(C)(C)O4)cc3)c3ccc(-c4ccccc4)cc3)cc21 ; "SPhosK3PO4toluene, dioxane,H2O" ; CC1(C)c2ccccc2-c2ccc(N(c3ccc(-c4ccccc4)cc3)c3ccc(-c4sc(-c5ccccc5)c5c4Oc4ccccc4O5)cc3)cc21 ; "Pd(OAc)2"]

To a 100 mL two-neck flask containing a mixture of intermediate 82C (1.73 g, 5.0 mmol), intermediate 82E (2.82 g, 5.0 mmol), potassium phosphate (2.66 g, 10.0 mmol), Sphos (205 mg, 0.5 mmol) and Pd(OAc)₂ (56 mg, 0.25 mmol) was added dried toluene (20 mL), 1,4-dioxane (5 mL) and water (5 mL) under nitrogen. The mixture was refluxed at 100° C. for 12 h. After cooling to room temperature, the mixture was extracted with DCM/water. The combined organic layer was dried over MgSO₄, filtered and evaporated. Purification of the residue by column chromatography on silica gel (elution with PE:DCM, 2:1) followed by recrystallization from toluene afforded Compound 82 as a yellow solid (2.49 g, 70% yield). The product was confirmed as the target product, with a molecular weight of 702.

The persons skilled in the art should know that the above preparation method is only an illustrative example, and the persons skilled in the art can obtain the structure of other compounds of the present invention by modifying the above preparation method.

### Device Example 1

A glass substrate with 80 nm thick indium-tin-oxide (ITO) anode was first cleaned and then treated with oxygen plasma and UV ozone. After the treatments, the substrate was baked in a glovebox to remove moisture. The substrate was then mounted on a substrate holder and loaded into a vacuum chamber. The organic layers specified below were deposited in sequence by thermal vacuum deposition on the ITO anode at a rate of 0.2-2 Å/s at a vacuum level of around 10⁻⁸ torr. Compound HI was used as the hole injection layer (HIL). Compound 82 was used as the first hole transporting layer (HTL1) and second hole transporting layer (HTL2). Then Compound H1 was used as the electron blocking layer (EBL). Then Compound RD was doped in Compound H2 as the emitting layer (EML). Then Compound ET and 8-hydroxyquinolinolato-lithium (Liq) were co-deposited as the electron transporting layer (ETL). Finally, 1 nm of Liq was deposited as the electron injection layer and 120 nm of Al was deposited as the cathode. The device was then transferred back to the glovebox and encapsulated with a glass lid and a moisture getter.

### Device Example 2

A glass substrate with 80 nm thick indium-tin-oxide (ITO) anode was first cleaned and then treated with oxygen plasma and UV ozone. After the treatments, the substrate was baked in a glovebox to remove moisture. The substrate was then mounted on a substrate holder and loaded into a vacuum chamber. The organic layers specified below were deposited in sequence by thermal vacuum deposition on the ITO anode at a rate of 0.2-2 Å/s at a vacuum level of around 10⁻⁸ torr. Compound X was doped in Compound 82 as the hole injection layer (HIL) and the first hole transporting layer (HTL1). Compound 82 was used as the second hole transporting layer (HTL2). Then Compound H1 was used as the electron blocking layer (EBL). Then Compound RD was doped in Compound H2 as the emitting layer (EML). Then Compound ET and 8-hydroxyquinolinolato-lithium(Liq) were co-deposited as the electron transporting layer (ETL). Finally, 1 nm of Liq was deposited as the electron injection layer and 120 nm of Al was deposited as the cathode. The device was then transferred back to the glovebox and encapsulated with a glass lid and a moisture getter.

### Device Example 3

Device example 3 was fabricated in the same manner as Device example 2, except that the deposition ratio between Compound X and Compound 82 is different.

### Device Example 4

Device example 4 was fabricated in the same manner as Device example 2, except that Compound X and Compound 1 were used as the hole injection layer (HIL) and the first hole transporting layer (HTL1), and Compound 1 was used as the second hole transporting layer (HTL2).

### Device Example 5

Device example 5 was fabricated in the same manner as Device example 4, except that the deposition ratio between Compound X and Compound 1 is different.

### Comparative Example 1

Comparative example 1 was fabricated in the same manner as Device example 1, except that Compound HT were used as the first hole transporting layer (HTL1) and the second hole transporting layer (HTL2).

### Comparative Example 2

Comparative example 2 was fabricated in the same manner as Device example 2, except that Compound X and Compound HT were used as the hole injection layer (HIL) and the first hole transporting layer (HTL1), and Compound HT was used as the second hole transporting layer.

### Comparative Example 3

Comparative example 3 was fabricated in the same manner as Comparative example 2, except that the deposition ratio between Compound X and Compound HT is different.

The detailed part structure and thicknesses of the device layers are shown in the table below, wherein the codename of the compounds used in each layer are recorded. In the layers in which more than one material were used, they were obtained by doping different compounds in the weight ratios described therein.

TABLE 1
Device structure of device examples

| Device ID | HIL | HTL1 | HTL2 | EBL | EML | ETL |
|---|---|---|---|---|---|---|
| Example 1 | HI | 82 | 82 | H1 | H2:RD (97:3) | ET:Liq |
| | (100 Å) | (200 Å) | (200 Å) | (50 Å) | (400 Å) | (60:40) (350 Å) |
| Example 2 | X:82 | X:82 | 82 | H1 | H2:RD (97:3) | ET:Liq |
| | (3:97)(100 Å) | (3:97)(200 Å) | (200 Å) | (50 Å) | (400 Å) | (60:40) (350 Å) |
| Example 3 | X:82 | X:82 | 82 | H1 | H2:RD (97:3) | ET:Liq |
| | (6:94)(100 Å) | (6:94)(200 Å) | (200 Å) | (50 Å) | (400 Å) | (60:40) (350 Å) |
| Example 4 | X:1 (3:97)(100 Å) | X:1 | 1 | H1 | H2:RD (97:3) | ET:Liq |
| | | (3:97)(200 Å) | (200 Å) | (50 Å) | (400 Å) | (60:40) (350 Å) |
| Example 5 | X:1 (6:94)(100 Å) | X:1 | 1 | H1 | H2:RD (97:3) | ET:Liq |
| | | (6:94)(200 Å) | (200 Å) | (50 Å) | (400 Å) | (60:40) (350 Å) |
| Comparative | HI | HT | HT | H1 | H2:RD (97:3) | ET:Liq |
| example 1 | (100 Å) | (200 Å) | (200 Å) | (50 Å) | (400 Å) | (60:40) (350 Å) |
| Comparative | X:HT | X:HT | HT | H1 | H2:RD (97:3) | ET:Liq |
| example 2 | (3:97)(100 Å) | (3:97)(200 Å) | (200 Å) | (50 Å) | (400 Å) | (60:40) (350 Å) |
| Comparative | X:HT | X:HT | HT | H1 | H2:RD (97:3) | ET:Liq |
| example 3 | (6:94)(100 Å) | (6:94)(200 Å) | (200 Å) | (50 Å) | (400 Å) | (60:40) (350 Å) |

The structures of the materials used in the devices are shown below

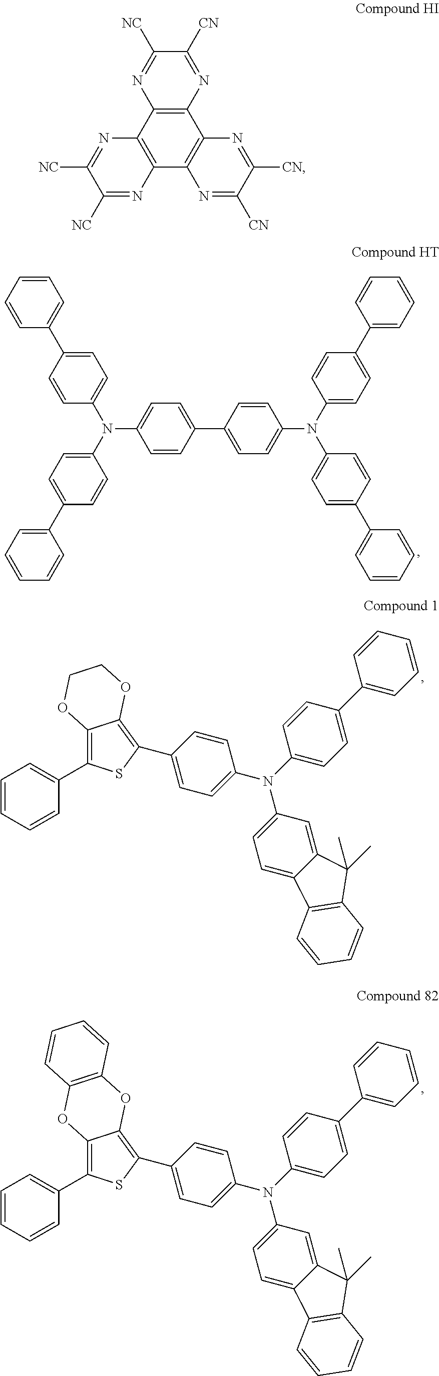 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; CC1(C)c2ccccc2-c2ccc(N(c3ccc(-c4ccccc4)cc3)c3ccc(-c4sc(-c5ccccc5)c5c4OCCO5)cc3)cc21 ; CC1(C)c2ccccc2-c2ccc(N(c3ccc(-c4ccccc4)cc3)c3ccc(-c4sc(-c5ccccc5)c5c4Oc4ccccc4O5)cc3)cc21]

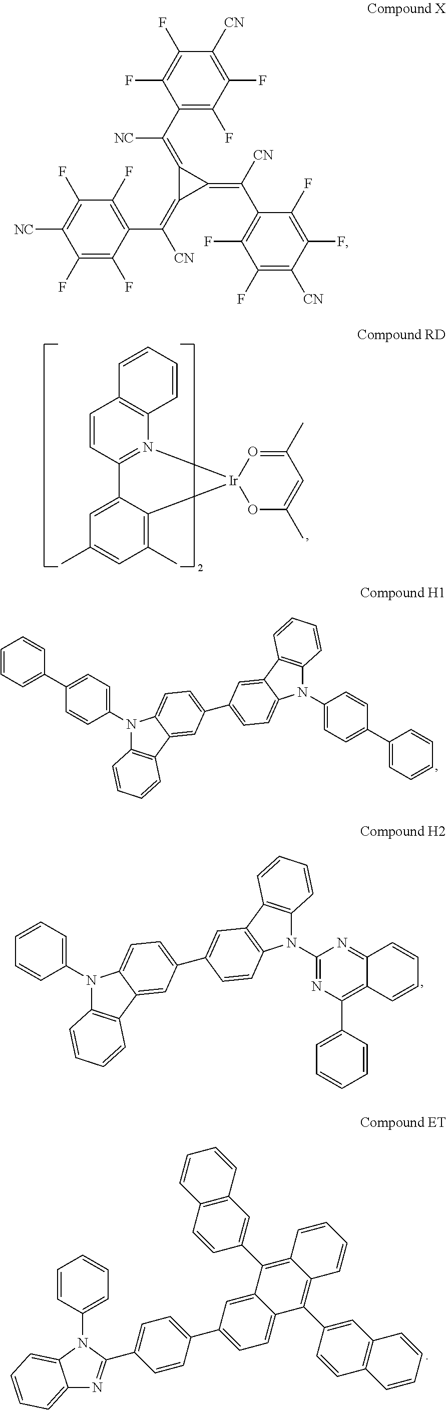

The IVL characteristics of the devices were measured. Table 2 shows the luminance efficiency (LE), power efficiency (PE), λmax, full width at half maximum (FWHM), voltage (V) and CIE data at 1,000 cd/m².

TABLE 2
Device data

| | | λmax | FWHM | Voltage | LE | PE |
|---|---|---|---|---|---|---|
| Device ID | CIE (x, y) | (nm) | (nm) | (V) | (cd/A) | (lm/W) |
| Example 1 | 0.663, 0.336 | 620 | 62.8 | 3.86 | 21.58 | 17.58 |
| Example 2 | 0.664, 0.335 | 621 | 63.1 | 3.83 | 21.01 | 17.21 |
| Example 3 | 0.664, 0.335 | 621 | 63.3 | 3.96 | 20.86 | 16.54 |
| Example 4 | 0.663, 0.335 | 620 | 63.5 | 3.98 | 20.64 | 16.28 |
| Example 5 | 0.663, 0.336 | 620 | 63.7 | 4.04 | 20.25 | 15.75 |
| Comparative | 0.664, 0.335 | 620 | 62.1 | 3.80 | 22.02 | 18.22 |
| example 1 | | | | | | |
| Comparative | 0.664, 0.335 | 619 | 62.5 | 3.82 | 20.49 | 16.83 |
| example 2 | | | | | | |
| Comparative | 0.664, 0.335 | 620 | 62.7 | 3.87 | 21.39 | 17.37 |
| example 3 | | | | | | |

Discussion:

As shown in Table 2, compared to the comparative examples 1-3 using representative HTL, HIL materials of the art, the device examples 1-5 using the compounds of the present invention as the HTL, HIL materials have the considerable performance, and it demonstrates that the thiophene-containing triarylamine compounds of the present invention can also offer excellent performance in OLED device, such as lower driving voltage and higher efficiency. And even compared to the comparative example 2, under the condition that the voltage is basically equal, the device example 2 has a higher luminance efficiency (LE) and a higher power efficiency (PE). The results above demonstrate the good prospects of the compounds disclosed in the present invention for use as the hole transporting materials and hole injection materials.

It is understood that the various embodiments described herein are by way of example only, and are not intended to limit the scope of the invention. The present invention as claimed may therefore include variations from the particular examples and preferred embodiments described herein, as will be apparent to one of skill in the art. Many of the materials and structures described herein may be substituted with other materials and structures without deviating from the spirit of the invention. It is understood that various theories as to why the invention works are not intended to be limiting.

What is claimed is:

1. A compound of Formula 1: 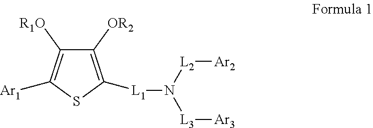
wherein,
R₁ and R₂ are each independently selected from the group consisting of a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, and combinations thereof;
R₁ and R₂ are optionally joined to form a ring;
each of Ar₁, Ar₂, and Ar₃ are independently selected from the group consisting of a substituted or unsubstituted aryl group having 6 to 50 ring carbon atoms, a substituted or unsubstituted heteroaryl group having 5 to 50 ring atoms, and combinations thereof;
each of L₁, L₂, and L₃ are independently selected from the group consisting of a single bond, an arylene group having 6 to 30 ring carbon atoms, a heteroarylene group having 5 to 30 ring atoms, and combinations thereof.

2. The compound of claim 1, wherein each of Ar₁, Ar₂, and Ar₃ are independently selected from the group consisting of: 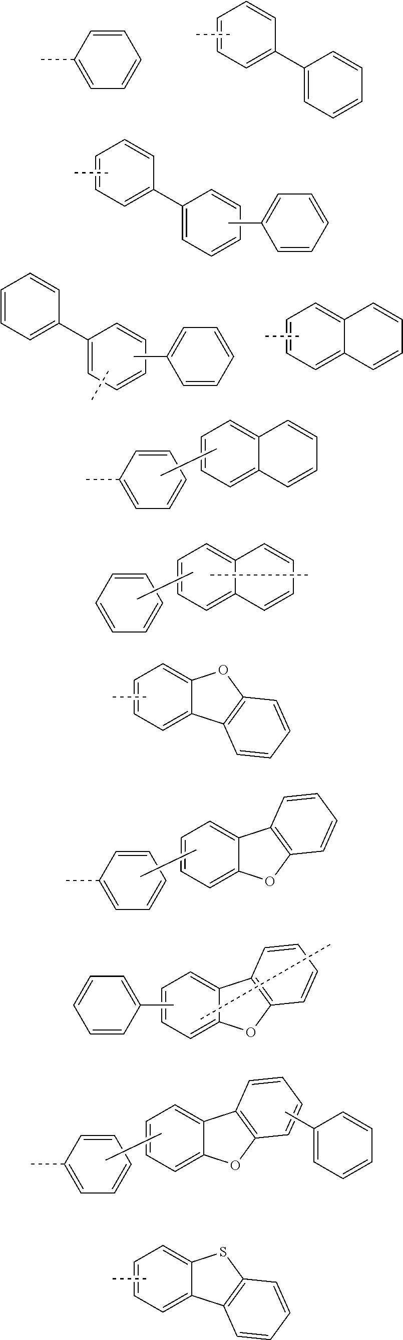 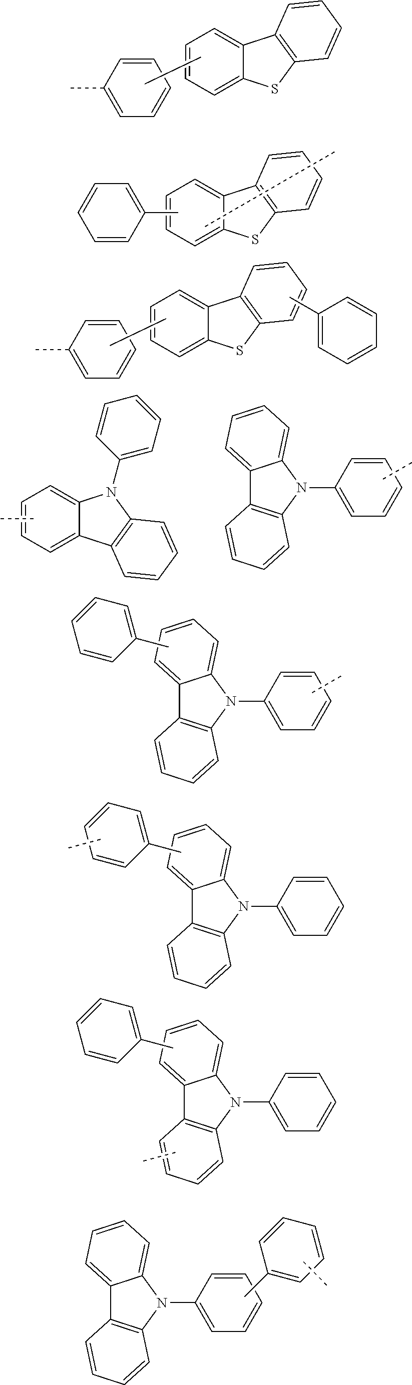 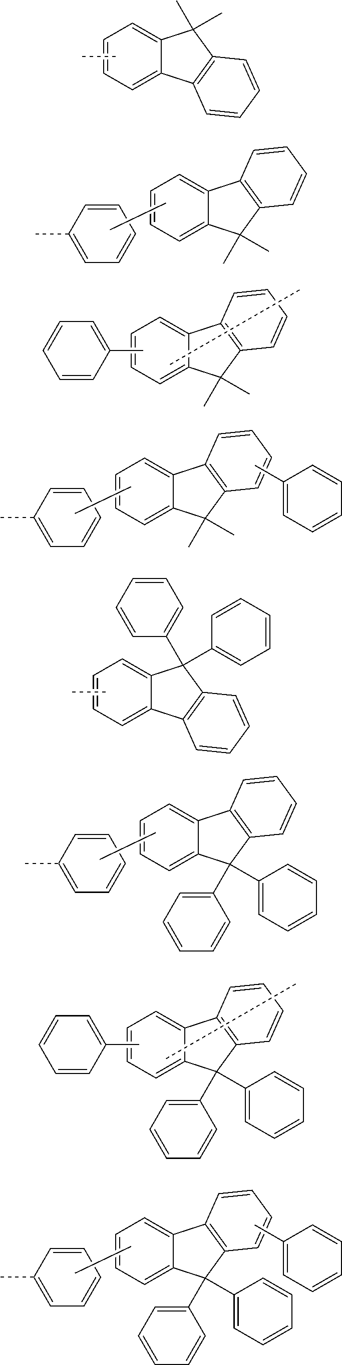 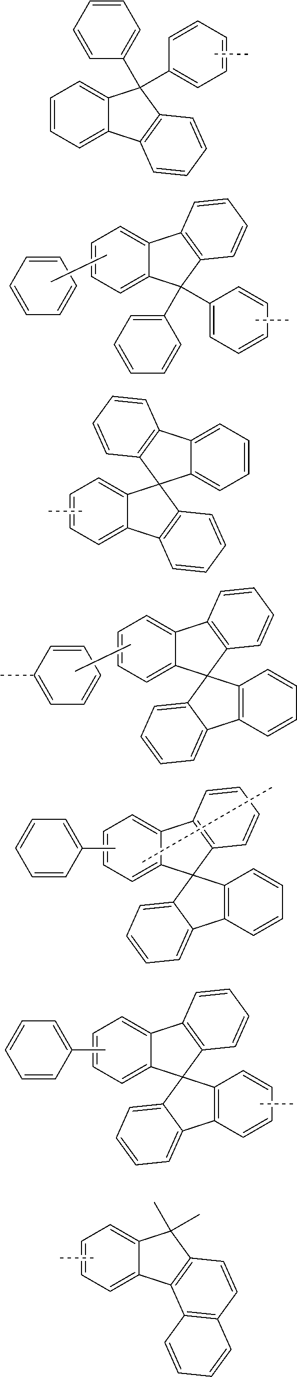 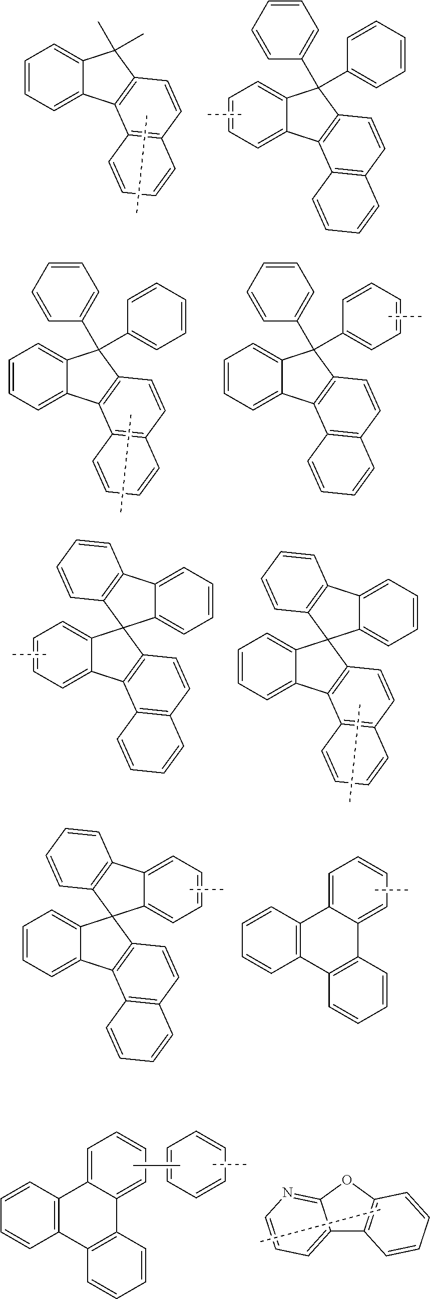 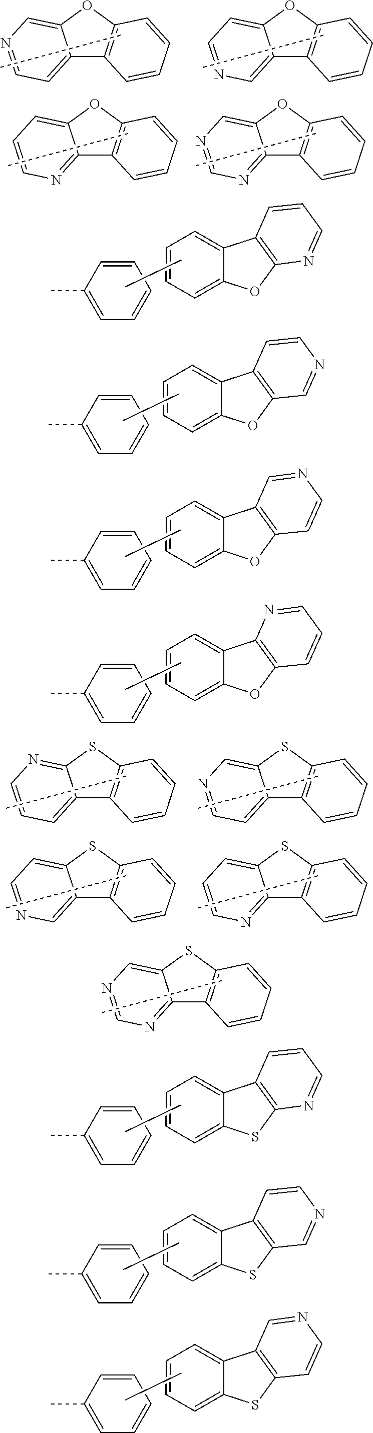
and combinations thereof.

3. The compound of claim 1, wherein each of L₁, L₂, and L₃ are independently selected from the group consisting of: 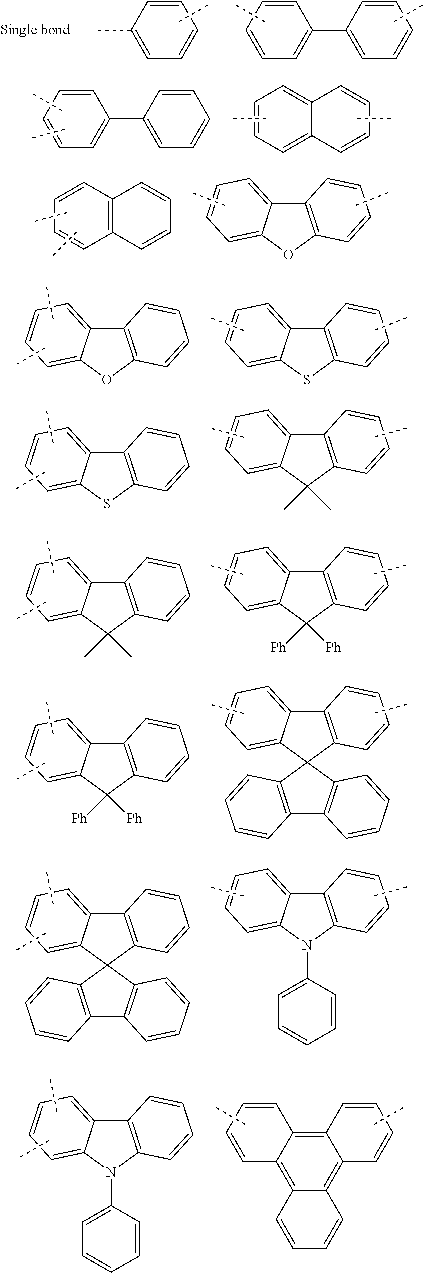 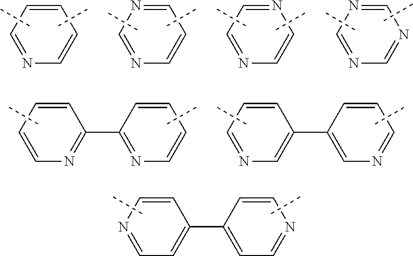
and combinations thereof.

4. The compound of claim 1, wherein the compound has the structure represented by Formula 2 or Formula 3: 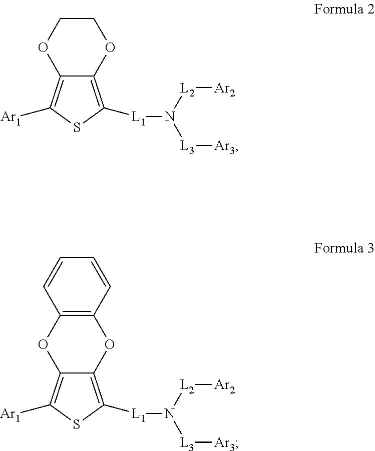
in Formula 2 or Formula 3,
each of Ar₁, Ar₂, and Ar₃ are independently selected from the group consisting of a substituted or unsubstituted aryl group having 6 to 50 ring carbon atoms, a substituted or unsubstituted heteroaryl group having 5 to 50 ring atoms, and combinations thereof;
each of L₁, L₂, and L₃ are independently selected from the group consisting of a single bond, an arylene group having 6 to 30 ring carbon atoms, a heteroarylene group having 5 to 30 ring atoms, and combinations thereof.

5. The compound of claim 1, wherein Ar₁ and Ar₂ are each independently selected from phenyl, biphenyl, or fluorene; and/or Ar₃ are selected from phenyl, biphenyl, fluorene, dibenzofuran, triphenylene, carbazole, terphenyl, dibenzothiophene, azadibenzofuran, azadibenzothiophene, or spirobifluorene.

6. The compound of claim 1, wherein each of L₁, L₂, and L₃ are independently selected from single bond, or phenylene.

7. The compound of claim 1, wherein the compound is selected from the group consisting of:

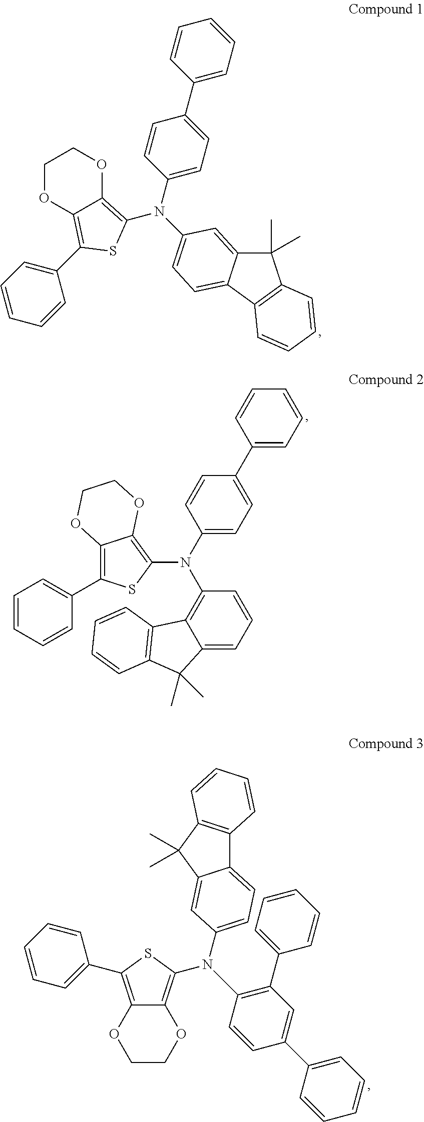

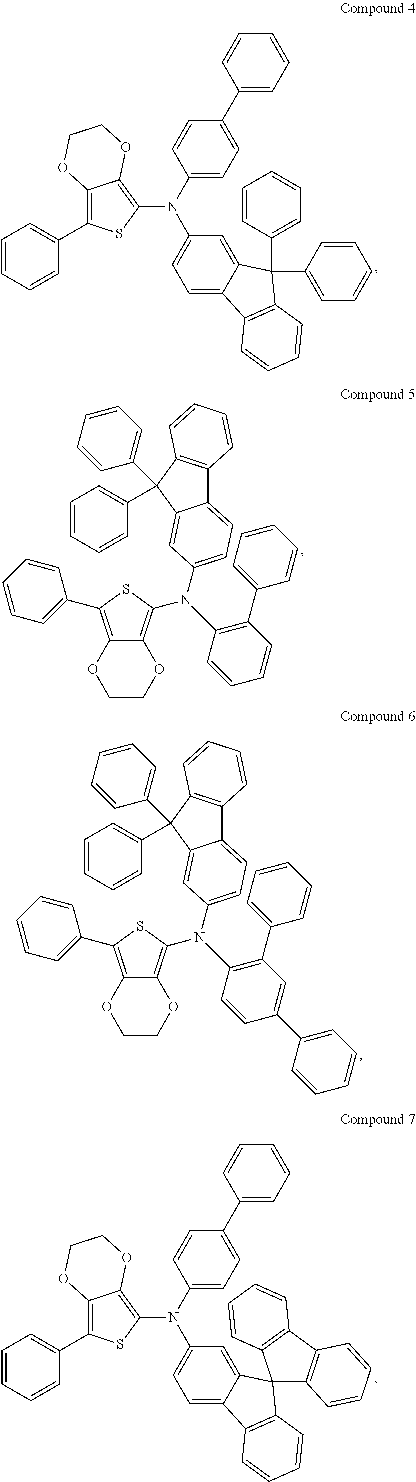 [SMILES: c1ccc(-c2ccc(N(c3ccc4c(c3)C(c3ccccc3)(c3ccccc3)c3ccccc3-4)c3sc(-c4ccccc4)c4c3OCCO4)cc2)cc1 ; c1ccc(-c2ccccc2N(c2ccc3c(c2)C(c2ccccc2)(c2ccccc2)c2ccccc2-3)c2sc(-c3ccccc3)c3c2OCCO3)cc1 ; c1ccc(-c2ccc(N(c3ccc4c(c3)C(c3ccccc3)(c3ccccc3)c3ccccc3-4)c3sc(-c4ccccc4)c4c3OCCO4)c(-c3ccccc3)c2)cc1 ; c1ccc(-c2ccc(N(c3ccc4c(c3)C3(c5ccccc5-c5ccccc53)c3ccccc3-4)c3sc(-c4ccccc4)c4c3OCCO4)cc2)cc1]

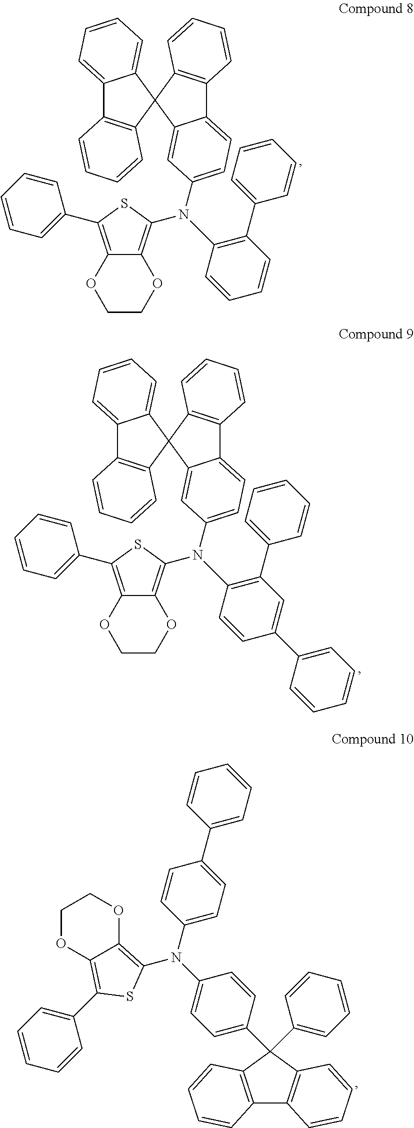 [SMILES: c1ccc(-c2ccccc2N(c2ccc3c(c2)C2(c4ccccc4-c4ccccc42)c2ccccc2-3)c2sc(-c3ccccc3)c3c2OCCO3)cc1 ; c1ccc(-c2ccc(N(c3ccc4c(c3)C3(c5ccccc5-c5ccccc53)c3ccccc3-4)c3sc(-c4ccccc4)c4c3OCCO4)c(-c3ccccc3)c2)cc1 ; c1ccc(-c2ccc(N(c3ccc(C4(c5ccccc5)c5ccccc5-c5ccccc54)cc3)c3sc(-c4ccccc4)c4c3OCCO4)cc2)cc1]

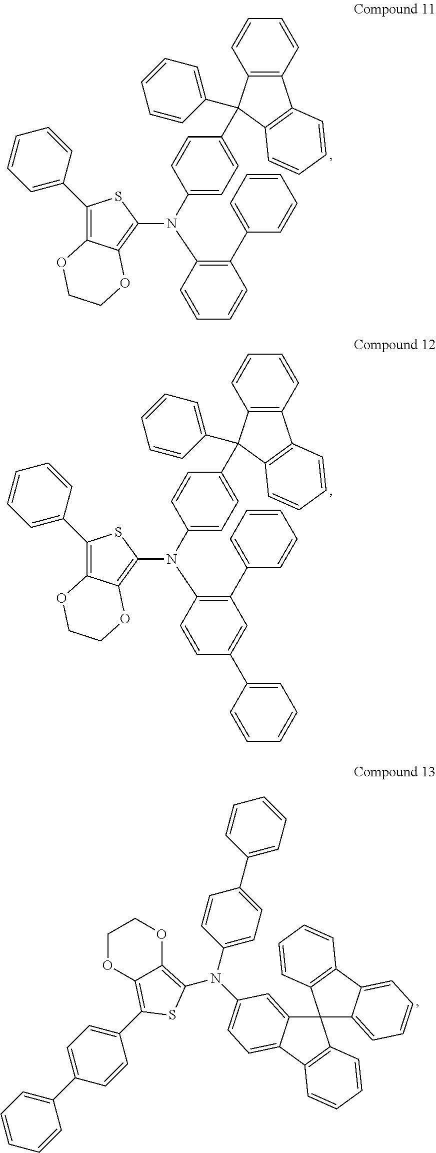

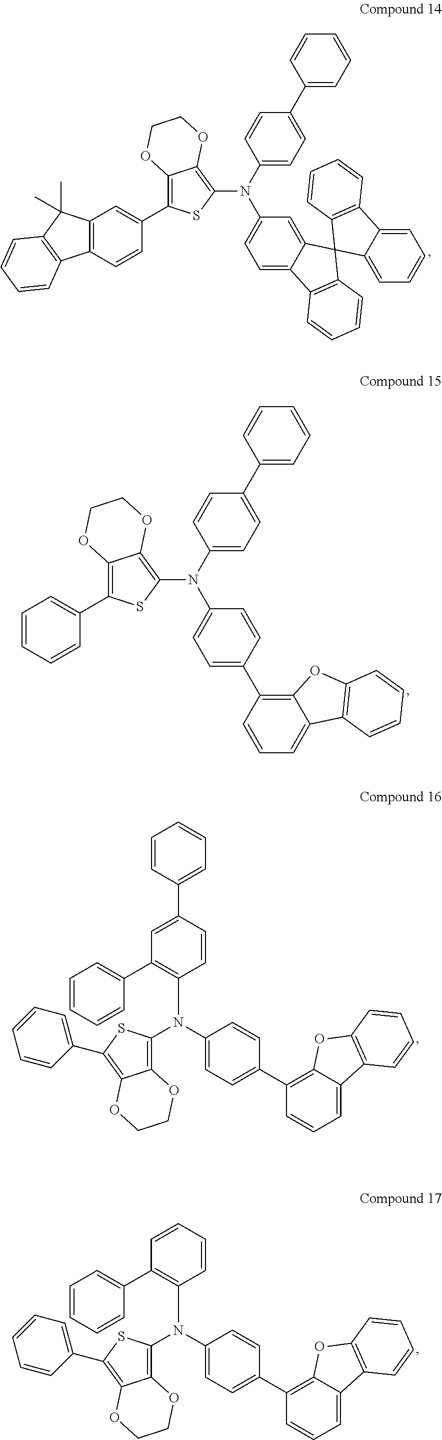 [SMILES: CC1(C)c2ccccc2-c2ccc(-c3sc(N(c4ccc(-c5ccccc5)cc4)c4ccc5c(c4)C4(c6ccccc6-c6ccccc64)c4ccccc4-5)c4c3OCCO4)cc21 ; c1ccc(-c2ccc(N(c3ccc(-c4cccc5c4oc4ccccc45)cc3)c3sc(-c4ccccc4)c4c3OCCO4)cc2)cc1 ; c1ccc(-c2ccc(N(c3ccc(-c4cccc5c4oc4ccccc45)cc3)c3sc(-c4ccccc4)c4c3OCCO4)c(-c3ccccc3)c2)cc1 ; c1ccc(-c2ccccc2N(c2ccc(-c3cccc4c3oc3ccccc34)cc2)c2sc(-c3ccccc3)c3c2OCCO3)cc1]

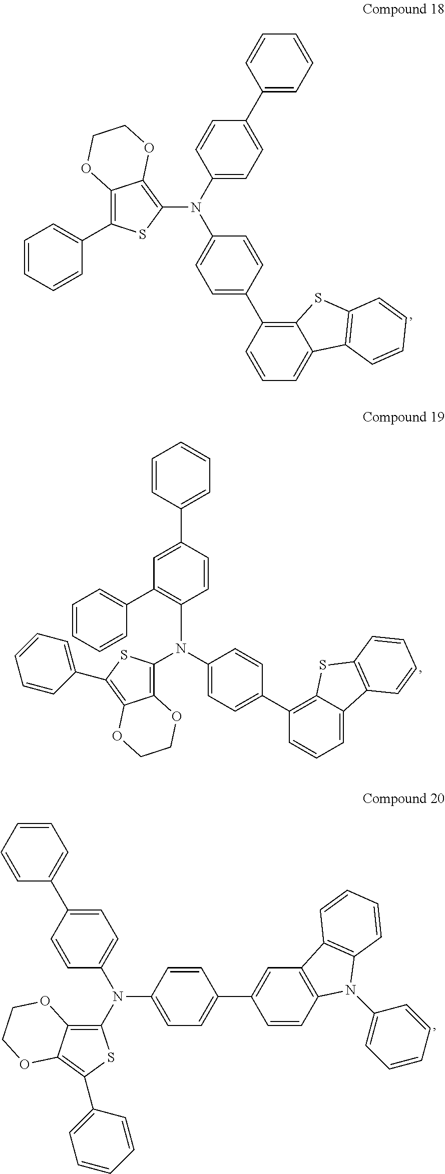 [SMILES: c1ccc(-c2ccc(N(c3ccc(-c4cccc5c4sc4ccccc45)cc3)c3sc(-c4ccccc4)c4c3OCCO4)cc2)cc1 ; c1ccc(-c2ccc(N(c3ccc(-c4cccc5c4sc4ccccc45)cc3)c3sc(-c4ccccc4)c4c3OCCO4)c(-c3ccccc3)c2)cc1 ; c1ccc(-c2ccc(N(c3ccc(-c4ccc5c(c4)c4ccccc4n5-c4ccccc4)cc3)c3sc(-c4ccccc4)c4c3OCCO4)cc2)cc1]

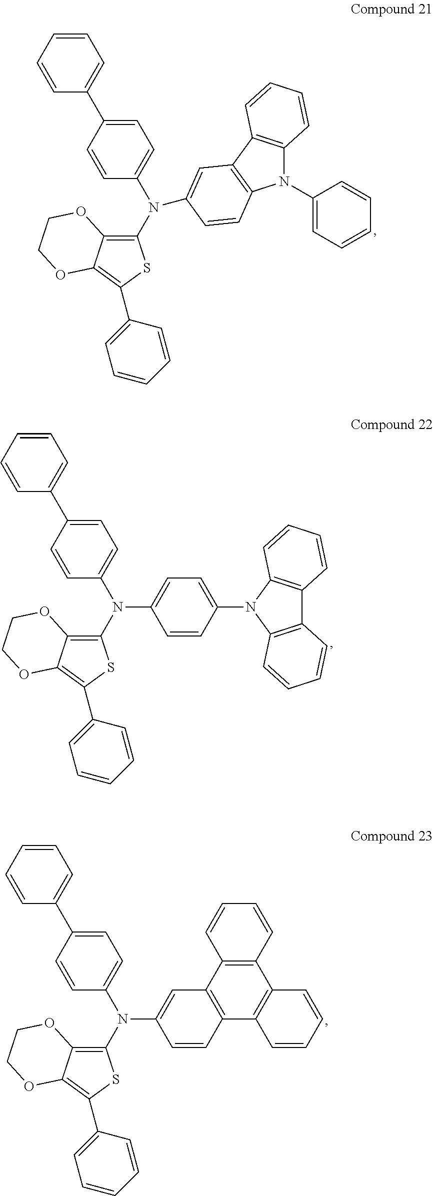 [SMILES: c1ccc(-c2ccc(N(c3ccc4c(c3)c3ccccc3n4-c3ccccc3)c3sc(-c4ccccc4)c4c3OCCO4)cc2)cc1 ; c1ccc(-c2ccc(N(c3ccc(-n4c5ccccc5c5ccccc54)cc3)c3sc(-c4ccccc4)c4c3OCCO4)cc2)cc1 ; c1ccc(-c2ccc(N(c3ccc4c5ccccc5c5ccccc5c4c3)c3sc(-c4ccccc4)c4c3OCCO4)cc2)cc1]

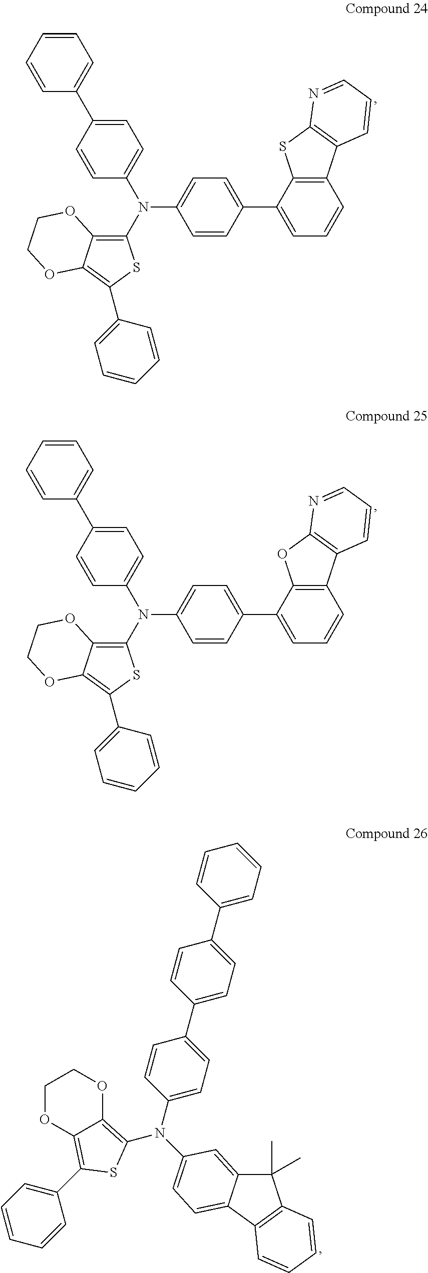 [SMILES: c1ccc(-c2ccc(N(c3ccc(-c4cccc5c4sc4ncccc45)cc3)c3sc(-c4ccccc4)c4c3OCCO4)cc2)cc1 ; c1ccc(-c2ccc(N(c3ccc(-c4cccc5c4oc4ncccc45)cc3)c3sc(-c4ccccc4)c4c3OCCO4)cc2)cc1 ; CC1(C)c2ccccc2-c2ccc(N(c3ccc(-c4ccc(-c5ccccc5)cc4)cc3)c3sc(-c4ccccc4)c4c3OCCO4)cc21]

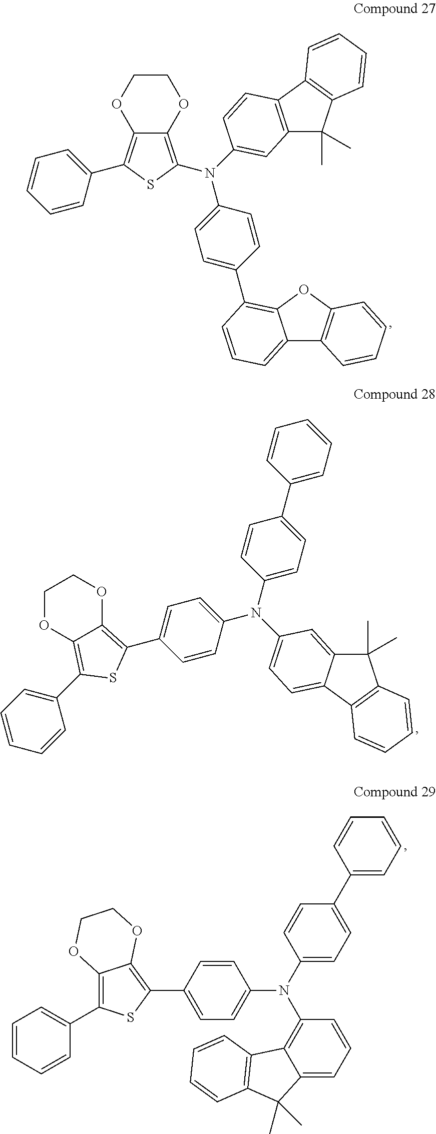 [SMILES: CC1(C)c2ccccc2-c2ccc(N(c3ccc(-c4cccc5c4oc4ccccc45)cc3)c3sc(-c4ccccc4)c4c3OCCO4)cc21 ; CC1(C)c2ccccc2-c2ccc(N(c3ccc(-c4ccccc4)cc3)c3ccc(-c4sc(-c5ccccc5)c5c4OCCO5)cc3)cc21 ; CC1(C)c2ccccc2-c2c(N(c3ccc(-c4ccccc4)cc3)c3ccc(-c4sc(-c5ccccc5)c5c4OCCO5)cc3)cccc21]

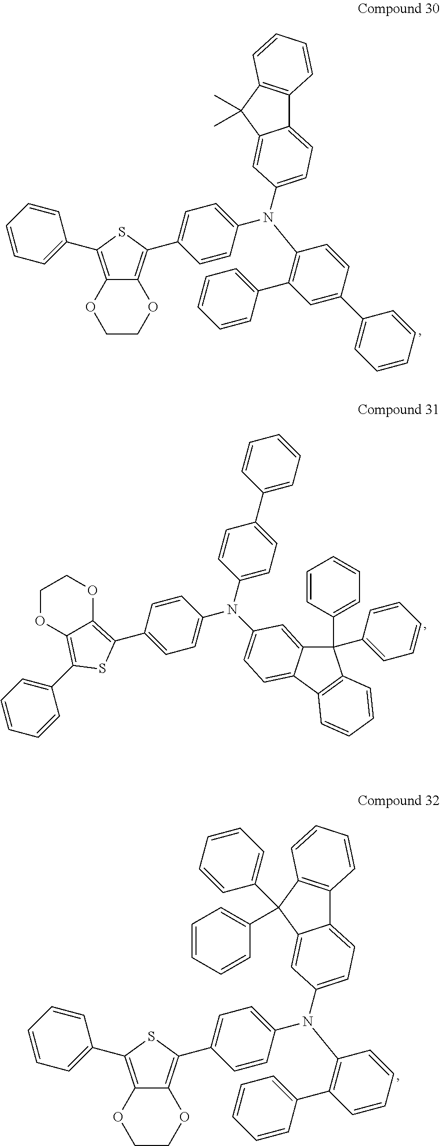 [SMILES: CC1(C)c2ccccc2-c2ccc(N(c3ccc(-c4sc(-c5ccccc5)c5c4OCCO5)cc3)c3ccc(-c4ccccc4)cc3-c3ccccc3)cc21 ; c1ccc(-c2ccc(N(c3ccc(-c4sc(-c5ccccc5)c5c4OCCO5)cc3)c3ccc4c(c3)C(c3ccccc3)(c3ccccc3)c3ccccc3-4)cc2)cc1 ; c1ccc(-c2ccccc2N(c2ccc(-c3sc(-c4ccccc4)c4c3OCCO4)cc2)c2ccc3c(c2)C(c2ccccc2)(c2ccccc2)c2ccccc2-3)cc1]

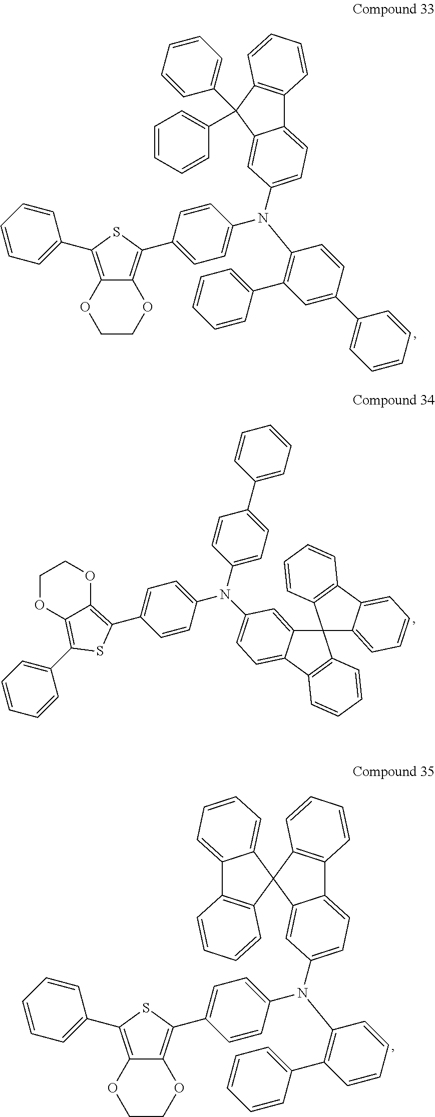 [SMILES: c1ccc(-c2ccc(N(c3ccc(-c4sc(-c5ccccc5)c5c4OCCO5)cc3)c3ccc4c(c3)C(c3ccccc3)(c3ccccc3)c3ccccc3-4)c(-c3ccccc3)c2)cc1 ; c1ccc(-c2ccc(N(c3ccc(-c4sc(-c5ccccc5)c5c4OCCO5)cc3)c3ccc4c(c3)C3(c5ccccc5-c5ccccc53)c3ccccc3-4)cc2)cc1 ; c1ccc(-c2ccccc2N(c2ccc(-c3sc(-c4ccccc4)c4c3OCCO4)cc2)c2ccc3c(c2)C2(c4ccccc4-c4ccccc42)c2ccccc2-3)cc1]

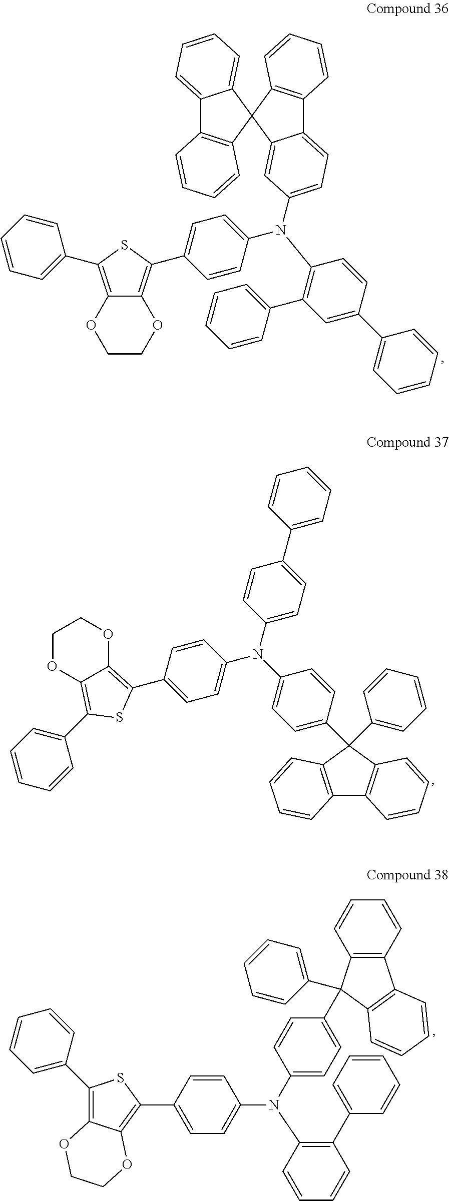 [SMILES: c1ccc(-c2ccc(N(c3ccc(-c4sc(-c5ccccc5)c5c4OCCO5)cc3)c3ccc4c(c3)C3(c5ccccc5-c5ccccc53)c3ccccc3-4)c(-c3ccccc3)c2)cc1 ; c1ccc(-c2ccc(N(c3ccc(-c4sc(-c5ccccc5)c5c4OCCO5)cc3)c3ccc(C4(c5ccccc5)c5ccccc5-c5ccccc54)cc3)cc2)cc1 ; c1ccc(-c2ccccc2N(c2ccc(-c3sc(-c4ccccc4)c4c3OCCO4)cc2)c2ccc(C3(c4ccccc4)c4ccccc4-c4ccccc43)cc2)cc1]

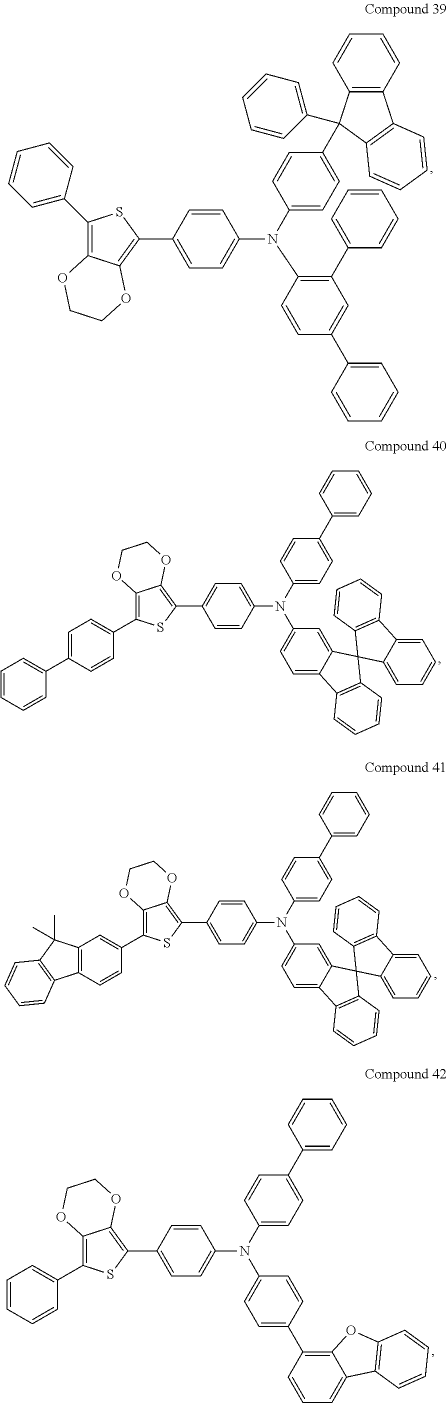 [SMILES: c1ccc(-c2ccc(N(c3ccc(-c4sc(-c5ccccc5)c5c4OCCO5)cc3)c3ccc(C4(c5ccccc5)c5ccccc5-c5ccccc54)cc3)c(-c3ccccc3)c2)cc1 ; c1ccc(-c2ccc(-c3sc(-c4ccc(N(c5ccc(-c6ccccc6)cc5)c5ccc6c(c5)C5(c7ccccc7-c7ccccc75)c5ccccc5-6)cc4)c4c3OCCO4)cc2)cc1 ; CC1(C)c2ccccc2-c2ccc(-c3sc(-c4ccc(N(c5ccc(-c6ccccc6)cc5)c5ccc6c(c5)C5(c7ccccc7-c7ccccc75)c5ccccc5-6)cc4)c4c3OCCO4)cc21 ; c1ccc(-c2ccc(N(c3ccc(-c4sc(-c5ccccc5)c5c4OCCO5)cc3)c3ccc(-c4cccc5c4oc4ccccc45)cc3)cc2)cc1]

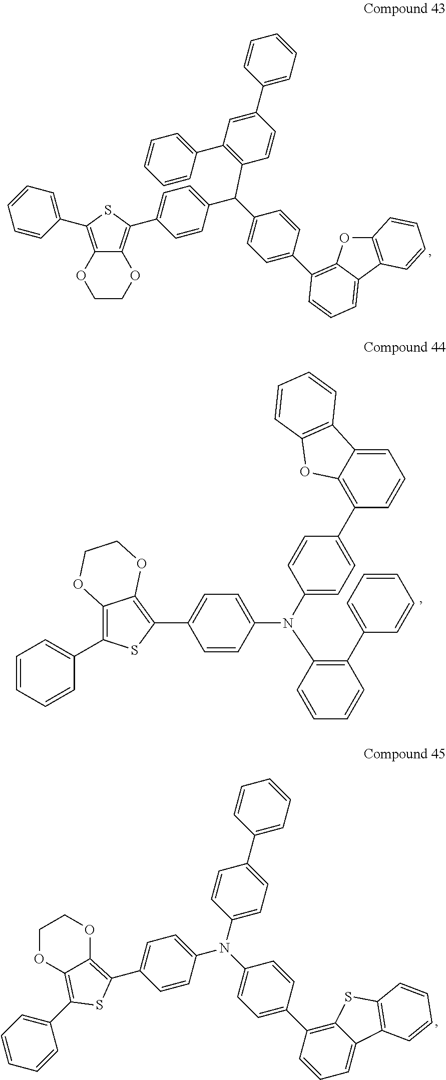 [SMILES: c1ccc(-c2ccc(C(c3ccc(-c4sc(-c5ccccc5)c5c4OCCO5)cc3)c3ccc(-c4cccc5c4oc4ccccc45)cc3)c(-c3ccccc3)c2)cc1 ; c1ccc(-c2ccccc2N(c2ccc(-c3sc(-c4ccccc4)c4c3OCCO4)cc2)c2ccc(-c3cccc4c3oc3ccccc34)cc2)cc1 ; c1ccc(-c2ccc(N(c3ccc(-c4sc(-c5ccccc5)c5c4OCCO5)cc3)c3ccc(-c4cccc5c4sc4ccccc45)cc3)cc2)cc1]

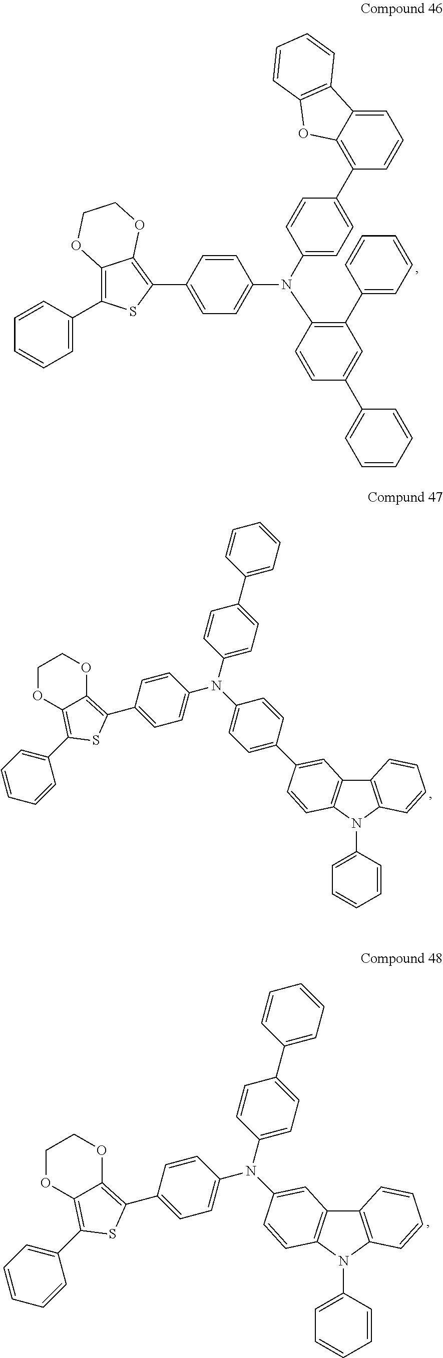 [SMILES: c1ccc(-c2ccc(N(c3ccc(-c4sc(-c5ccccc5)c5c4OCCO5)cc3)c3ccc(-c4cccc5c4oc4ccccc45)cc3)c(-c3ccccc3)c2)cc1 ; c1ccc(-c2ccc(N(c3ccc(-c4ccc5c(c4)c4ccccc4n5-c4ccccc4)cc3)c3ccc(-c4sc(-c5ccccc5)c5c4OCCO5)cc3)cc2)cc1 ; c1ccc(-c2ccc(N(c3ccc(-c4sc(-c5ccccc5)c5c4OCCO5)cc3)c3ccc4c(c3)c3ccccc3n4-c3ccccc3)cc2)cc1]

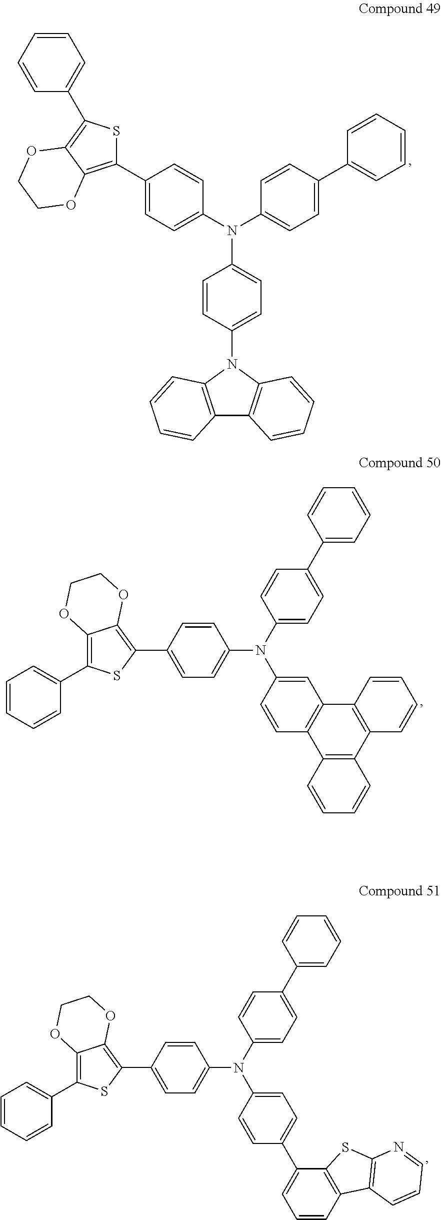 [SMILES: c1ccc(-c2ccc(N(c3ccc(-c4sc(-c5ccccc5)c5c4OCCO5)cc3)c3ccc(-n4c5ccccc5c5ccccc54)cc3)cc2)cc1 ; c1ccc(-c2ccc(N(c3ccc(-c4sc(-c5ccccc5)c5c4OCCO5)cc3)c3ccc4c5ccccc5c5ccccc5c4c3)cc2)cc1 ; c1ccc(-c2ccc(N(c3ccc(-c4sc(-c5ccccc5)c5c4OCCO5)cc3)c3ccc(-c4cccc5c4sc4ncccc45)cc3)cc2)cc1]

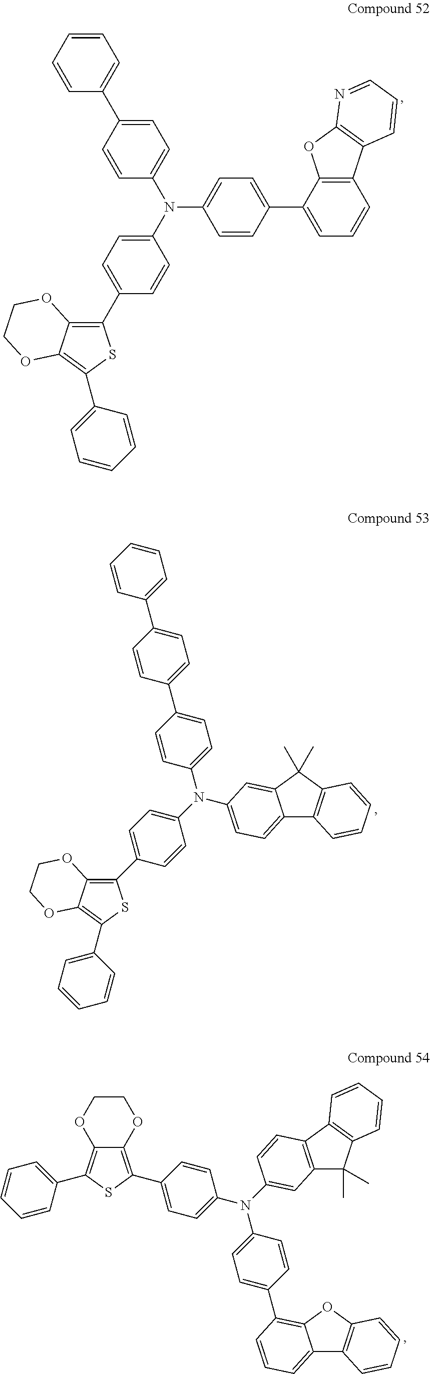 [SMILES: c1ccc(-c2ccc(N(c3ccc(-c4sc(-c5ccccc5)c5c4OCCO5)cc3)c3ccc(-c4cccc5c4oc4ncccc45)cc3)cc2)cc1 ; CC1(C)c2ccccc2-c2ccc(N(c3ccc(-c4ccc(-c5ccccc5)cc4)cc3)c3ccc(-c4sc(-c5ccccc5)c5c4OCCO5)cc3)cc21 ; CC1(C)c2ccccc2-c2ccc(N(c3ccc(-c4sc(-c5ccccc5)c5c4OCCO5)cc3)c3ccc(-c4cccc5c4oc4ccccc45)cc3)cc21]

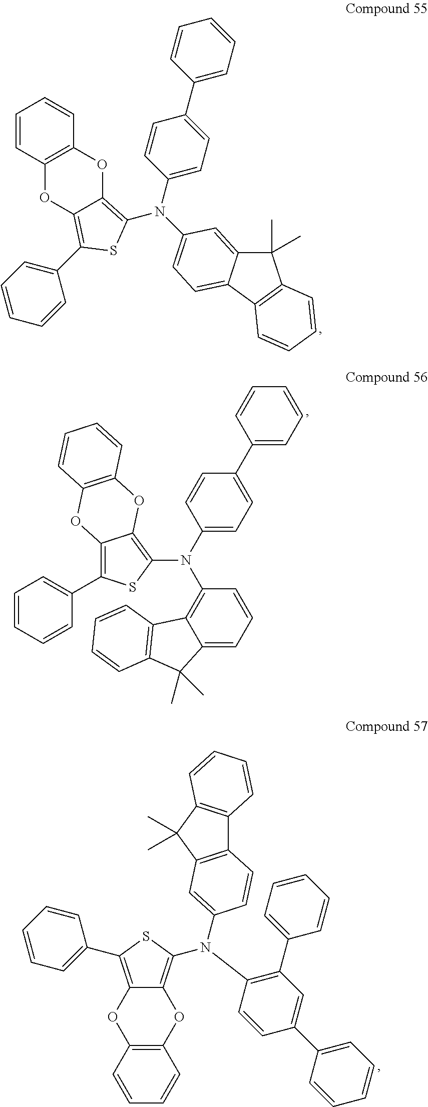

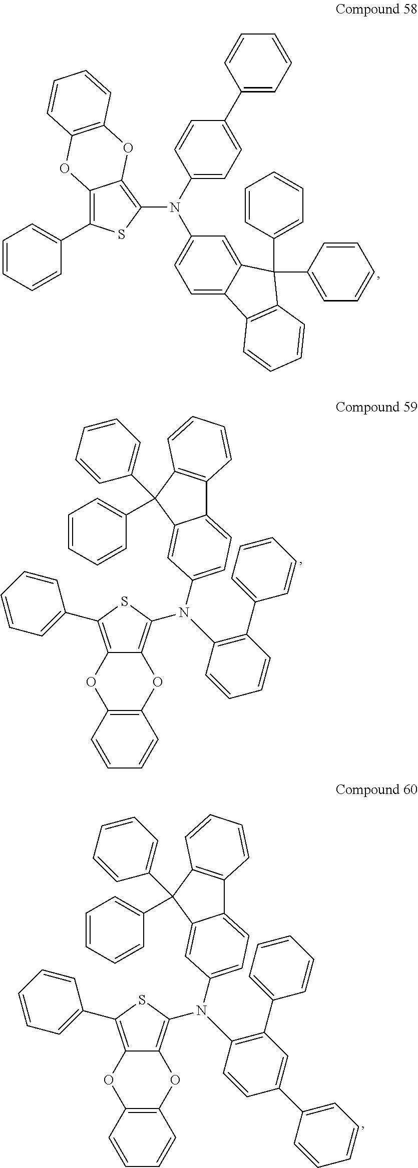 [SMILES: c1ccc(-c2ccc(N(c3ccc4c(c3)C(c3ccccc3)(c3ccccc3)c3ccccc3-4)c3sc(-c4ccccc4)c4c3Oc3ccccc3O4)cc2)cc1 ; c1ccc(-c2ccccc2N(c2ccc3c(c2)C(c2ccccc2)(c2ccccc2)c2ccccc2-3)c2sc(-c3ccccc3)c3c2Oc2ccccc2O3)cc1 ; c1ccc(-c2ccc(N(c3ccc4c(c3)C(c3ccccc3)(c3ccccc3)c3ccccc3-4)c3sc(-c4ccccc4)c4c3Oc3ccccc3O4)c(-c3ccccc3)c2)cc1]

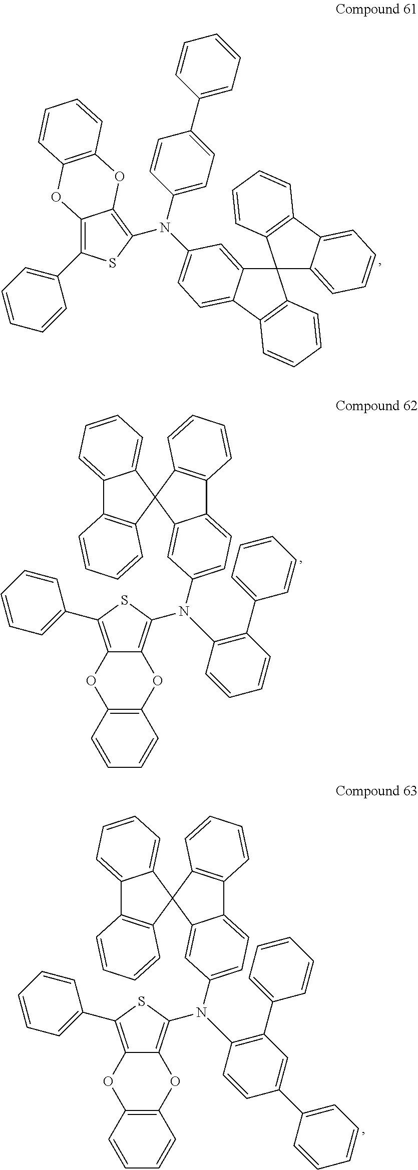 [SMILES: c1ccc(-c2ccc(N(c3ccc4c(c3)C3(c5ccccc5-c5ccccc53)c3ccccc3-4)c3sc(-c4ccccc4)c4c3Oc3ccccc3O4)cc2)cc1 ; c1ccc(-c2ccccc2N(c2ccc3c(c2)C2(c4ccccc4-c4ccccc42)c2ccccc2-3)c2sc(-c3ccccc3)c3c2Oc2ccccc2O3)cc1 ; c1ccc(-c2ccc(N(c3ccc4c(c3)C3(c5ccccc5-c5ccccc53)c3ccccc3-4)c3sc(-c4ccccc4)c4c3Oc3ccccc3O4)c(-c3ccccc3)c2)cc1]

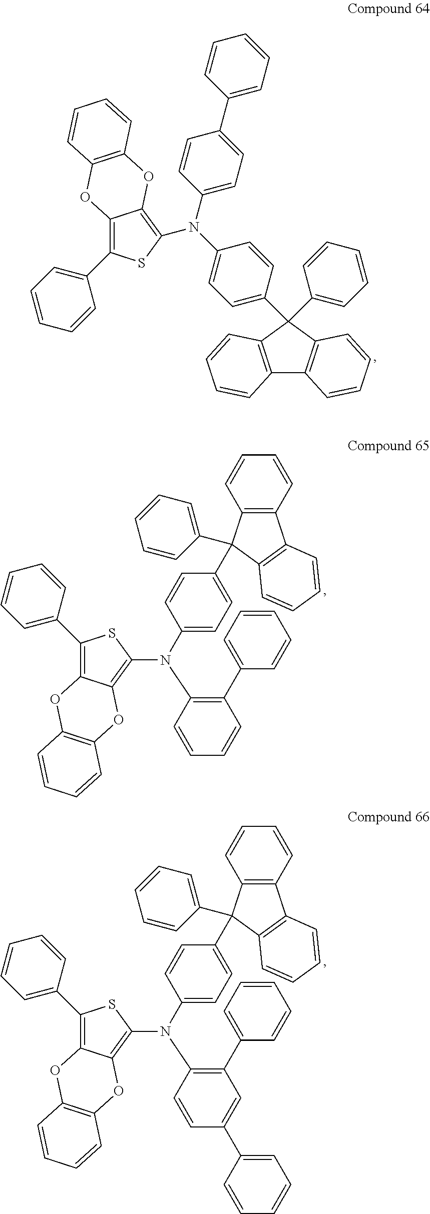 [SMILES: c1ccc(-c2ccc(N(c3ccc(C4(c5ccccc5)c5ccccc5-c5ccccc54)cc3)c3sc(-c4ccccc4)c4c3Oc3ccccc3O4)cc2)cc1 ; c1ccc(-c2ccccc2N(c2ccc(C3(c4ccccc4)c4ccccc4-c4ccccc43)cc2)c2sc(-c3ccccc3)c3c2Oc2ccccc2O3)cc1 ; c1ccc(-c2ccc(N(c3ccc(C4(c5ccccc5)c5ccccc5-c5ccccc54)cc3)c3sc(-c4ccccc4)c4c3Oc3ccccc3O4)c(-c3ccccc3)c2)cc1]

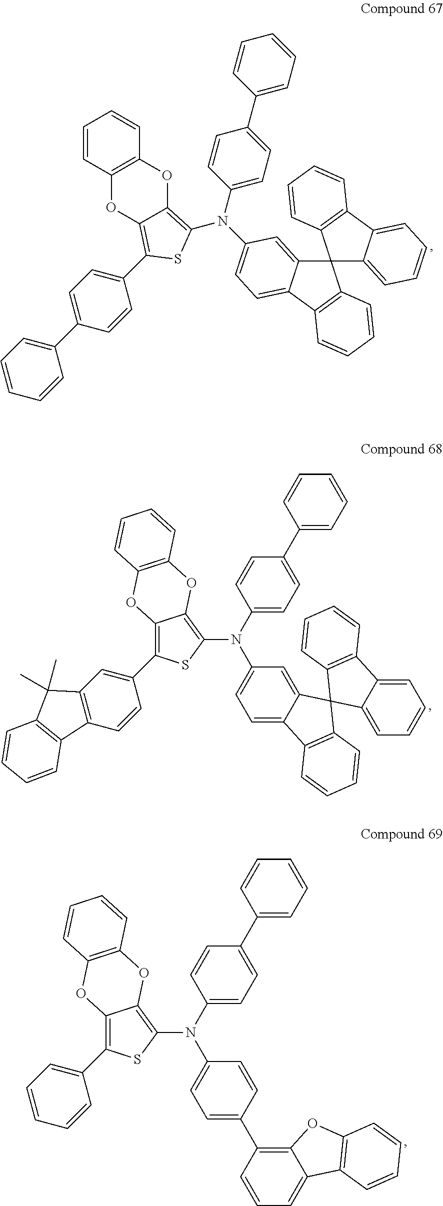 [SMILES: c1ccc(-c2ccc(-c3sc(N(c4ccc(-c5ccccc5)cc4)c4ccc5c(c4)C4(c6ccccc6-c6ccccc64)c4ccccc4-5)c4c3Oc3ccccc3O4)cc2)cc1 ; CC1(C)c2ccccc2-c2ccc(-c3sc(N(c4ccc(-c5ccccc5)cc4)c4ccc5c(c4)C4(c6ccccc6-c6ccccc64)c4ccccc4-5)c4c3Oc3ccccc3O4)cc21 ; c1ccc(-c2ccc(N(c3ccc(-c4cccc5c4oc4ccccc45)cc3)c3sc(-c4ccccc4)c4c3Oc3ccccc3O4)cc2)cc1]

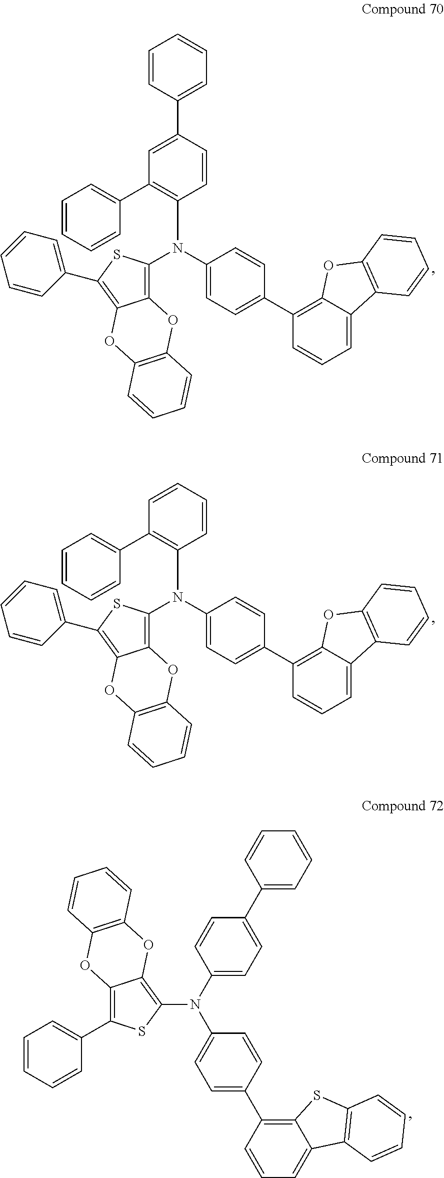 [SMILES: c1ccc(-c2ccc(N(c3ccc(-c4cccc5c4oc4ccccc45)cc3)c3sc(-c4ccccc4)c4c3Oc3ccccc3O4)c(-c3ccccc3)c2)cc1 ; c1ccc(-c2ccccc2N(c2ccc(-c3cccc4c3oc3ccccc34)cc2)c2sc(-c3ccccc3)c3c2Oc2ccccc2O3)cc1 ; c1ccc(-c2ccc(N(c3ccc(-c4cccc5c4sc4ccccc45)cc3)c3sc(-c4ccccc4)c4c3Oc3ccccc3O4)cc2)cc1]

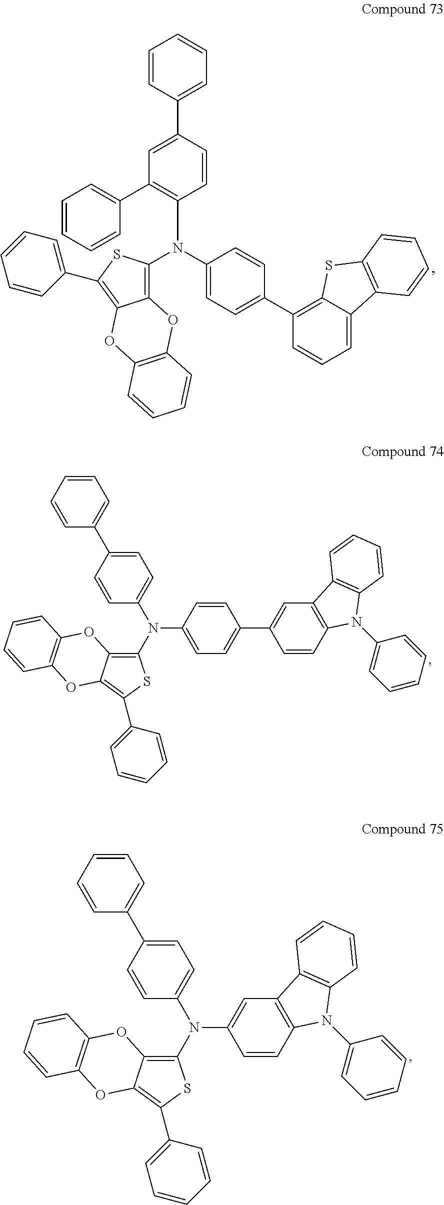 [SMILES: c1ccc(-c2ccc(N(c3ccc(-c4cccc5c4sc4ccccc45)cc3)c3sc(-c4ccccc4)c4c3Oc3ccccc3O4)c(-c3ccccc3)c2)cc1 ; c1ccc(-c2ccc(N(c3ccc(-c4ccc5c(c4)c4ccccc4n5-c4ccccc4)cc3)c3sc(-c4ccccc4)c4c3Oc3ccccc3O4)cc2)cc1 ; c1ccc(-c2ccc(N(c3ccc4c(c3)c3ccccc3n4-c3ccccc3)c3sc(-c4ccccc4)c4c3Oc3ccccc3O4)cc2)cc1]

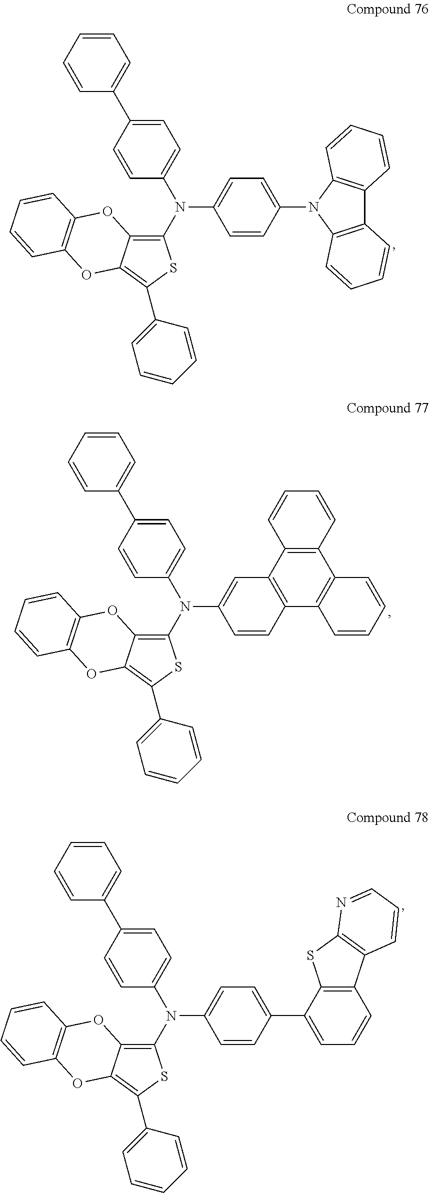 [SMILES: c1ccc(-c2ccc(N(c3ccc(-n4c5ccccc5c5ccccc54)cc3)c3sc(-c4ccccc4)c4c3Oc3ccccc3O4)cc2)cc1 ; c1ccc(-c2ccc(N(c3ccc4c5ccccc5c5ccccc5c4c3)c3sc(-c4ccccc4)c4c3Oc3ccccc3O4)cc2)cc1 ; c1ccc(-c2ccc(N(c3ccc(-c4cccc5c4sc4ncccc45)cc3)c3sc(-c4ccccc4)c4c3Oc3ccccc3O4)cc2)cc1]

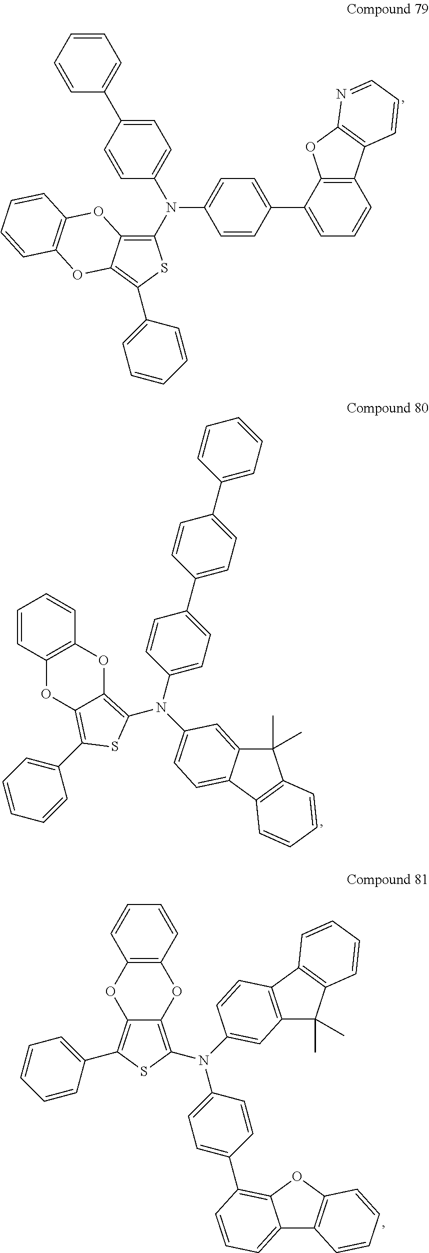 [SMILES: c1ccc(-c2ccc(N(c3ccc(-c4cccc5c4oc4ncccc45)cc3)c3sc(-c4ccccc4)c4c3Oc3ccccc3O4)cc2)cc1 ; CC1(C)c2ccccc2-c2ccc(N(c3ccc(-c4ccc(-c5ccccc5)cc4)cc3)c3sc(-c4ccccc4)c4c3Oc3ccccc3O4)cc21 ; CC1(C)c2ccccc2-c2ccc(N(c3ccc(-c4cccc5c4oc4ccccc45)cc3)c3sc(-c4ccccc4)c4c3Oc3ccccc3O4)cc21]

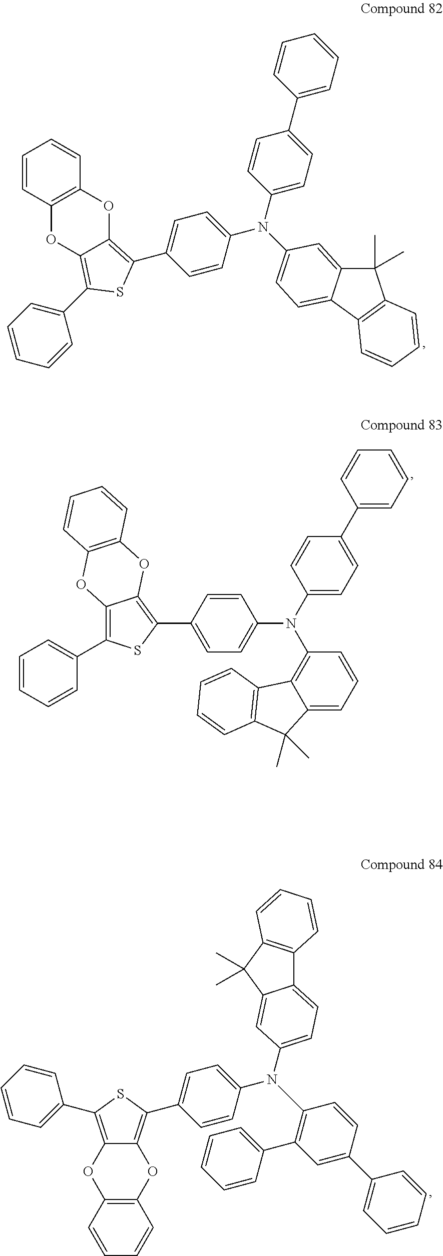 [SMILES: CC1(C)c2ccccc2-c2ccc(N(c3ccc(-c4ccccc4)cc3)c3ccc(-c4sc(-c5ccccc5)c5c4Oc4ccccc4O5)cc3)cc21 ; CC1(C)c2ccccc2-c2c(N(c3ccc(-c4ccccc4)cc3)c3ccc(-c4sc(-c5ccccc5)c5c4Oc4ccccc4O5)cc3)cccc21 ; CC1(C)c2ccccc2-c2ccc(N(c3ccc(-c4sc(-c5ccccc5)c5c4Oc4ccccc4O5)cc3)c3ccc(-c4ccccc4)cc3-c3ccccc3)cc21]

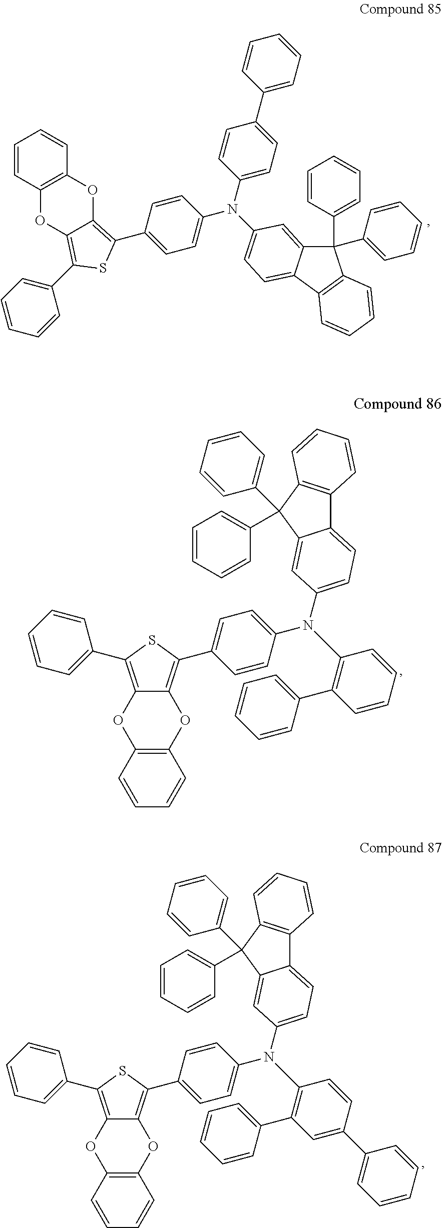 [SMILES: c1ccc(-c2ccc(N(c3ccc(-c4sc(-c5ccccc5)c5c4Oc4ccccc4O5)cc3)c3ccc4c(c3)C(c3ccccc3)(c3ccccc3)c3ccccc3-4)cc2)cc1 ; c1ccc(-c2ccccc2N(c2ccc(-c3sc(-c4ccccc4)c4c3Oc3ccccc3O4)cc2)c2ccc3c(c2)C(c2ccccc2)(c2ccccc2)c2ccccc2-3)cc1 ; c1ccc(-c2ccc(N(c3ccc(-c4sc(-c5ccccc5)c5c4Oc4ccccc4O5)cc3)c3ccc4c(c3)C(c3ccccc3)(c3ccccc3)c3ccccc3-4)c(-c3ccccc3)c2)cc1]

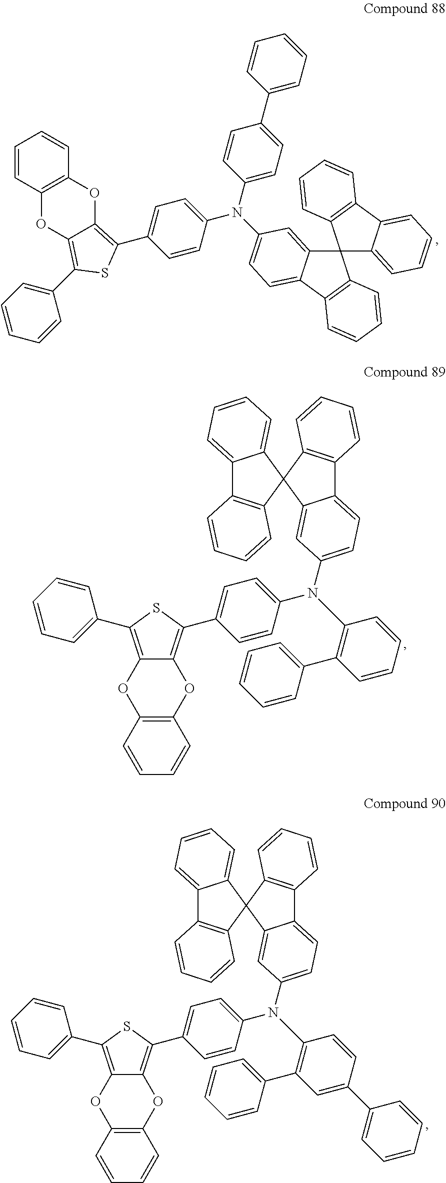 [SMILES: c1ccc(-c2ccc(N(c3ccc(-c4sc(-c5ccccc5)c5c4Oc4ccccc4O5)cc3)c3ccc4c(c3)C3(c5ccccc5-c5ccccc53)c3ccccc3-4)cc2)cc1 ; c1ccc(-c2ccccc2N(c2ccc(-c3sc(-c4ccccc4)c4c3Oc3ccccc3O4)cc2)c2ccc3c(c2)C2(c4ccccc4-c4ccccc42)c2ccccc2-3)cc1 ; c1ccc(-c2ccc(N(c3ccc(-c4sc(-c5ccccc5)c5c4Oc4ccccc4O5)cc3)c3ccc4c(c3)C3(c5ccccc5-c5ccccc53)c3ccccc3-4)c(-c3ccccc3)c2)cc1]

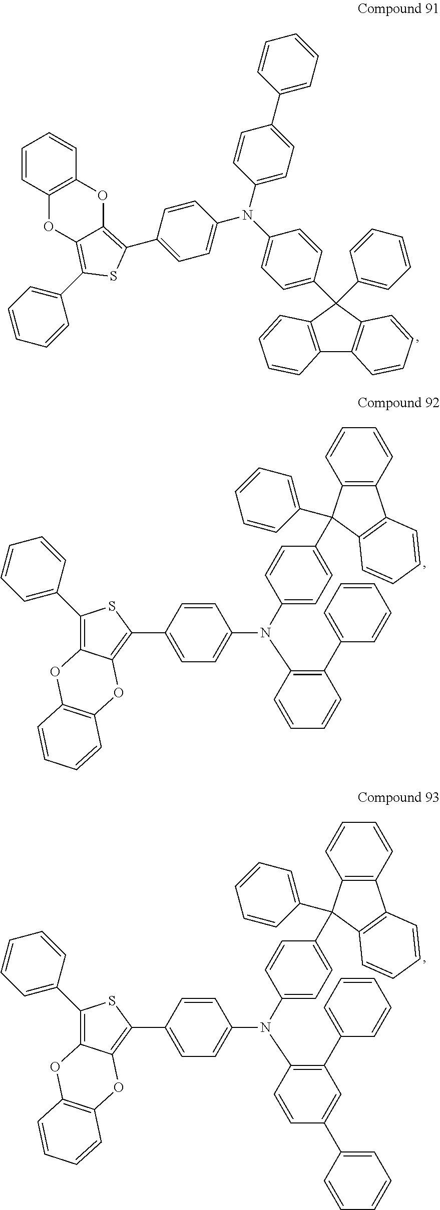 [SMILES: c1ccc(-c2ccc(N(c3ccc(-c4sc(-c5ccccc5)c5c4Oc4ccccc4O5)cc3)c3ccc(C4(c5ccccc5)c5ccccc5-c5ccccc54)cc3)cc2)cc1 ; c1ccc(-c2ccccc2N(c2ccc(-c3sc(-c4ccccc4)c4c3Oc3ccccc3O4)cc2)c2ccc(C3(c4ccccc4)c4ccccc4-c4ccccc43)cc2)cc1 ; c1ccc(-c2ccc(N(c3ccc(-c4sc(-c5ccccc5)c5c4Oc4ccccc4O5)cc3)c3ccc(C4(c5ccccc5)c5ccccc5-c5ccccc54)cc3)c(-c3ccccc3)c2)cc1]

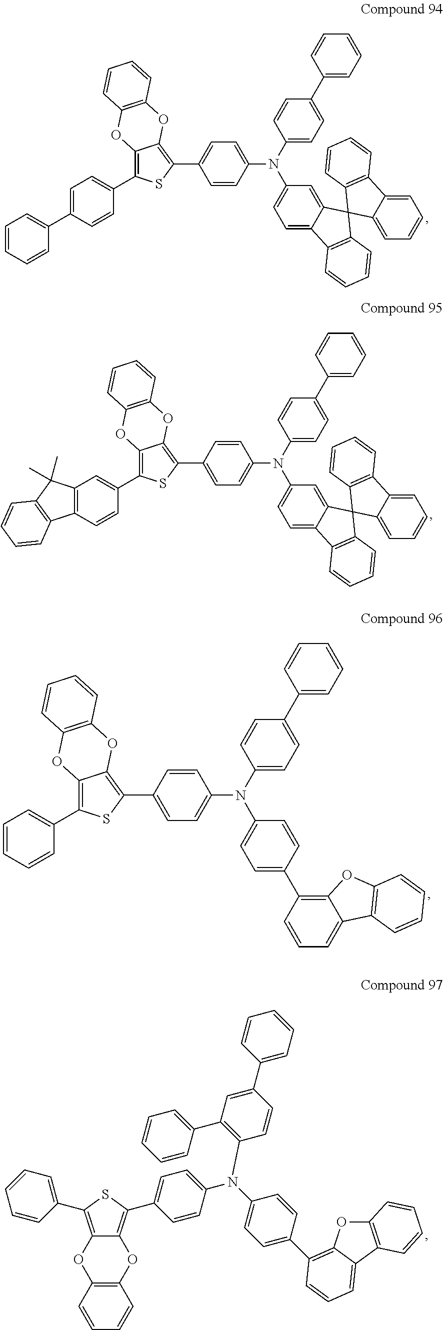 [SMILES: c1ccc(-c2ccc(-c3sc(-c4ccc(N(c5ccc(-c6ccccc6)cc5)c5ccc6c(c5)C5(c7ccccc7-c7ccccc75)c5ccccc5-6)cc4)c4c3Oc3ccccc3O4)cc2)cc1 ; CC1(C)c2ccccc2-c2ccc(-c3sc(-c4ccc(N(c5ccc(-c6ccccc6)cc5)c5ccc6c(c5)C5(c7ccccc7-c7ccccc75)c5ccccc5-6)cc4)c4c3Oc3ccccc3O4)cc21 ; c1ccc(-c2ccc(N(c3ccc(-c4sc(-c5ccccc5)c5c4Oc4ccccc4O5)cc3)c3ccc(-c4cccc5c4oc4ccccc45)cc3)cc2)cc1 ; c1ccc(-c2ccc(N(c3ccc(-c4sc(-c5ccccc5)c5c4Oc4ccccc4O5)cc3)c3ccc(-c4cccc5c4oc4ccccc45)cc3)c(-c3ccccc3)c2)cc1]

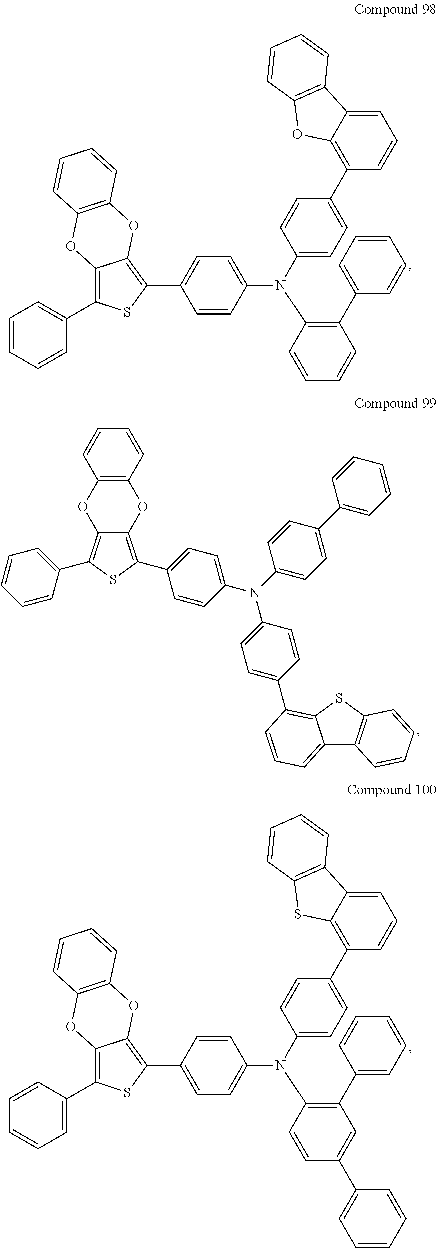 [SMILES: c1ccc(-c2ccccc2N(c2ccc(-c3sc(-c4ccccc4)c4c3Oc3ccccc3O4)cc2)c2ccc(-c3cccc4c3oc3ccccc34)cc2)cc1 ; c1ccc(-c2ccc(N(c3ccc(-c4sc(-c5ccccc5)c5c4Oc4ccccc4O5)cc3)c3ccc(-c4cccc5c4sc4ccccc45)cc3)cc2)cc1 ; c1ccc(-c2ccc(N(c3ccc(-c4sc(-c5ccccc5)c5c4Oc4ccccc4O5)cc3)c3ccc(-c4cccc5c4sc4ccccc45)cc3)c(-c3ccccc3)c2)cc1]

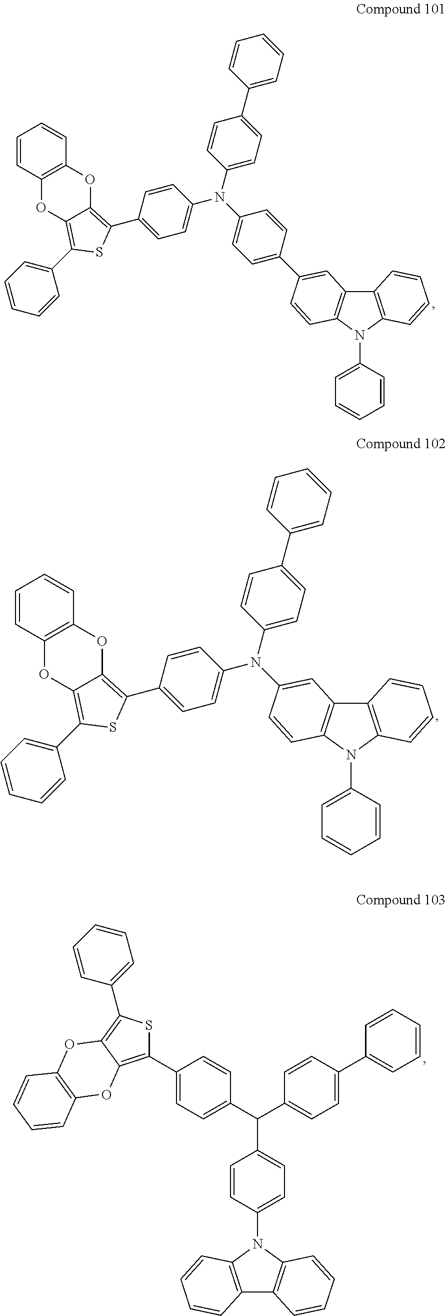 [SMILES: c1ccc(-c2ccc(N(c3ccc(-c4ccc5c(c4)c4ccccc4n5-c4ccccc4)cc3)c3ccc(-c4sc(-c5ccccc5)c5c4Oc4ccccc4O5)cc3)cc2)cc1 ; c1ccc(-c2ccc(N(c3ccc(-c4sc(-c5ccccc5)c5c4Oc4ccccc4O5)cc3)c3ccc4c(c3)c3ccccc3n4-c3ccccc3)cc2)cc1 ; c1ccc(-c2ccc(C(c3ccc(-c4sc(-c5ccccc5)c5c4Oc4ccccc4O5)cc3)c3ccc(-n4c5ccccc5c5ccccc54)cc3)cc2)cc1]

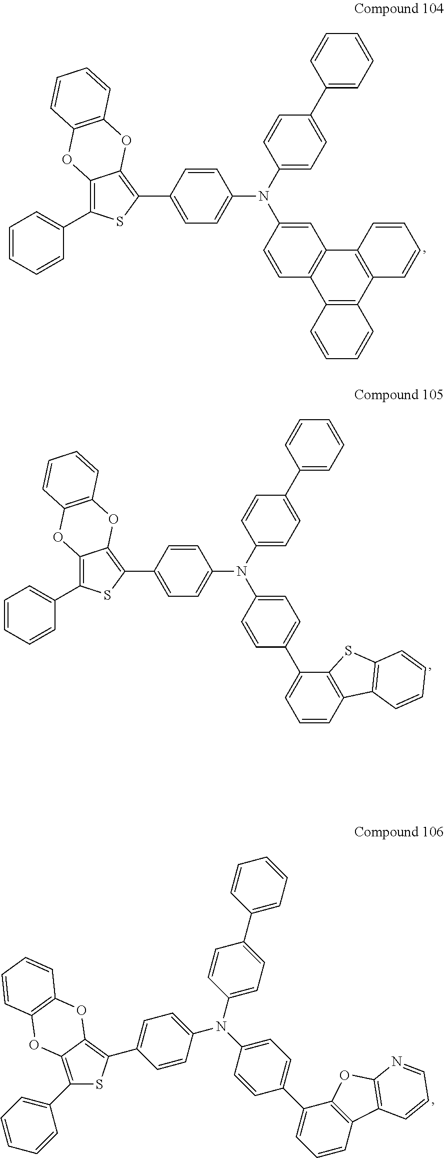 [SMILES: c1ccc(-c2ccc(N(c3ccc(-c4sc(-c5ccccc5)c5c4Oc4ccccc4O5)cc3)c3ccc4c5ccccc5c5ccccc5c4c3)cc2)cc1 ; c1ccc(-c2ccc(N(c3ccc(-c4sc(-c5ccccc5)c5c4Oc4ccccc4O5)cc3)c3ccc(-c4cccc5c4sc4ccccc45)cc3)cc2)cc1 ; c1ccc(-c2ccc(N(c3ccc(-c4sc(-c5ccccc5)c5c4Oc4ccccc4O5)cc3)c3ccc(-c4cccc5c4oc4ncccc45)cc3)cc2)cc1]

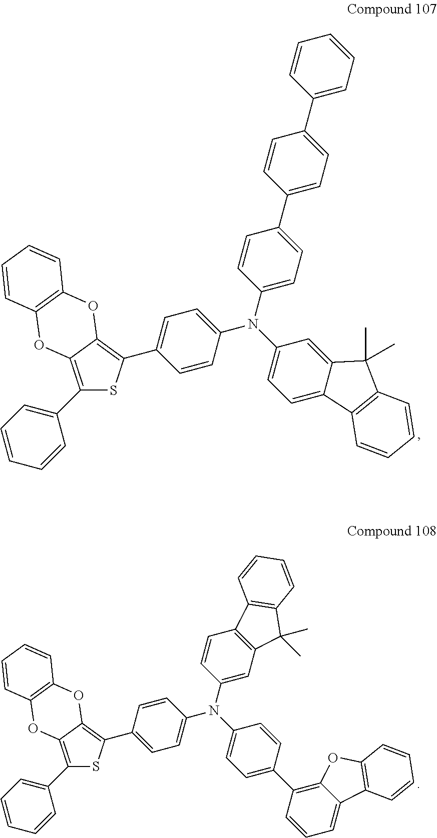 [SMILES: CC1(C)c2ccccc2-c2ccc(N(c3ccc(-c4ccc(-c5ccccc5)cc4)cc3)c3ccc(-c4sc(-c5ccccc5)c5c4Oc4ccccc4O5)cc3)cc21 ; CC1(C)c2ccccc2-c2ccc(N(c3ccc(-c4sc(-c5ccccc5)c5c4Oc4ccccc4O5)cc3)c3ccc(-c4cccc5c4oc4ccccc45)cc3)cc21]

8. An organic electroluminescent device comprising:
an anode,
a cathode,
a series of organic layers disposed between the anode and cathode, wherein at least one of the organic layers comprises a compound having Formula 1: 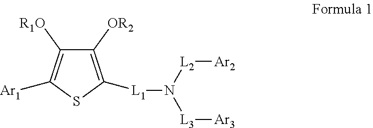
wherein
R₁ and R₂ are each independently selected from the group consisting of a substituted or unsubstituted alkyl group having 1 to 20 carbon atoms, a substituted or unsubstituted cycloalkyl group having 3 to 20 ring carbon atoms, a substituted or unsubstituted heteroalkyl group having 1 to 20 carbon atoms, a substituted or unsubstituted arylalkyl group having 7 to 30 carbon atoms, a substituted or unsubstituted alkoxy group having 1 to 20 carbon atoms, a substituted or unsubstituted aryloxy group having 6 to 30 carbon atoms, a substituted or unsubstituted alkenyl group having 2 to 20 carbon atoms, a substituted or unsubstituted aryl group having 6 to 30 carbon atoms, a substituted or unsubstituted heteroaryl group having 3 to 30 carbon atoms, and combinations thereof;
R₁ and R₂ are optionally joined to form a ring;
each of Ar₁, Ar₂, and Ar₃ are independently selected from the group consisting of a substituted or unsubstituted aryl group having 6 to 50 ring carbon atoms, a substituted or unsubstituted heteroaryl group having 5 to 50 ring atoms, and combinations thereof;
each of L₁, L₂, and L₃ are independently selected from the group consisting of a single bond, an arylene group having 6 to 30 ring carbon atoms, a heteroarylene group having 5 to 30 ring atoms, and combinations thereof.

9. The organic electroluminescent device of claim 8, wherein the device comprises a hole transporting layer, wherein the hole transporting layer comprises the compound of Formula 1.

10. The organic electroluminescent device of claim 8, wherein the device comprises a hole injection layer, wherein the hole injection layer comprises the compound of Formula 1.

11. The organic electroluminescent device of claim 9, wherein the hole transporting layer further comprises a p-type conductivity dopant.

12. The organic electroluminescent device of claim 10, wherein the hole injection layer further comprises a p-type conductivity dopant.

13. A formulation comprising the compound of claim 1.
